(19) United States
(12) Patent Application Publication
(10) Pub. No.: US 2022/0098616 A1
(43) Pub. Date: Mar. 31, 2022

(54) ISL1 and LHX3 VECTOR

(71) Applicant: NeuExcell Therapeutics Inc., State College, PA (US)

(72) Inventor: Jie XU, State College, PA (US)

(73) Assignee: NeuExcell Therapeutics Inc., State College, PA (US)

(21) Appl. No.: 17/487,342

(22) Filed: Sep. 28, 2021

Related U.S. Application Data
(60) Provisional application No. 63/084,962, filed on Sep. 29, 2020.
(60) Provisional application No. 63/247,561, filed on Sep. 23, 2021.

(51) Int. Cl. C12N 15/86; C12N 5/079; C12N 5/0793; C07K 14/47; A61P 25/00; A61P 25/14

(52) U.S. Cl. CPC C12N 15/86; C12N 5/0622; C12N 5/0619; C07K 14/4705; C07K 14/47; A61K 38/00; A61P 25/14; C12N 2710/10043; C12N 2710/16143; C12N 2506/08; A61P 25/00

(57) ABSTRACT

The present disclosure relates to AAV vectors, compositions, and methods related to converting glial cells to neurons by the use of ISL1 and LHX3 coding sequences in an AAV vector.

### CROSS-REFERENCE TO RELATED APPLICATION

This application is a U.S. patent application which claims the benefit and priority to U.S. Provisional Application Nos. 63/084,962 filed Sep. 29, 2020, and 63/247,561 filed Sep. 23, 2021, each of which are incorporated by reference in their entireties herein.

### INCORPORATION BY REFERENCE

A sequence listing contained in the file named P34839US02_SL.txt which is 25,752 bytes (measured in MS-Windows®) and created on Sep. 27, 2021, is filed electronically herewith and incorporated by reference in its entirety.

### FIELD OF THE INVENTION

The present disclosure includes methods and compositions using an AAV vector comprising a nucleic acid sequence encoding human ISL1 and LHX3 to convert glial cells to neurons.

### BACKGROUND OF THE INVENTION

Neurons are often killed or damaged and unable to regenerate in subjects with a neurological condition or following an injury to the central nervous system (CNS) or peripheral nervous system (PNS).

Glial cells become reactive following an injury to the CNS or PNS such as a brain injury or neurological condition.

Currently there are no methods available to regenerate functional new neurons in human subjects having a neurological condition using adeno-associated viruses (AAVs).

### SUMMARY OF THE INVENTION

In one aspect, this disclosure provides, and includes, an adeno-associated virus (AAV) vector comprising a human insulin gene enhancer protein (hISL1) sequence comprising the nucleic acid sequence of SEQ ID NO: 6 and a human LIM-homeobox 3 (hLHX3) sequence comprising the nucleic acid sequence of SEQ ID NO: 13, where the hISL1 sequence and the hLHX3 sequence are separated by a P2A linker comprising the nucleic acid sequence selected from the group consisting of SEQ ID NO: 15 and 18 or a T2A linker comprising the nucleic acid sequence selected from the group consisting of SEQ ID NO: 16 and 19, where the hISL1 sequence and the hLHX3 sequence are operably linked to regulatory elements comprising: (a) glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In one aspect, this disclosure provides, and includes, an adeno-associated virus (AAV) vector comprising a nucleic acid coding sequence encoding a human insulin gene enhancer protein (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, where the hISL1 coding sequence and the hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 coding sequence and the hLHX3 coding sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising an insulin gene enhancer protein (ISL1) nucleic acid coding sequence encoding a ISL1 protein and a LIM-homeobox 3 (LHX3) nucleic acid coding sequence encoding a LHX3 protein, where the ISL1 coding sequence and the LHX3 coding sequence are separated by a linker sequence, where the ISL1 coding sequence and the LHX3 coding sequence are operably linked to regulatory elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human insulin gene enhancer protein (hISL1) sequence having a nucleic acid sequence of SEQ ID NO: 6 and a human LIM-homeobox 3 (hLHX3) sequence having a nucleic acid sequence of SEQ ID NO: 13, where the hISL1 sequence and the hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 sequence and hLHX3 sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated-virus (AAV) vector for converting glial cells to functional neurons in a human, where the AAV vector comprises a nucleic acid coding sequence encoding a human insulin gene enhancer protein (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, where the hISL1 coding sequence and the hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 coding sequence and the hLHX3 coding sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated virus (AAV) vector for the treatment of a subject in need thereof, where the AAV vector comprises an insulin gene enhancer protein (ISL1) sequence and a LIM-homeobox 3 (LHX3) sequence, where the ISL1 sequence and the LHX3 sequence are separated by a linker sequence, where the ISL1 sequence and LHX3 sequence are operably linked to expression control elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a human insulin gene enhancer protein (hISL1) sequence comprising the nucleic acid sequence of SEQ ID NO: 6 and a human LIM-homeobox 3 (hLHX3) sequence comprising the nucleic acid sequence of SEQ ID NO: 13, where the hISL1 sequence and the hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 sequence and the hLHX3 sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a nucleic acid coding sequence encoding a human insulin gene enhancer protein (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, where the hISL1 coding sequence and the hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 coding sequence and hLHX3 coding sequence are operably linked to expression control elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting glial cells to neurons in a subject in need thereof comprising: delivering an adeno-associated virus (AAV) to the subject in need thereof, where the AAV comprises a DNA vector construct comprising an insulin gene enhancer protein (ISL1) sequence and a LIM-homeobox 3 (LHX3) sequence, where the ISL1 sequence and LHX3 sequence are separated by a linker sequence, where the ISL1 sequence and LHX3 sequence are operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) and a polyadenylation signal sequence, where the vector is capable of converting at least one glial cell to a neuron in the subject in need thereof.

In an aspect, this disclosure provides, and includes, a method of treating a neurological condition in a subject in need thereof comprising: delivering an adeno-associated virus (AAV) to the subject, where the AAV comprises a DNA vector construct comprising an insulin gene enhancer protein (ISL1) sequence and LIM-homeobox 3 (LHX3) sequence, where the ISL1 sequence and LHX3 sequence are separated by a linker sequence, where the ISL1 sequence and the LHX3 sequence are operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal to the subject in need thereof.

In an aspect, this disclosure provides, and includes, an adeno-associated virus (AAV) vector comprising a human insulin gene enhancer protein (hISL1) sequence comprising the nucleic acid sequence of SEQ ID NO: 6, where the hISL1 sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising a nucleic acid coding sequence encoding a human insulin gene enhancer protein (hISL1) protein comprising the amino acid coding sequence of SEQ ID NO: 10, where the hISL1 coding sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising an insulin gene enhancer protein (ISL1) nucleic acid coding sequence encoding a ISL1 protein, where the ISL1 coding sequence is operably linked to regulatory elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human insulin gene enhancer protein (hISL1) sequence having a nucleic acid sequence of SEQ ID NO: 6, where the hISL1 sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated-virus (AAV) vector for converting glial cells to functional neurons in a human, where the AAV vector comprises a nucleic acid coding sequence encoding a human insulin gene enhancer protein (hISL1) protein comprising the amino acid sequence of SEQ ID NO: 10, where the hISL1 coding sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated virus (AAV) vector for the treatment of a subject in need thereof, where the AAV vector comprises an insulin gene enhancer protein (ISL1) sequence, where the ISL1 sequence is operably linked to expression control elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal.

In an aspect, this disclosure provides, and includes, an adeno-associated virus (AAV) vector comprising a human LIM-homeobox 3 (hLHX3) sequence comprising the nucleic acid sequence of SEQ ID NO: 13, where the hLHX3 sequence are operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising a nucleic acid coding sequence encoding a human LIM-homeobox 3 (hLHX3) protein comprising the amino acid sequence of SEQ ID NO: 14, where the hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 coding sequence and the hLHX3 coding sequence are operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising a LIM-homeobox 3 (LHX3) nucleic acid coding sequence encoding a LHX3 protein, where the LHX3 coding sequence is operably linked to regulatory elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human LIM-homeobox 3 (hLHX3) sequence having a nucleic acid sequence of SEQ ID NO: 13, where the hLHX3 sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated-virus (AAV) vector for converting glial cells to functional neurons in a human, where the AAV vector comprises a nucleic acid coding sequence encoding a human LIM-homeobox 3 (hLHX3) protein comprising the amino acid sequence of SEQ ID NO: 14, where the hLHX3 coding sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated virus (AAV) vector for the treatment of a subject in need thereof, where the AAV vector comprises a LIM-homeobox 3 (LHX3) sequence, where the LHX3 sequence are operably linked to expression control elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a human insulin gene enhancer protein (hISL1) sequence comprising the nucleic acid sequence of SEQ ID NO: 6, where the hISL1 sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a nucleic acid coding sequence encoding a human insulin gene enhancer protein (hISL1) protein comprising the amino acid sequence of SEQ ID NO: 10. where the hISL1 coding sequence is operably linked to expression control elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting glial cells to neurons in a subject in need thereof comprising: delivering an adeno-associated virus (AAV) to the subject in need thereof, where the AAV comprises a DNA vector construct comprising an insulin gene enhancer protein (ISL1) sequence, where the ISL1 sequence is operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) and a polyadenylation signal sequence, where the vector is capable of converting at least one glial cell to a neuron in the subject in need thereof.

In an aspect, this disclosure provides, and includes, a method of treating a neurological condition in a subject in need thereof comprising: delivering an adeno-associated virus (AAV) to the subject, where the AAV comprises a DNA vector construct comprising an insulin gene enhancer protein (ISL1) sequence, where the ISL1 sequence is operably linked to expression control elements comprising; (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal to the subject in need thereof.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a human LIM-homeobox 3 (hLHX3) sequence comprising the nucleic acid sequence of SEQ ID NO: 13, where the hLHX3 sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a nucleic acid coding sequence encoding a human LIM-homeobox 3 (hLHX3) protein comprising the amino acid sequence of SEQ ID NO: 14, where the hLHX3 coding sequence is operably linked to expression control elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting glial cells to neurons in a subject in need thereof comprising: delivering an adeno-associated virus (AAV) to the subject in need thereof, where the AAV comprises a DNA vector construct comprising a LIM-homeobox 3 (LHX3) sequence, where the LHX3 sequence is operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) and a polyadenylation signal sequence, where the vector is capable of converting at least one glial cell to a neuron in the subject in need thereof.

In an aspect, this disclosure provides, and includes, a method of treating a neurological condition in a subject in need thereof comprising: delivering an adeno-associated virus (AAV) to the subject, where the AAV comprises a DNA vector construct comprising a LIM-homeobox 3 (LHX3) sequence, where the LHX3 sequence is operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and; (e) a polyadenylation signal to the subject in need thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depict a map of a CE:GfaABC1D:CI:ISL1:P2A:LHX3:WPRE:SV40.

FIG. 1B depict a map of a EF-1α:GfaABC1D:CI:ISL1:P2A:LHX3:WPRE:SV40.

FIG. 1C depict a map of a CE:GfaABC1D:CI:ISL1:P2A:LHX3:WPRE:hGH.

FIG. 1D depict a map of a EF-1α:GfaABC1D:CI:ISL1:P2A:LHX3:WPRE:hGH.

FIG. 2A depict a map of a CE:GfaABC1D:CI:ISL1:GSG-P2A:LHX3:WPRE:SV40.

FIG. 2B depict a map of a EF-1α:GfaABC1D:CI:ISL1:GSG-P2A:LHX3:WPRE:SV40.

FIG. 2C depict a map of a CE:GfaABC1D:CI:ISL1:GSG-P2A:LHX3:WPRE:hGH.

FIG. 2D depict a map of a EF-1α:GfaABC1D:CI:ISL1:GSG-P2A:LHX3:WPRE:hGH.

FIG. 3A depict a map of a CE:GfaABC1D:CI:ISL1:T2A:LHX3:WPRE:SV40.

FIG. 3B depict a map of a EF-1α:GfaABC1D:CI:ISL1:T2A:LHX3:WPRE:SV40.

FIG. 3C depict a map of a CE:GfaABC1D:CI:ISL1:T2A:LHX3:WPRE:hGH.

FIG. 3D depict a map of a EF-1α:GfaABC1D:CI:ISL1 T2A:LHX3:WPRE:hGH.

FIG. 4A depict a map of a CE:GfaABC1D:CI:ISL1:GSG-T2A:LHX3:WPRE:SV40.

FIG. 4B depict a map of a EF-1α:GfaABC1D:CI:ISL1:GSG-T2A:LHX3:WPRE:SV40.

FIG. 4C depict a map of a CE:GfaABC1D:CI:ISL1:GSG-T2A:LHX3:WPRE:hGH.

FIG. 4D depict a map of a EF-1α:GfaABC1D:CI:ISL1:GSG-T2A:LHX3:WPRE:hGH.

FIG. 5A depicts a map of a CE:GfaABC1D:CI:ISL1:WPRE:SV40.

FIG. 5B depicts a map of a EF-1α:GfaABC1D:CI:ISL1:WPRE:SV40.

FIG. 5C depicts a map of a CE:Gfa1.6p:CI:ISL1:WPRE:SV40.

FIG. 5D depicts a map of a EF-1α:Gfa1.6p:CI:ISL1:WPRE:SV40

FIG. 5E depicts a map of a CE:Gfa2.2:CI:ISL1:WPRE:SV40.

FIG. 5F depicts a map of a EF-1α: Gfa2.2:CI:ISL1:WPRE:SV40.

FIG. 6A depicts a map of a CE:GfaABC1D:CI:ISL1:WPRE:hGH.

FIG. 6B depicts a map of a EF-1α:GfaABC1D:CI:ISL1:WPRE:hGH.

FIG. 6C depicts a map of a CE:Gfa1.6p:CI:ISL1:WPRE:hGH.

FIG. 6D depicts a map of a EF-1α:Gfa1.6p:CI:ISL1:WPRE:hGH

FIG. 6E depicts a map of a CE:Gfa2.2:CI:ISL1:WPRE:hGH.

FIG. 6F depicts a map of a EF-1α: Gfa2.2:CI:ISL1:WPRE:hGH.

FIG. 7A depicts a map of a CE:GfaABC1D:CI:LHX3:WPRE:SV40.

FIG. 7B depicts a map of a EF-1α:GfaABC1D:CI:LHX3:WPRE:SV40.

FIG. 7C depicts a map of a CE:Gfa1.6p:CI:LHX3:WPRE:SV40.

FIG. 7D depicts a map of a EF-1α:Gfa1.6p:CI:LHX3:WPRE:SV40

FIG. 7E depicts a map of a CE:Gfa2.2:CI:LHX3:WPRE:SV40.

FIG. 7F depicts a map of a EF-1α: Gfa2.2:CI:LHX3:WPRE:SV40.

FIG. 8A depicts a map of a CE:GfaABC1D:CI:LHX3:WPRE:hGH.

FIG. 8B depicts a map of a EF-1α:GfaABC1D:CI:LHX3:WPRE:hGH.

FIG. 8C depicts a map of a CE:Gfa1.6p:CI:LHX3:WPRE:hGH.

FIG. 8D depicts a map of a EF-1α:Gfa1.6p:CI:LHX3:WPRE:hGH

FIG. 8E depicts a map of a CE:Gfa2.2:CI:LHX3:WPRE:hGH.

FIG. 8F depicts a map of a EF-1α:Gfa2.2:CI:LHX3:WPRE:hGH.

FIG. 9 depicts Lec2 cells immunostained with an anti-Isl1 antibody and DAPI (nuclear stain) 24 hours post transfection with NXL-P141 (CE-pGfa681-CRGI-hIsl1-oPRE-bGHpA).

### BRIEF DESCRIPTION OF SEQUENCES

A listing of nucleic acid sequences and amino acid sequences is provided in Table 1.

TABLE 1
Nucleic acid sequences

| SEQ | | | |
|---|---|---|---|
| ID | Sequence | Sequence | |
| NO | Description | Type | Sequence |
| 1 | Upstream | Nucleic | TGCAGGCAGCTGCGCGCTCGCTCGCTCACTGAGGCCGC |
| | AAV2 ITR | acid | CCGGGCGTCGGGCGACCTTTGGTCGCCCGGCCTCAGTG |
| | | | AGCGAGCGAGCGCGCAGAGAGGGAGTGGCCAACTCCA |
| | | | TCACTAGGGGTTCCT |
| 2 | Ef1a | Nucleic | TGCAAAGATGGATAAAGTTTTAAACAGAGAGGAATCTT |
| | enhancer | acid | TGCAGCTAATGGACCTTCTAGGTCTTGAAAGGAGTGGG |
| | | | AATTGGCTCCGGTGCCCGTCAGTGGGCAGAGCGCACAT |
| | | | CGCCCACAGTCCCCGAGAAGTTGGGGGGAGGGGTCGGC |
| | | | A |
| 3 | GfaABC1D | Nucleic | CTAGTAACATATCCTGGTGTGGAGTAGGGGACGCTGCT |
| | promoter | acid | CTGACAGAGGCTCGGGGGCCTGAGCTGGCTCTGTGAGC |
| | (also called | | TGGGGAGGAGGCAGACAGCCAGGCCTTGTCTGCAAGCA |
| | “Gfa681” | | GACCTGGCAGCATTGGGCTGGCCGCCCCCCAGGGCCTC |
| | promoter) | | CTCTTCATGCCCAGTGAATGACTCACCTTGGCACAGACA |
| | | | CAATGTTCGGGGTGGGCACAGTGCCTGCTTCCCGCCGC |
| | | | ACCCCAGCCCCCCTCAAATGCCTTCCGAGAAGCCCATT |
| | | | GAGCAGGGGGCTTGCATTGCACCCCAGCCTGACAGCCT |
| | | | GGCATCTTGGGATAAAAGCAGCACAGCCCCCTAGGGGC |
| | | | TGCCCTTGCTGTGTGGCGCCACCGGCGGTGGAGAACAA |
| | | | GGCTCTATTCAGCCTGTGCCCAGGAAAGGGGATCAGGG |
| | | | GATGCCCAGGCATGGACAGTGGGTGGCAGGGGGGGAG |
| | | | AGGAGGGCTGTCTGCTTCCCAGAAGTCCAAGGACACAA |
| | | | ATGGGTGAGGGGAGAGCTCTCCCCATAGCTGGGCTGCG |
| | | | GCCCAACCCCACCCCCTCAGGCTATGCCAGGGGGTGTT |
| | | | GCCAGGGGCACCCGGGCATCGCCAGTCTAGCCCACTCC |
| | | | TTCATAAAGCCCTCGCATCCCAGGAGCGAGCAGAGCCA |
| | | | GAGCAGGTTGGAGAGGAGACGCATCACCTCCGCTGCTC |
| | | | GC |
| 4 | Gfa1.6 | Nucleic | CTGCAAGCAGACCTGGCAGCATTGGGCTGGCCGCCCCC |
| | promoter | acid | CAGGGCCTCCTCTTCATGCCCAGTGAATGACTCACCTTG |
| | | | GCACAGACACAATGTTCGGGGTGGGCACAGTGCCTGCT |
| | | | TCCCGCCGCACCCCAGCCCCCCTCAAATGCCTTCCGAGA |
| | | | AGCCCATTGAGTAGGGGGCTTGCATTGCACCCCAGCCT |
| | | | GACAGCCTGGCATCTTGGGATAAAAGCAGCACAGCCCC |
| | | | CTAGGGGCTGCCCTTGCTGTGTGGCGCCACCGGCGGTG |
| | | | GAGAACAAGGCTCTATTCAGCCTGTGCCCAGGAAAGGG |
| | | | GATCAGGGGATGCCCAGGCATGGACAGTGGGTGGCAG |
| | | | GGGGGGAGAGGAGGGCTGTCTGCTTCCCAGAAGTCCAA |
| | | | GGACACAAATGGGTGAGGGGACTGGGCAGGGTTCTGAC |
| | | | CCTGTGGGACCAGAGTGGAGGGCGTAGATGGACCTGAA |
| | | | GTCTCCAGGGACAACAGGGCCCAGGTCTCAGGCTCCTA |
| | | | GTTGGGCCCAGTGGCTCCAGCGTTTCCAAACCCATCCAT |
| | | | CCCCAGAGGTTCTTCCCATCTCTCCAGGCTGATGTGTGG |
| | | | GAACTCGAGGAAATAAATCTCCAGTGGGAGACGGAGG |
| | | | GGTGGCCAGGGAAACGGGGCGCTGCAGGAATAAAGAC |
| | | | GAGCCAGCACAGCCAGCTCATGCGTAACGGCTTTGTGG |
| | | | AGCTGTCAAGGCCTGGTCTCTGGGAGAGAGGCACAGGG |
| | | | AGGCCAGACAAGGAAGGGGTGACCTGGAGGGACAGAT |
| | | | CCAGGGGCTAAAGTCCTGATAAGGCAAGAGAGTGCCGG |
| | | | CCCCCTCTTGCCCTATCAGGACCTCCACTGCCACATAGA |
| | | | GGCCATGATTGACCCTTAGACAAAGGGCTGGTGTCCAA |
| | | | TCCCAGCCCCCAGCCCCAGAACTCCAGGGAATGAATGG |
| | | | GCAGAGAGCAGGAATGTGGGACATCTGTGTTCAAGGGA |
| | | | AGGACTCCAGGAGTCTGCTGGGAATGAGGCCTAGTAGG |
| | | | AAATGAGGTGGCCCTTGAGGGTACAGAACAGGTTCATT |
| | | | CTTCGCCAAATTCCCAGCACCTTGCAGGCACTTACAGCT |
| | | | GAGTGAGATAATGCCTGGGTTATGAAATCAAAAAGTTG |
| | | | GAAAGCAGGTCAGAGGTCATCTGGTACAGCCCTTCCTT |
| | | | CCCTTTTTTTTTTTTTTTTTTTGTGAGACAAGGTCTCTCT |
| | | | CTGTTGCCCAGGCTGGAGTGGCGCAAACACAGCTCACT |
| | | | GCAGCCTCAACCTACTGGGCTCAAGCAATCCTCCAGCC |
| | | | TCAGCCTCCCAAAGTGCTGGGATTACAAGCATGAGCCA |
| | | | CCCCACTCAGCCCTTTCCTTCCTTTTTAATTGATGCATAA |
| | | | TAATTGTAAGTATTCATCATGGTCCAACCAACCCTTTCT |
| | | | TGACCCACCTTCCTAGAGAGAGGGTCCTCTTGATTCAGC |
| | | | GGTCAGGGCCCCAGACCCATGGTCTGGCTCCAGGTACC |
| | | | ACCTGCCTCATGCAGGAGTTGGCGTGCCCAGGAAGCTC |
| | | | TGCCTCTGGGCACAGTGACCTCAGTGGGGTGAGGGGAG |
| | | | CTCTCCCCATAGCTGGGCTGCGGCCCAACCCCACCCCCT |
| | | | CAGGCTATGCCAGGGGGTGTTGCCAGGGGCACCCGGGC |
| | | | ATCGCCAGTCTAGCCCACTCCTTCATAAAGCCCTCGCAT |
| | | | CCCAGGAGCGAGCAGAGCCAGAG |
| 5 | Chimeric | Nucleic | GTAAGTATCAAGGTTACAAGACAGGTTTAAGGAGACCA |
| | Intron (CI) | acid | ATAGAAACTGGGCTTGTCGAGACAGAGAAGACTCTTGC |
| | | | GTTTCTGATAGGCACCTATTGGTCTTACTGACATCCACT |
| | | | TTGCCTTTCTCTCCACAG |
| 6 | human ISL1 | Nucleic | ATGGGAGACATGGGAGATCCACCAAAAAAAAAACGTC |
| | | acid | TGATTTCCCTATGTGTTGGTTGCGGCAATCAGATTCACG |
| | | | ATCAGTATATTCTGAGGGTTTCTCCGGATTTGGAATGGC |
| | | | ATGCGGCATGTTTGAAATGTGCGGAGTGTAATCAGTAT |
| | | | TTGGACGAGAGCTGTACATGCTTTGTTAGGGATGGGAA |
| | | | AACCTACTGTAAAAGAGATTATATCAGGTTGTACGGGA |
| | | | TCAAATGCGCCAAGTGCAGCATCGGCTTCAGCAAGAAC |
| | | | GACTTCGTGATGCGTGCCCGCTCCAAGGTGTATCACATC |
| | | | GAGTGTTTCCGCTGTGTGGCCTGCAGCCGCCAGCTCATC |
| | | | CCTGGGGACGAATTTGCGCTTCGGGAGGACGGTCTCTT |
| | | | CTGCCGAGCAGACCACGATGTGGTGGAGAGGGCCAGTC |
| | | | TAGGCGCTGGCGACCCGCTCAGTCCCCTGCATCCAGCG |
| | | | CGGCCACTGCAAATGGCAGCGGAGCCCATCTCCGCCAG |
| | | | GCAGCCGGCCCTGCGGCCCCACGTCCACAAGCAGCCGG |
| | | | AGAAGACCACCCGCGTGCGGACTGTGCTGAACGAGAAG |
| | | | CAGCTGCACACCTTGCGGACCTGCTACGCCGCAAACCC |
| | | | GCGGCCAGATGCGCTCATGAAGGAGCAACTGGTAGAGA |
| | | | TGACGGGCCTCAGTCCCCGTGTGATCCGGGTCTGGTTTC |
| | | | AAAACAAGCGGTGCAAGGACAAGAAGCGAAGCATCAT |
| | | | GATGAAGCAACTCCAGCAGCAGCAGCCCAATGACAAA |
| | | | ACTAATATCCAGGGGATGACAGGAACTCCCATGGTGGC |
| | | | TGCCAGTCCAGAGAGACACGACGGTGGCTTACAGGCTA |
| | | | ACCCAGTGGAAGTACAAAGTTACCAGCCACCTTGGAAA |
| | | | GTACTGAGCGACTTCGCCTTGCAGAGTGACATAGATCA |
| | | | GCCTGCTTTTCAGCAACTGGTCAATTTTTCAGAAGGAGG |
| | | | ACCGGGCTCTAATTCCACTGGCAGTGAAGTAGCATCAA |
| | | | TGTCCTCTCAACTTCCAGATACACCTAACAGCATGGTAG |
| | | | CCAGTCCTATTGAGGCATGA |
| 7 | WPRE | Nucleic | AATCAACCTCTGGATTACAAAATTTGTGAAAGATTGAC |
| | (Woodchuck | acid | TGGTATTCTTAACTATGTTGCTCCTTTTACGCTATGTGG |
| | Hepatitis | | ATACGCTGCTTTAATGCCTTTGTATCATGCTATTGCTTCC |
| | Virus | | CGTATGGCTTTCATTTTCTCCTCCTTGTATAAATCCTGGT |
| | Posttranscriptional | | TGCTGTCTCTTTATGAGGAGTTGTGGCCCGTTGTCAGGC |
| | Regulatory | | AACGTGGCGTGGTGTGCACTGTGTTTGCTGACGCAACC |
| | Element) | | CCCACTGGTTGGGGCATTGCCACCACCTGTCAGCTCCTT |
| | | | TCCGGGACTTTCGCTTTCCCCCTCCCTATTGCCACGGCG |
| | | | GAACTCATCGCCGCCTGCCTTGCCCGCTGCTGGACAGG |
| | | | GGCTCGGCTGTTGGGCACTGACAATTCCGTGGTGTTGTC |
| | | | GGGGAAATCATCGTCCTTTCCTTGGCTGCTCGCCTGTGT |
| | | | TGCCACCTGGATTCTGCGCGGGACGTCCTTCTGCTACGT |
| | | | CCCTTCGGCCCTCAATCCAGCGGACCTTCCTTCCCGCGG |
| | | | CCTGCTGCCGGCTCTGCGGCCTCTTCCGCGTCTTCGCCT |
| | | | TCGCCCTCAGACGAGTCGGATCTCCCTTTGGGCCGCCTC |
| | | | CCCGC |
| 8 | SV40 | Nucleic | CGATCCACCGGATCTAGATAACTGATCATAATCAGCCA |
| | poly(A) | acid | TACCACATTTGTAGAGGTTTTACTTGCTTTAAAAAACCT |
| | signal | | CCCACACCTCCCCCTGAACCTGAAACATAAAATGAATG |
| | | | CAATTGTTGTTGTTAACTTGTTTATTGCAGCTTATAATG |
| | | | GTTACAAATAAAGCAATAGCATCACAAATTTCACAAAT |
| | | | AAAGCATTTTTTTCACTGCATTCTAGTTGTGGTTTGTCC |
| | | | AAACTCATCAATGTATCTTA |
| 9 | Downstream | Nucleic | AGGAACCCCTAGTGATGGAGTTGGCCACTCCCTCTCTGC |
| | AAV2 ITR | acid | GCGCTCGCTCGCTCACTGAGGCCGGGCGACCAAAGGTC |
| | | | GCCCGACGCCCGGGCTTTGCCCGGGCGGCCTCAGTGAG |
| | | | CGAGCGAGCGCGCAGCTGCCTGCA |
| 10 | human ISL1 | Amino | MGDMGDPPKKKRLISLCVGCGNQIHDQYILRVSPDLEWH |
| | | Acid | AACLKCAECNQYLDESCTCFVRDGKTYCKRDYIRLYGIKC |
| | | | AKCSIGFSKNDFVMRARSKVYHIECFRCVACSRQLIPGDEF |
| | | | ALREDGLFCRADHDVVERASLGAGDPLSPLHPARPLQMA |
| | | | AEPISARQPALRPHVHKQPEKTTRVRTVLNEKQLHTLRTC |
| | | | YAANPRPDALMKEQLVEMTGLSPRVIRVWFQNKRCKDK |
| | | | KRSIIVIMKQLQQQQPNDKTNIQGMTGTPMVAASPERHDG |
| | | | GLQANPVEVQSYQPPWKVLSDFALQSDIDQPAFQQLVNFS |
| | | | EGGPGSNSTGSEVASMSSQLPDTPNSMVASPIEA |
| 11 | CMV | Nucleic | GACATTGATTATTGACTAGTTATTAATAGTAATCAATTA |
| | enhancer | Acid | CGGGGTCATTAGTTCATAGCCCATATATGGAGTTCCGCG |
| | (CE) | | TTACATAACTTACGGTAAATGGCCCGCCTGGCTGACCG |
| | | | CCCAACGACCCCCGCCCATTGACGTCAATAATGACGTA |
| | | | TGTTCCCATAGTAACGCCAATAGGGACTTTCCATTGACG |
| | | | TCAATGGGTGGACTATTTACGGTAAACTGCCCACTTGGC |
| | | | AGTACATCAAGTGTATCATATGCCAAGTACGCCCCCTAT |
| | | | TGACGTCAATGACGGTAAATGGCCCGCCTGGCATTATG |
| | | | CCCAGTACATGACCTTATGGGACTTTCCTACTTGGCAGT |
| | | | ACATCTACGTATTAGTCATCGCTATTACCATG |
| 12 | hGFA2.2 | Nucleic | CGCGTCCCACCTCCCTCTCTGTGCTGGGACTCACAGAGG |
| | promoter | Acid | GAGACCTCAGGAGGCAGTCTGTCCATCACATGTCCAAA |
| | | | TGCAGAGCATACCCTGGGCTGGGCGCAGTGGCGCACAA |
| | | | CTGTAATTCCAGCACTTTGGGAGGCTGATGTGGAAGGA |
| | | | TCACTTGAGCCCAGAAGTTCTAGACCAGCCTGGGCAAC |
| | | | ATGGCAAGACCCTATCTCTACAAAAAAAGTTAAAAAAT |
| | | | CAGCCACGTGTGGTGACACACACCTGTAGTCCCAGCTA |
| | | | TTCAGGAGGCTGAGGTGAGGGGATCACTTAAGGCTGGG |
| | | | AGGTTGAGGCTGCAGTGAGTCGTGGTTGCGCCACTGCA |
| | | | CTCCAGCCTGGGCAACAGTGAGACCCTGTCTCAAAAGA |
| | | | CAAAAAAAAAAAAAAAAAAAAAAAGAACATATCCTGG |
| | | | TGTGGAGTAGGGGACGCTGCTCTGACAGAGGCTCGGGG |
| | | | GCCTGAGCTGGCTCTGTGAGCTGGGGAGGAGGCAGACA |
| | | | GCCAGGCCTTGTCTGCAAGCAGACCTGGCAGCATTGGG |
| | | | CTGGCCGCCCCCCAGGGCCTCCTCTTCATGCCCAGTGAA |
| | | | TGACTCACCTTGGCACAGACACAATGTTCGGGGTGGGC |
| | | | ACAGTGCCTGCTTCCCGCCGCACCCCAGCCCCCCTCAAA |
| | | | TGCCTTCCGAGAAGCCCATTGAGCAGGGGGCTTGCATT |
| | | | GCACCCCAGCCTGACAGCCTGGCATCTTGGGATAAAAG |
| | | | CAGCACAGCCCCCTAGGGGCTGCCCTTGCTGTGTGGCG |
| | | | CCACCGGCGGTGGAGAACAAGGCTCTATTCAGCCTGTG |
| | | | CCCAGGAAAGGGGATCAGGGGATGCCCAGGCATGGAC |
| | | | AGTGGGTGGCAGGGGGGGAGAGGAGGGCTGTCTGCTTC |
| | | | CCAGAAGTCCAAGGACACAAATGGGTGAGGGGACTGG |
| | | | GCAGGGTTCTGACCCTGTGGGACCAGAGTGGAGGGCGT |
| | | | AGATGGACCTGAAGTCTCCAGGGACAACAGGGCCCAGG |
| | | | TCTCAGGCTCCTAGTTGGGCCCAGTGGCTCCAGCGTTTC |
| | | | CAAACCCATCCATCCCCAGAGGTTCTTCCCATCTCTCCA |
| | | | GGCTGATGTGTGGGAACTCGAGGAAATAAATCTCCAGT |
| | | | GGGAGACGGAGGGGTGGCCAGGGAAACGGGGCGCTGC |
| | | | AGGAATAAAGACGAGCCAGCACAGCCAGCTCATGTGTA |
| | | | ACGGCTTTGTGGAGCTGTCAAGGCCTGGTCTCTGGGAG |
| | | | AGAGGCACAGGGAGGCCAGACAAGGAAGGGGTGACCT |
| | | | GGAGGGACAGATCCAGGGGCTAAAGTCCTGATAAGGC |
| | | | AAGAGAGTGCCGGCCCCCTCTTGCCCTATCAGGACCTC |
| | | | CACTGCCACATAGAGGCCATGATTGACCCTTAGACAAA |
| | | | GGGCTGGTGTCCAATCCCAGCCCCCAGCCCCAGAACTC |
| | | | CAGGGAATGAATGGGCAGAGAGCAGGAATGTGGGACA |
| | | | TCTGTGTTCAAGGGAAGGACTCCAGGAGTCTGCTGGGA |
| | | | ATGAGGCCTAGTAGGAAATGAGGTGGCCCTTGAGGGTA |
| | | | CAGAACAGGTTCATTCTTCGCCAAATTCCCAGCACCTTG |
| | | | CAGGCACTTACAGCTGAGTGAGATAATGCCTGGGTTAT |
| | | | GAAATCAAAAAGTTGGAAAGCAGGTCAGAGGTCATCTG |
| | | | GTACAGCCCTTCCTTCCCTTTTTTTTTTTTTTTTTTTGTGA |
| | | | GACAAGGTCTCTCTCTGTTGCCCAGGCTGGAGTGGCGC |
| | | | AAACACAGCTCACTGCAGCCTCAACCTACTGGGCTCAA |
| | | | GCAATCCTCCAGCCTCAGCCTCCCAAAGTGCTGGGATT |
| | | | ACAAGCATGAGCCACCCCACTCAGCCCTTTCCTTCCTTT |
| | | | TTAATTGATGCATAATAATTGTAAGTATTCATCATGGTC |
| | | | CAACCAACCCTTTCTTGACCCACCTTCCTAGAGAGAGG |
| | | | GTCCTCTTGCTTCAGCGGTCAGGGCCCCAGACCCATGGT |
| | | | CTGGCTCCAGGTACCACCTGCCTCATGCAGGAGTTGGC |
| | | | GTGCCCAGGAAGCTCTGCCTCTGGGCACAGTGACCTCA |
| | | | GTGGGGTGAGGGGAGCTCTCCCCATAGCTGGGCTGCGG |
| | | | CCCAACCCCACCCCCTCAGGCTATGCCAGGGGGTGTTG |
| | | | CCAGGGGCACCCGGGCATCGCCAGTCTAGCCCACTCCT |
| | | | TCATAAAGCCCTCGCATCCCAGGAGCGAGCAGAGCCAG |
| | | | AGCAGGTTGGAGAGGAGACGCATCACCTCCGCTGCTCG |
| | | | CCGGG |
| 13 | Human | Nucleic | ATGGAGGCGCGCGGGGAGCTGGGCCCGGCCCGGGAGT |
| | LHX3 | Acid | CGGCGGGAGGCGACCTGCTGCTAGCACTGCTGGCGCGG |
| | | | AGGGCGGACCTGCGCCGAGAGATCCCGCTGTGCGCTGG |
| | | | CTGTGACCAGCACATCCTGGACCGCTTCATCCTCAAGGC |
| | | | TCTGGACCGCCACTGGCACAGCAAGTGTCTCAAGTGCA |
| | | | GCGACTGCCACACGCCACTGGCCGAGCGCTGCTTCAGC |
| | | | CGAGGGGAGAGCGTTTACTGCAAGGACGACTTTTTCAA |
| | | | GCGCTTCGGGACCAAGTGCGCCGCGTGCCAGCTGGGCA |
| | | | TCCCGCCCACGCAGGTGGTGCGCCGCGCCCAGGACTTC |
| | | | GTGTACCACCTGCACTGCTTTGCCTGCGTCGTGTGCAAG |
| | | | CGGCAGCTGGCCACGGGCGACGAGTTCTACCTCATGGA |
| | | | GGACAGCCGGCTCGTGTGCAAGGCGGACTACGAAACCG |
| | | | CCAAGCAGCGAGAGGCCGAGGCCACGGCCAAGCGGCC |
| | | | GCGCACGACCATCACCGCCAAGCAGCTGGAGACGCTGA |
| | | | AGAGCGCTTACAACACCTCGCCCAAGCCGGCGCGCCAC |
| | | | GTGCGCGAGCAGCTCTCGTCCGAGACGGGCCTGGACAT |
| | | | GCGCGTGGTGCAGGTTTGGTTCCAGAACCGCCGGGCCA |
| | | | AGGAGAAGAGGCTGAAGAAGGACGCCGGCCGGCAGCG |
| | | | CTGGGGGCAGTATTTCCGCAACATGAAGCGCTCCCGCG |
| | | | GCGGCTCCAAGTCGGACAAGGACAGCGTTCAGGAGGG |
| | | | GCAGGACAGCGACGCTGAGGTCTCCTTCCCCGATGAGC |
| | | | CTTCCTTGGCGGAAATGGGCCCGGCCAATGGCCTCTAC |
| | | | GGGAGCTTGGGGGAACCCACCCAGGCCTTGGGCCGGCC |
| | | | CTCGGGAGCCCTGGGCAACTTCTCCCTGGAGCATGGAG |
| | | | GCCTGGCAGGCCCAGAGCAGTACCGAGAGCTGCGTCCC |
| | | | GGCAGCCCCTACGGTGTCCCCCCATCCCCCGCCGCCCCG |
| | | | CAGAGCCTCCCTGGCCCCCAGCCCCTCCTCTCCAGCCTG |
| | | | GTGTACCCAGACACCAGCTTGGGCCTTGTGCCCTCGGG |
| | | | AGCCCCCGGCGGGCCCCCACCCATGAGGGTGCTGGCAG |
| | | | GGAACGGACCCAGTTCTGACCTATCCACGGGGAGCAGC |
| | | | GGGGGTTACCCCGACTTCCCTGCCAGCCCCGCCTCCTGG |
| | | | CTGGATGAGGTAGACCACGCTCAGTTCTGA |
| 14 | Human | Amino | MEARGELGPARESAGGDLLLALLARRADLRREIPLCAGCD |
| | LHX3 | Acid | QHILDRFILKALDRHWHSKCLKCSDCHTPLAERCFSRGES |
| | | | VYCKDDFFKRFGTKCAACQLGIPPTQVVRRAQDFVYHLH |
| | | | CFACVVCKRQLATGDEFYLMEDSRLVCKADYETAKQREA |
| | | | EATAKRPRTTITAKQLETLKSAYNTSPKPARHVREQLSSET |
| | | | GLDMRVVQVWFQNRRAKEKRLKKDAGRQRWGQYFRNM |
| | | | KRSRGGSKSDKDSVQEGQDSDAEVSFPDEPSLAEMGPAN |
| | | | GLYGSLGEPTQALGRPSGALGNFSLEHGGLAGPEQYRELR |
| | | | PGSPYGVPPSPAAPQSLPGPQPLLSSLVYPDTSLGLVPSG |
| | | | APGGPPPMRVLAGNGPSSDLSTGSSGGYPDFPASPASWLD |
| | | | EVDHAQF |
| 15 | P2A | Nucleic | GCTACTAACTTCAGCCTGCTGAAGCAGGCTGGAGACGT |
| | | Acid | GGAGGAGAACCCTGGACCT |
| 16 | T2A | Nucleic | GAGGGGAGAGGAAGTCTTCTGACCTGCGGAGACGTCGA |
| | | Acid | AGAGAATCCTGGACCC |
| 17 | hGH poly(A) | Nucleic | GGGTGGCATCCCTGTGACCCCTCCCCAGTGCCTCTCCTG |
| | signal | Acid | GCCCTGGAAGTTGCCACTCCAGTGCCCACCAGCCTTGTC |
| | | | CTAATAAAATTAAGTTGCATCATTTTGTCTGACTAGGTG |
| | | | TCCTTCTATAATATTATGGGGTGGAGGGGGGTGGTATG |
| | | | GAGCAAGGGGCAAGTTGGGAAGACAACCTGTAGGGCC |
| | | | TGCGGGGTCTATTGGGAACCAAGCTGGAGTGCAGTGGC |
| | | | ACAATCTTGGCTCACTGCAATCTCCGCCTCCTGGGTTCA |
| | | | AGCGATTCTCCTGCCTCAGCCTCCCGAGTTGTTGGGATT |
| | | | CCAGGCATGCATGACCAGGCTCAGCTAATTTTTGTTTTT |
| | | | TTGGTAGAGACGGGGTTTCACCATATTGGCCAGGCTGG |
| | | | TCTCCAACTCCTAATCTCAGGTGATCTACCCACCTTGGC |
| | | | CTCCCAAATTGCTGGGATTACAGGCGTGAACCACTGCT |
| | | | CCCTTCCCTGTCCTT |
| 18 | GSG-P2A | Nucleic | GGAAGCGGAGCTACTAACTTCAGCCTGCTGAAGCAGGC |
| | | Acid | TGGAGACGTGGAGGAGAACCCTGGACCT |
| 19 | GSG-T2A | Nucleic | GGAAGCGGAGCTACTAACTTCAGCCTGCTGAAGCAGGC |
| | | Acid | TGGAGACGTGGAGGAGAACCCTGGACCT |
| 20 | GSG-P2A | Amino | GSGATNFSLLKQAGDVEENPGP |
| | | Acid | |
| 21 | GSG-T2A | Amino | GSGEGRGSLLTCGDVEENPGP |
| | | Acid | |
| 22 | CRGI | Nucleic | GGAGTCGCTGCGTTGCCTTCGCCCCGTGCCCCGCTCCGC |
| | Chimeric | Acid | GCCGCCTCGCGCCGCCCGCCCCGGCTCTGACTGACCGC |
| | Intron | | GTTACTCCCACAGGTGAGCGGGCGGGACGGCCCTTCTC |
| | | | CCTCCGGGCTGTAATTAGCGCTTGGTTTAATGACGGCTC |
| | | | GTTTCTTTTCTGTGGCTGCGTGAAAGCCTTAAAGGGCTC |
| | | | CGGGAGGGCCTTTGTGCGGGGGGGAGCGGCTCGGGGG |
| | | | GTGCGTGCGTGTGTGTGTGCGTGGGGAGCGCCGCGTGC |
| | | | GGCCCGCGCTGCCCGGCGGCTGTGAGCGCTGCGGGCGC |
| | | | GGCGCGGGGCTTTGTGCGCTCCGCGTGTGCGCGAGGGG |
| | | | AGCGCGGGCCGGGGGCGGTGCCCCGCGGTGCGGGGGG |
| | | | GCTGCGAGGGGAACAAAGGCTGCGTGCGGGGTGTGTGC |
| | | | GTGGGGGGGTGAGCAGGGGGTGTGGGCGCGGCGGTCG |
| | | | GGCTGTAACCCCCCCCTGGCACCCCCCTCCCCGAGTTGC |
| | | | TGAGCACGGCCCGGCTTCGGGTGCGGGGCTCCGTGCGG |
| | | | GGCGTGGCGCGGGGCTCGCCGTGCCGGGCGGGGGGTGG |
| | | | CGGCAGGTGGGGGTGCCGGGCGGGGCGGGGCCGCCTC |
| | | | GGGCCGGGGAGGGCTCGGGGGAGGGGCGCGGCGGCCC |
| | | | CGGAGCGCCGGCGGCTGTCGAGGCGCGGCGAGCCGCA |
| | | | GCCATTGCCTTTTATGGTAATCGTGCGAGAGGGCGCAG |
| | | | GGACTTCCTTTGTCCCAAATCTGGCGGAGCCGAAATCTG |
| | | | GGAGGCGCCGCCGCACCCCCTCTAGCGGGCGCGGGCGA |
| | | | AGCGGTGCGGCGCCGGCAGGAAGGAAATGGGCGGGGA |
| | | | GGGCCTTCGTGCGTCGCCGCGCCGCCGTCCCCTTCTCCA |
| | | | TCTCCAGCCTCGGGGCTGCCGCAGGGGGACGGCTGCCT |
| | | | TCGGGGGGGACGGGGCAGGGCGGGGTTCGGCTTCTGGC |
| | | | GTGTGACCGGCGGCTTTAGAGCCTCTGCTAACCATGTTC |
| | | | ATGCCTTCTTCTTTTTCCTACAGCTCCTGGGCAACGTGC |
| | | | TGGTTGTTGTGCTGTCTCATCATTTTGGCAAAGAT |
| 23 | Optimized | Nucleic | GAGCATCTTACCGCCATTTATACCCATATTTGTTCTGTTT |
| | version of | Acid | TTCTTGATTTGGGTATACATTTAAATGTTAATAAAACAA |
| | WPRE | | AATGGTGGGGCAATCATTTACATTTTTAGGGATATGTAA |
| | (oPRE) | | TTACTAGTTCAGGTGTATTGCCACAAGACAAACATGTTA |
| | | | AGAAACTTTCCCGTTATTTACGCTCTGTTCCTGTTAATC |
| | | | AACCTCTGGATTACAAAATTTGTGAAAGATTGACTGAT |
| | | | ATTCTTAACTATGTTGCTCCTTTTACGCTGTGTGGATAT |
| | | | GCTGCTTTATAGCCTCTGTATCTAGCTATTGCTTCCCGT |
| | | | ACGGCTTTCGTTTTCTCCTCCTTGTATAAATCCTGGTTGC |
| | | | TGTCTCTTTTAGAGGAGTTGTGGCCCGTTGTCCGTCAAC |
| | | | GTGGCGTGGTGTGCTCTGTGTTTGCTGACGCAACCCCCA |
| | | | CTGGCTGGGGCATTGCCACCACCTGTCAACTCCTTTCTG |
| | | | GGACTTTCGCTTTCCCCCTCCCGATCGCCACGGCAGAAC |
| | | | TCATCGCCGCCTGCCTTGCCCGCTGCTGGACAGGGGCTA |
| | | | GGTTGCTGGGCACTGATAATTCCGTGGTGTTGTC |
| 24 | bGH poly | Nucleic | CTGTGCCTTCTAGTTGCCAGCCATCTGTTGTTTGCCCCTC |
| | (A) signal | Acid | CCCCGTGCCTTCCTTGACCCTGGAAGGTGCCACTCCCAC |
| | (bGHpA) | | TGTCCTTTCCTAATAAAATGAGGAAATTGCATCGCATTG |
| | | | TCTGAGTAGGTGTCATTCTATTCTGGGGGGTGGGGTGG |
| | | | GGCAGGACAGCAAGGGGGAGGATTGGGAAGAGAATAG |
| | | | CAGGCATGCTGGGGA |
| 25 | Synthetic | Nucleic | CACACAAAAAACCAACACACATCCATCTTCGATGGATA |
| | Poly(A) | Acid | GCGATTTTATT |
| | signal (SpA) | | |

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to be inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

As used herein “CE” refers to a cytomegalovirus (CMV) promoter enhancer sequence.

As used herein “EF-1α” refers to an Ef1 alpha promoter enhancer sequence.

As used herein “pGfa681” refers to a human glial fibrillary acid protein (GFAP) promoter truncated sequence of 681 bp size. As used herein “pGfa681,” “Gfa681,” “pGfaABC1D,” and “GfaABC1D” are used interchangeably.

As used herein “CI” refers to a chimeric intron composed of the 5′-donor site from the first intron of the human β-globin gene and the branch and 3′-acceptor site from the intron of an immunoglobulin gene heavy chain variable region.

As used herein “CRGI” refers to a chimeric intron of rabbit beta-globing and chicken beta actin similar in CAG promoter.

As used herein “WPRE” refers to a Woodchuck Hepatitis Virus (WHV) Posttranscriptional Regulatory Element.

As used herein “oPRE” refers to an optimized version of WPRE.

As used herein “SV40 pA” refers to a poly A signal of SV40 virus. As used herein “SV40 pA” and “SV40” are used interchangeably.

As used herein “bGHpA” refers to a poly A signal of bovine growth hormone. As used herein “bGHpA” and “bGH” are used interchangeably.

As used herein “hGH” refers to a poly A signal of human growth hormone. As used herein “hGHpA” and “hGH” are used interchangeably.

As used herein “SpA” refers to a synthetic poly A signal sequence.

As used herein “vg” refers to a viral genome.

As used herein “p2A” refers to a 2A self-cleavage peptide sequence from porcine teschovirus-1.

As used here “hls1” refers to a human insulin gene enhancer protein ISL-1.

Any composition or vector provided herein is specifically envisioned for use with any method provided herein.

In an aspect, methods and compositions provided herein comprise a vector. As used herein, the term “vector” refers to a circular, double-stranded DNA molecule that is physically separate from chromosomal DNA. It should be noted that the term “vector” can be used interchangeably with the term “plasmid.”

In an aspect, a vector provided herein is a recombinant vector. As used herein, the term “recombinant vector” refers to a vector that comprises a recombinant nucleic acid. As used herein, a “recombinant nucleic acid” refers to a nucleic acid molecule formed by laboratory methods of genetic recombination, such as, without being limiting, molecular cloning. A recombinant vector can be formed by laboratory methods of genetic recombination, such as, without being limiting, molecular cloning. Also, without being limiting, one skilled in the art can create a recombinant vector de novo via synthesizing a plasmid by individual nucleotides, or by splicing together nucleic acid molecules from different pre-existing vectors.

Adeno-associated viruses (AAVs) are replication-defective, non-enveloped Dependoparvovirus viruses that infect humans and additional primate species. AAVs are not known to cause disease in any species, although they can cause mild immune responses. AAVs can infect dividing and quiescent cells. AAVs are stably integrate into the human genome at a specific site in chromosome 19 termed the AAVS1 locus (nucleotides 7774-11429 of GenBank Accession No. AC010327.8), although random integrations at other loci in the human genome are possible.

AAVs comprise a linear genome with a single-stranded DNA of about 4700 nucleotides in length. The genome of AAVs also includes a 145 nucleotide-long inverted terminal repeat (ITR) at each end of the genome. The ITRs flank two viral genes rep (for replication, encoding non-structural proteins) and cap (for capsid, encoding structural proteins). The ITRs contain all of the cis-acting elements need for genome rescue, replication, and packaging of the AAV.

When used in gene therapy approaches, the rep and cap genes of the AAV genome sequence are removed and replaced with DNA of interest positioned between two AAV ITRs. As used herein, an “AAV vector construct” refers to a DNA molecule comprising a desired sequence inserted between two AAV ITR sequences. As used herein, an “AAV vector” refers to an AAV packaged with a DNA vector construct.

As used herein, the term “AAV vector serotype” mainly refers to a variation within the capsid proteins of an AAV vector.

In an aspect, an AAV vector is selected from the group consisting of AAV vector serotype 1, AAV vector serotype 2, AAV vector serotype 3, AAV vector serotype 4, AAV vector serotype 5, AAV vector serotype 6, AAV vector serotype 7, AAV vector serotype 8, AAV vector serotype 9, AAV vector serotype 10, AAV vector serotype 11, and AAV vector serotype 12. In one aspect, an AAV vector is selected from the group consisting AAV serotype 2, AAV serotype 5, and AAV serotype 9. In one aspect, an AAV vector is AAV serotype 1. In one aspect, an AAV vector is AAV serotype 2. In one aspect, an AAV vector is AAV serotype 3. In one aspect, an AAV vector is AAV serotype 4. In one aspect, an AAV vector is AAV serotype 5. In one aspect, an AAV vector is AAV serotype 6. In one aspect, an AAV vector is AAV serotype 7. In one aspect, an AAV vector is AAV serotype 8. In one aspect, an AAV vector is AAV serotype 9. In one aspect, an AAV vector is AAV serotype 10. In one aspect, an AAV vector is AAV serotype 11. In one aspect, an AAV vector is AAV serotype 12.

In an aspect, an AAV vector ITR is selected from the group consisting of an AAV serotype 1 ITR, an AAV serotype 2 ITR, an AAV serotype 3 ITR, an AAV serotype 4 ITR, an AAV serotype 5 ITR, an AAV serotype 6 ITR, an AAV serotype 7 ITR, an AAV serotype 8 ITR, an AAV serotype 9 ITR, an AAV serotype 10 ITR, an AAV serotype 11 ITR, and an AAV serotype 12 ITR. In one aspect, an AAV vector ITR is an AAV serotype 1 ITR. In one aspect, an AAV vector ITR is an AAV serotype 2 ITR. In one aspect, an AAV vector ITR is an AAV serotype 3 ITR. In one aspect, an AAV vector ITR is an AAV serotype 4 ITR. In one aspect, an AAV vector ITR is an AAV serotype 5 ITR. In one aspect, an AAV vector ITR is an AAV serotype 6 ITR. In one aspect, an AAV vector ITR is an AAV serotype 7 ITR. In one aspect, an AAV vector ITR is an AAV serotype 8 ITR. In one aspect, an AAV vector ITR is an AAV serotype 9 ITR. In one aspect, an AAV vector ITR is an AAV serotype 10 ITR. In one aspect, an AAV vector ITR is an AAV serotype 11 ITR. In one aspect, an AAV vector ITR is an AAV serotype 12 ITR.

In an aspect, at least one AAV vector ITR nucleic acid sequence is selected from the group consisting of SEQ ID NO: 1 and 9. In one aspect, at least one AAV vector ITR nucleic acid sequence is SEQ ID NO 1. In one aspect, at least one AAV vector ITR nucleic acid sequence is SEQ ID NO 9.

In an aspect, an AAV ITR nucleic acid sequence comprises a sequence at least 70% identical to SEQ ID NO: 1, or the complement thereof. In one aspect, an AAV ITR nucleic acid sequence comprises a sequence at least 75% identical to SEQ ID NO: 1, or the complement thereof.

In one aspect, an AAV ITR nucleic acid sequence comprises a sequence at least 80% identical to SEQ ID NO: 1, or the complement thereof. In one aspect, an AAV ITR nucleic acid sequence comprises a sequence at least 85% identical to SEQ ID NO: 1, or the complement thereof. In one aspect, an AAV ITR nucleic acid sequence comprises a sequence at least 90% identical to SEQ ID NO: 1, or the complement thereof. In one aspect, an AAV ITR nucleic acid sequence comprises a sequence at least 91% identical to SEQ ID NO: 1, or the complement thereof. In one aspect, an AAV ITR nucleic acid sequence comprises a sequence at least 92% identical to SEQ ID NO: 1, or the complement thereof. In one aspect, an AAV ITR nucleic acid sequence comprises a sequence at least 93% identical to SEQ ID NO: 1, or the complement thereof. In one aspect, an AAV ITR nucleic acid sequence comprises a sequence at least 94% identical to SEQ ID NO: 1, or the complement thereof. In one aspect, an AAV ITR nucleic acid sequence comprises a sequence at least 95% identical to SEQ ID NO: 1, or the complement thereof. In one aspect, an AAV ITR nucleic acid sequence comprises a sequence at least 96% identical to SEQ ID NO: 1, or the complement thereof. In one aspect, an AAV ITR nucleic acid sequence comprises a sequence at least 97% identical to SEQ ID NO: 1, or the complement thereof. In one aspect, an AAV ITR nucleic acid sequence comprises a sequence at least 98% identical to SEQ ID NO: 1, or the complement thereof. In one aspect, an AAV ITR nucleic acid sequence comprises a sequence at least 99% identical to SEQ ID NO: 1, or the complement thereof. In one aspect, an AAV ITR nucleic acid sequence comprises a sequence at least 99.5% identical to SEQ ID NO: 1, or the complement thereof. In one aspect, an AAV ITR nucleic acid sequence comprises a sequence at least 99.8% identical to SEQ ID NO: 1, or the complement thereof. In one aspect, an AAV ITR nucleic acid sequence comprises a sequence at least 99.9% identical to SEQ ID NO: 1, or the complement thereof. In one aspect, an AAV ITR nucleic acid sequence comprises a sequence 100% identical to SEQ ID NO: 1, or the complement thereof.

In an aspect, an AAV ITR nucleic acid sequence comprises a sequence at least 70% identical to SEQ ID NO: 9, or the complement thereof. In one aspect, an AAV ITR nucleic acid sequence comprises a sequence at least 75% identical to SEQ ID NO: 9, or the complement thereof. In one aspect, an AAV ITR nucleic acid sequence comprises a sequence at least 80% identical to SEQ ID NO: 9, or the complement thereof. In one aspect, an AAV ITR nucleic acid sequence comprises a sequence at least 85% identical to SEQ ID NO: 9, or the complement thereof. In one aspect, an AAV ITR nucleic acid sequence comprises a sequence at least 90% identical to SEQ ID NO: 9, or the complement thereof. In one aspect, an AAV ITR nucleic acid sequence comprises a sequence at least 91% identical to SEQ ID NO: 9, or the complement thereof. In one aspect, an AAV ITR nucleic acid sequence comprises a sequence at least 92% identical to SEQ ID NO: 9, or the complement thereof. In one aspect, an AAV ITR nucleic acid sequence comprises a sequence at least 93% identical to SEQ ID NO: 9, or the complement thereof. In one aspect, an AAV ITR nucleic acid sequence comprises a sequence at least 94% identical to SEQ ID NO: 9, or the complement thereof. In one aspect, an AAV ITR nucleic acid sequence comprises a sequence at least 95% identical to SEQ ID NO: 9, or the complement thereof. In one aspect, an AAV ITR nucleic acid sequence comprises a sequence at least 96% identical to SEQ ID NO: 9, or the complement thereof. In one aspect, an AAV ITR nucleic acid sequence comprises a sequence at least 97% identical to SEQ ID NO: 9, or the complement thereof. In one aspect, an AAV ITR nucleic acid sequence comprises a sequence at least 98% identical to SEQ ID NO: 9, or the complement thereof. In one aspect, an AAV ITR nucleic acid sequence comprises a sequence at least 99% identical to SEQ ID NO: 9, or the complement thereof. In one aspect, an AAV ITR nucleic acid sequence comprises a sequence at least 99.5% identical to SEQ ID NO: 9, or the complement thereof. In one aspect, an AAV ITR nucleic acid sequence comprises a sequence at least 99.8% identical to SEQ ID NO: 9, or the complement thereof. In one aspect, an AAV ITR nucleic acid sequence comprises a sequence at least 99.9% identical to SEQ ID NO: 9, or the complement thereof. In one aspect, an AAV ITR nucleic acid sequence comprises a sequence 100% identical to SEQ ID NO: 9, or the complement thereof.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31: 3497-3500 (2003); Thompson et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

The use of the term “polynucleotide,” “nucleic acid sequence,” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In one aspect, a nucleic acid molecule provided herein is an RNA molecule. In one aspect, a nucleic acid molecule provided herein is single-stranded. In one aspect, a nucleic acid molecule provided herein is double-stranded. A nucleic acid molecule can encode a polypeptide or a small RNA.

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell. As used herein, a “coding sequence” refers to a nucleic acid sequence that encodes a protein.

As used herein, the term “CpG site” or “CG site” refers to a region of DNA sequence where a cytosine and guanine is separated by only one phosphate group.

As used herein, the term “CpG island” of “CG island” refers to CpG sites that occur with a high frequency.

As used herein, the term “codon” refers to a sequence of three nucleotides.

As used herein, the term “codon optimized” refers to a code that is modified for enhanced expression in a host cell of interest by replacing at least one codon of a sequence with codons that are more frequently or most frequently used in the genes of the host cell while maintaining the original amino acid sequence.

As used herein, the term “enhancer” refers to a region of DNA sequence that operates to initiate, assist, affect, cause, and/or promote the transcription and expression of the associated transcribable DNA sequence or coding sequence, at least in certain tissue(s), developmental stage(s) and/or condition(s). In an aspect, an enhancer is a cis enhancer. In one aspect, an enhancer is a trans enhancer.

Enhancer sequences can be identified by utilizing genomic techniques well known in the art. Non-limiting examples include use of a reporter gene and next-generation sequencing methods such as chromatin immunoprecipitation sequencing (ChIP-seq), DNase I hypersensitivity sequencing (DNase-seq), micrococcal nuclease sequencing (MNase-seq), formaldehyde-assisted isolation of regulatory elements sequencing (FAIRE-seq), and assay for transposase accessible chromatin sequencing (ATAC-seq).

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s). As used herein, “regulatory elements” refer to any sequence elements that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence.

As used herein, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

As used herein, an “intron” refers to a nucleotide sequence that is removed by RNA splicing as a messenger RNA (mRNA) matures from a mRNA precursor.

As used herein, “mRNA” or “messenger RNA” refers to a single stranded RNA that corresponds to the genetic sequence of a gene.

Expression of mRNA can be measured using any suitable method known in the art. Non-limiting examples of measuring mRNA expression include quantitative reverse transcriptase polymerase chain reaction (qRT-PCR), RNA blot (e.g., a Northern blot), and RNA sequencing.

Differences in expression can be described as an absolute quantification or a relative quantification. See, for example, Livak and Schmittgen, Methods, 25:402-408 (2001).

As used herein, the term “glial” or “glial cell” refers to a non-neuronal cell in the CNS or the PNS. In an aspect, at least one glial cell is selected from the group consisting of at least one oligodendrocyte, at least one astrocyte, at least one NG2 cell, at least one ependymal cell, and at least one microglia. In one aspect, at least one glial cell is at least one oligodendrocyte. In one aspect, at least one glial cell is at least one NG2 cell. In one aspect, at least one glial cell is at least one ependymal cell. In one aspect, at least one glial cell is at least one microglia. In one aspect, at least one glial cell is at least one reactive astrocyte. In one aspect, at least one astrocyte is at least one reactive astrocyte.

As used herein, the term “astrocyte” refers to a glial cell that is an important component of the brain. An astrocyte is involved in supporting neuronal functions such as synapse formation and plasticity, potassium buffering, nutrient supply, the secretion and absorption of neural or glial transmitters, and maintenance of the blood-brain barrier. As used herein, the term “reactive astrocytes” refers to an abnormal status of astrocytes after injury or disease.

As used herein, the term “NG2 cell” or “polydendrocyte” refers to a glial cell that expresses chondroitin sulfate proteoglycan (CSPG4) and the alpha receptor for platelet-derived growth factor (PDGFRA).

As used herein, the term “neuron” or “neuronal cell” refers to an electrically excitable cell that communicates with other neurons via synapses. In an aspect, a neuron is selected from the group consisting of an unipolar neuron, a bipolar neuron, a pseudounipolar neuron, and a multipolar neuron. In one aspect, a neuron is an unipolar neuron. In one aspect, a neuron is a bipolar neuron. In one aspect, a neuron is apseudounipolar neuron. In one aspect, a neuron is a bipolar neuron. In one aspect, a neuron is selected from the group consisting of a sensory neuron, a motor neuron, and an interneuron. In one aspect, a neuron is a sensory neuron. In one aspect, a neuron is a motor neuron. In one aspect, a neuron is an interneuron.

As used herein, the term “functional neuron” refers to a neuron that can perform biological process. Without being limiting, examples of biological processes include processing and transmission of information and communication via chemical and electrical synapses.

As used herein, the term “glutamatergic neurons” refers to a subclass of neurons that produce glutamate and establish excitatory synapses. As used herein, the term “excitatory synapse” refers to a synapse in which an action potential in a presynaptic neuron increases the probability of an action potential occurring in a postsynaptic cell. As used herein, the term “action potential” or “nerve impulse” refers to an electrical impulse across the membrane of an axon. As used herein, the term “axon” or “nerve fiber” refers to a neuron that conducts action potentials. As used herein, the term “GABAergic neurons” refers to a subset of neurons that produce GABA and establish inhibitory synapses. As used herein, the term “GABA” or “gamma-Aminobutyric acid” refers to a compound that opens ion channels to allow the flow of negatively charged chloride ions into the cell or positively charged potassium ions out of the cell. As used herein, the term “inhibitory synapse” refers to a synapse that moves the membrane potential of a postsynaptic neuron away from the threshold for generating action potentials. As used herein, the term “dopaminergic neuron” refers to a subset of neurons that produce dopamine. As used herein, the term “dopamine” refers to a neurotransmitter. As used herein, the term “neurotransmitter” refers to endogenous chemicals that activate neurotransmissions. As used herein, the term “neurotransmission” refers to a process where neurotransmitters are released by the axon terminal of a neuron. As used herein, the term “acetyl cholinergic neuron” or “cholinergic neuron” refers to a subset of neurons that secrete acetylcholine. As used herein, the term “acetylcholine” refers to neurotransmitter. As used herein, the term “seratonergic neuron” refers to a subset of neurons that synthesizes serotonin. As used herein, the term “serotonin” refers to a neurotransmitter. As used herein, a “epinephrinergic neuron” refers to a neuron that releases epinephrine as the neurotransmitter. As used herein, the term “motor neuron” refers to a subset of neurons where the cell body is located in the motor cortex, brainstem, or the spinal cord and the axon projects to the spinal cord or outside the spinal cord and directly or indirectly controls muscles and glands. As used herein, the term peptidergic neuron refers to a subset of neurons that utilize small peptide molecules as their neurotransmitter.

In an aspect, a neuron is a functional neuron. In one aspect, a functional neuron is selected from the group consisting of glutamatergic neurons, GABAergic neurons, dopaminergic neurons, cholinergic neurons, seratonergic neurons, epinephrinergic neurons, motor neurons, and peptidergic neurons. In one aspect, a functional neuron is a glutamatergic neuron. In one aspect, a functional neuron is a GABAergic neuron. In one aspect, a functional neuron is a dopaminergic neuron. In one aspect, a functional neuron is a cholinergic neuron. In one aspect, a functional neuron is a seratonergic neuron. In one aspect, a functional neuron is an epinephrinergic neuron. In one aspect, a functional neuron is a motor neuron. In one aspect, a functional neuron is a peptidergic neuron.

As used herein, the term “converting” or “converted” refers to a cell type changing its physical morphology and/or biological function into a different physical morphology and/or different biological function. In an aspect, this disclosure provides the conversion of at least one glial cell into at least one neuron. In one aspect, conversion of at least one glial cell to at least one neuron occurs in the CNS or PNS. In one aspect, conversion of at least one glial cell to at least one neuron occurs in the CNS. In one aspect, conversion of at least one glial cell to at least one neuron occurs in the PNS.

In one aspect, this disclosure provides, and includes, an adeno-associated virus (AAV) vector comprising a human neurogenic differentiation 1 (hISL1) sequence comprising the nucleic acid sequence of SEQ ID NO: 6 and a human LIM-homeobox 3 (hLHX3) sequence comprising the nucleic acid sequence of SEQ ID NO: 13, where the hISL1 sequence and the hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 sequence and the hLHX3 sequence are operably linked to regulatory elements comprising: (a) glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In one aspect, this disclosure provides, and includes, an adeno-associated virus (AAV) vector comprising a nucleic acid coding sequence encoding a human neurogenic differentiation 1 (hISL1) protein comprising the amino acid coding sequence of SEQ ID NO: 10 and a nucleic acid coding sequence encoding a human LIM-homeobox 3 (hLHX3) protein comprising the amino acid sequence of SEQ ID NO: 14, where the hISL1 coding sequence and the hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 coding sequence and the hLHX3 coding sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising a neurogenic differentiation 1 (ISL1) nucleic acid coding sequence encoding a ISL1 protein and a LIM-homeobox 3 (LHX3) nucleic acid coding sequence encoding a LHX3 protein, where the ISL1 coding sequence and the LHX3 coding sequence are separated by a linker sequence, where the ISL1 coding sequence and the LHX3 coding sequence are operably linked to regulatory elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human neurogenic differentiation 1 (hISL1) sequence having a nucleic acid sequence of SEQ ID NO: 6 and a human LIM-homeobox 3 (hLHX3) sequence having a nucleic acid sequence of SEQ ID NO: 13, where the hISL1 sequence and the hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 sequence and hLHX3 sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated-virus (AAV) vector for converting glial cells to functional neurons in a human, where the AAV vector comprises a nucleic acid coding sequence encoding a human neurogenic differentiation 1 (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, where the hISL1 coding sequence and the hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 coding sequence and the hLHX3 coding sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated virus (AAV) vector for the treatment of a subject in need thereof, where the AAV vector comprises a neurogenic differentiation 1 (ISL1) sequence and a LIM-homeobox 3 (LHX3) sequence, where the ISL1 sequence and the LHX3 sequence are separated by a linker sequence, where the ISL1 sequence and LHX3 sequence are operably linked to expression control elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal.

In an aspect, this disclosure provides, and includes, an adeno-associated virus (AAV) vector comprising a human insulin gene enhancer protein (hISL1) sequence comprising the nucleic acid sequence of SEQ ID NO: 6, where the hISL1 sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising a nucleic acid coding sequence encoding a human insulin gene enhancer protein (hISL1) protein comprising the amino acid coding sequence of SEQ ID NO: 10, where the hISL1 coding sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising an insulin gene enhancer protein (ISL1) nucleic acid coding sequence encoding a ISL1 protein, where the ISL1 coding sequence is operably linked to regulatory elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human insulin gene enhancer protein (hISL1) sequence having a nucleic acid sequence of SEQ ID NO: 6, where the hISL1 sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated-virus (AAV) vector for converting glial cells to functional neurons in a human, where the AAV vector comprises a nucleic acid coding sequence encoding a human insulin gene enhancer protein (hISL1) protein comprising the amino acid sequence of SEQ ID NO: 10, where the hISL1 coding sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated virus (AAV) vector for the treatment of a subject in need thereof, where the AAV vector comprises an insulin gene enhancer protein (ISL1) sequence, where the ISL1 sequence is operably linked to expression control elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal.

In an aspect, this disclosure provides, and includes, an adeno-associated virus (AAV) vector comprising a human LIM-homeobox 3 (hLHX3) sequence comprising the nucleic acid sequence of SEQ ID NO: 13, where the hLHX3 sequence are operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising a nucleic acid coding sequence encoding a human LIM-homeobox 3 (hLHX3) protein comprising the amino acid sequence of SEQ ID NO: 14, where the hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where the hISL1 coding sequence and the hLHX3 coding sequence are operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, an adeno-associated virus (AAV) vector comprising a LIM-homeobox 3 (LHX3) nucleic acid coding sequence encoding a LHX3 protein, where the LHX3 coding sequence is operably linked to regulatory elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human LIM-homeobox 3 (hLHX3) sequence having a nucleic acid sequence of SEQ ID NO: 13, where the hLHX3 sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated-virus (AAV) vector for converting glial cells to functional neurons in a human, where the AAV vector comprises a nucleic acid coding sequence encoding a human LIM-homeobox 3 (hLHX3) protein comprising the amino acid sequence of SEQ ID NO: 14, where the hLHX3 coding sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a composition comprising an adeno-associated virus (AAV) vector for the treatment of a subject in need thereof, where the AAV vector comprises a LIM-homeobox 3 (LHX3) sequence, where the LHX3 sequence are operably linked to expression control elements comprising: (a) a glial fibrillary acidic protein (GFAP) promoter; (b) an enhancer; (c) a chimeric intron; (d) a woodchuck hepatitis virus posttranscriptional regulatory element (WPRE); and (e) a polyadenylation signal.

In an aspect, an AAV vector comprises a nucleic acid sequence encoding an AAV protein. In one aspect, an AAV vector comprises a nucleic acid sequence encoding a viral protein. Non-limiting examples of AAV proteins and viral proteins include rep and cap proteins.

Insulin gene enhancer protein (ISL1; also known as ISL LIM homeobox-1 and ISLET1) is a gene that encodes a transcription factor containing two N-terminal LIM domains and one C-terminal homeodomain. The encoded protein plays a role in the embryogenesis of pancreatic islets of Langerhans.

In an aspect, a ISL1 sequence is a human ISL1 (hISL1) sequence. In one aspect, a ISL1 sequence is selected from the group consisting of a chimpanzee ISL1 sequence, a bonobo ISL1 sequence, an orangutan ISL1 sequence, a gorilla ISL1 sequence, a macaque ISL1 sequence, a marmoset ISL1 sequence, a capuchin ISL1 sequence, a baboon ISL1 sequence, a gibbon ISL1 sequence, and a lemur ISL1 sequence. In one aspect, a ISL1 sequence is a chimpanzee ISL1 sequence. In one aspect, a ISL1 sequence is a bonobo ISL1 sequence. In one aspect, a ISL1 sequence is an orangutan ISL1 sequence. In one aspect, a ISL1 sequence is a gorilla ISL1 sequence. In one aspect, a ISL1 sequence is a macaque ISL1 sequence. In one aspect, a ISL1 sequence is a marmoset ISL1 sequence. In one aspect, a ISL1 sequence is a capuchin ISL1 sequence. In one aspect, a ISL1 sequence is a baboon ISL1 sequence. In one aspect, a ISL1 sequence is a gibbon ISL1 sequence. In one aspect, a ISL1 sequence is a lemur ISL1 sequence.

In an aspect, a ISL1 nucleic acid sequence comprises a sequence at least 70% identical to SEQ ID NO: 6, or the complement thereof. In one aspect, a ISL1 nucleic acid sequence comprises a sequence at least 75% identical to SEQ ID NO: 6, or the complement thereof. In one aspect, a ISL1 nucleic acid sequence comprises a sequence at least 80% identical to SEQ ID NO: 6, or the complement thereof. In one aspect, a ISL1 nucleic acid sequence comprises a sequence at least 85% identical to SEQ ID NO: 6, or the complement thereof. In one aspect, a ISL1 nucleic acid sequence comprises a sequence at least 90% identical to SEQ ID NO: 6, or the complement thereof. In one aspect, a ISL1 nucleic acid sequence comprises a sequence at least 91% identical to SEQ ID NO: 6, or the complement thereof. In one aspect, a ISL1 nucleic acid sequence comprises a sequence at least 92% identical to SEQ ID NO: 6, or the complement thereof. In one aspect, a ISL1 nucleic acid sequence comprises a sequence at least 93% identical to SEQ ID NO: 6, or the complement thereof. In one aspect, a ISL1 nucleic acid sequence comprises a sequence at least 94% identical to SEQ ID NO: 6, or the complement thereof. In one aspect, a ISL1 nucleic acid sequence comprises a sequence at least 95% identical to SEQ ID NO: 6, or the complement thereof. In one aspect, a ISL1 nucleic acid sequence comprises a sequence at least 96% identical to SEQ ID NO: 6, or the complement thereof. In one aspect, a ISL1 nucleic acid sequence comprises a sequence at least 97% identical to SEQ ID NO: 6, or the complement thereof. In one aspect, a ISL1 nucleic acid sequence comprises a sequence at least 98% identical to SEQ ID NO: 6, or the complement thereof. In one aspect, a ISL1 nucleic acid sequence comprises a sequence at least 99% identical to SEQ ID NO: 6, or the complement thereof. In one aspect, a ISL1 nucleic acid sequence comprises a sequence at least 99.5% identical to SEQ ID NO: 6, or the complement thereof. In one aspect, a ISL1 nucleic acid sequence comprises a sequence at least 99.8% identical to SEQ ID NO: 6, or the complement thereof. In one aspect, a ISL1 nucleic acid sequence comprises a sequence at least 99.9% identical to SEQ ID NO: 6, or the complement thereof. In one aspect, a ISL1 nucleic acid sequence comprises a sequence 100% identical to SEQ ID NO: 6, or the complement thereof.

In an aspect, a nucleic acid coding sequence encodes a ISL1 protein comprising an amino acid sequence at least 70% identical or similar to SEQ ID NO: 10. In one aspect, a nucleic acid coding sequence encodes a ISL1 protein comprising an amino acid sequence at least 75% identical or similar to SEQ ID NO: 10. In one aspect, a nucleic acid coding sequence encodes a ISL1 protein comprising an amino acid sequence at least 80% identical or similar to SEQ ID NO: 10. In one aspect, a nucleic acid coding sequence encodes a ISL1 protein comprising an amino acid sequence at least 85% identical or similar to SEQ ID NO: 10. In one aspect, a nucleic acid coding sequence encodes a ISL1 protein comprising an amino acid sequence at least 90% identical or similar to SEQ ID NO: 10. In one aspect, a nucleic acid coding sequence encodes a ISL1 protein comprising an amino acid sequence at least 91% identical or similar to SEQ ID NO: 10. In one aspect, a nucleic acid coding sequence encodes a ISL1 protein comprising an amino acid sequence at least 92% identical or similar to SEQ ID NO: 10. In one aspect, a nucleic acid coding sequence encodes a ISL1 protein comprising an amino acid sequence at least 93% identical or similar to SEQ ID NO: 10. In one aspect, a nucleic acid coding sequence encodes a ISL1 protein comprising an amino acid sequence at least 94% identical or similar to SEQ ID NO: 10. In one aspect, a nucleic acid coding sequence encodes a ISL1 protein comprising an amino acid sequence at least 95% identical or similar to SEQ ID NO: 10. In one aspect, a nucleic acid coding sequence encodes a ISL1 protein comprising an amino acid sequence at least 96% identical or similar to SEQ ID NO: 10. In one aspect, a nucleic acid coding sequence encodes a ISL1 protein comprising an amino acid sequence at least 97% identical or similar to SEQ ID NO: 10. In one aspect, a nucleic acid coding sequence encodes a ISL1 protein comprising an amino acid sequence at least 98% identical or similar to SEQ ID NO: 10. In one aspect, a nucleic acid coding sequence encodes a ISL1 protein comprising an amino acid sequence at least 99% identical or similar to SEQ ID NO: 10. In one aspect, a nucleic acid coding sequence encodes a ISL1 protein comprising an amino acid sequence at least 99.5% identical or similar to SEQ ID NO: 10. In one aspect, a nucleic acid coding sequence encodes a ISL1 protein comprising an amino acid sequence at least 99.8% identical or similar to SEQ ID NO: 10. In one aspect, a nucleic acid coding sequence encodes a ISL1 protein comprising an amino acid sequence at least 99.9% identical or similar to SEQ ID NO: 10. In one aspect, a nucleic acid coding sequence encodes a ISL1 protein comprising an amino acid sequence 100% identical or similar to SEQ ID NO: 10.

LIM-homeobox 3 (LHX3; also known as LIM3 and CPHD3) gene encodes for a protein from a family of proteins with a unique cysteine-rich zinc-binding domain (LIM domain).

In an aspect, a LHX3 sequence is a human LHX3 (hLHX3) sequence. In one aspect, a LHX3 sequence is selected from the group consisting of a chimpanzee LHX3 sequence, a bonobo LHX3 sequence, an orangutan LHX3 sequence, a gorilla LHX3 sequence, a macaque LHX3 sequence, a marmoset LHX3 sequence, a capuchin LHX3 sequence, a baboon LHX3 sequence, a gibbon LHX3 sequence, and a lemur LHX3 sequence. In one aspect, a LHX3 sequence is a chimpanzee LHX3 sequence. In one aspect, a LHX3 sequence is a bonobo LHX3 sequence. In one aspect, a LHX3 sequence is an orangutan LHX3 sequence. In one aspect, a LHX3 sequence is a gorilla LHX3 sequence. In one aspect, a LHX3 sequence is a macaque LHX3 sequence. In one aspect, a LHX3 sequence is a marmoset LHX3 sequence. In one aspect, a LHX3 sequence is a capuchin LHX3 sequence. In one aspect, a LHX3 sequence is a baboon LHX3 sequence. In one aspect, a LHX3 sequence is a gibbon LHX3 sequence. In one aspect, a LHX3 sequence is a lemur LHX3 sequence.

In an aspect, a LHX3 nucleic acid sequence comprises a sequence at least 70% identical to SEQ ID NO: 13, or the complement thereof. In one aspect, a LHX3 nucleic acid sequence comprises a sequence at least 75% identical to SEQ ID NO: 13, or the complement thereof. In one aspect, a LHX3 nucleic acid sequence comprises a sequence at least 80% identical to SEQ ID NO: 13, or the complement thereof. In one aspect, a LHX3 nucleic acid sequence comprises a sequence at least 85% identical to SEQ ID NO: 13, or the complement thereof. In one aspect, a LHX3 nucleic acid sequence comprises a sequence at least 90% identical to SEQ ID NO: 13, or the complement thereof. In one aspect, a LHX3 nucleic acid sequence comprises a sequence at least 91% identical to SEQ ID NO: 13, or the complement thereof. In one aspect, a LHX3 nucleic acid sequence comprises a sequence at least 92% identical to SEQ ID NO: 13, or the complement thereof. In one aspect, a LHX3 nucleic acid sequence comprises a sequence at least 93% identical to SEQ ID NO: 13, or the complement thereof. In one aspect, a LHX3 nucleic acid sequence comprises a sequence at least 94% identical to SEQ ID NO: 13, or the complement thereof. In one aspect, a LHX3 nucleic acid sequence comprises a sequence at least 95% identical to SEQ ID NO: 13, or the complement thereof. In one aspect, a LHX3 nucleic acid sequence comprises a sequence at least 913% identical to SEQ ID NO: 13, or the complement thereof. In one aspect, a LHX3 nucleic acid sequence comprises a sequence at least 97% identical to SEQ ID NO: 13, or the complement thereof. In one aspect, a LHX3 nucleic acid sequence comprises a sequence at least 98% identical to SEQ ID NO: 13, or the complement thereof. In one aspect, a LHX3 nucleic acid sequence comprises a sequence at least 99% identical to SEQ ID NO: 13, or the complement thereof. In one aspect, a LHX3 nucleic acid sequence comprises a sequence at least 99.5% identical to SEQ ID NO: 13, or the complement thereof. In one aspect, a LHX3 nucleic acid sequence comprises a sequence at least 99.8% identical to SEQ ID NO: 13, or the complement thereof. In one aspect, a LHX3 nucleic acid sequence comprises a sequence at least 99.9% identical to SEQ ID NO: 13, or the complement thereof. In one aspect, a LHX3 nucleic acid sequence comprises a sequence 100% identical to SEQ ID NO: 13, or the complement thereof.

In an aspect, a nucleic acid coding sequence encodes a LHX3 protein comprising an amino acid sequence at least 70% identical or similar to SEQ ID NO: 14. In one aspect, a nucleic acid coding sequence encodes a LHX3 protein comprising an amino acid sequence at least 75% identical or similar to SEQ ID NO: 14. In one aspect, a nucleic acid coding sequence encodes a LHX3 protein comprising an amino acid sequence at least 80% identical or similar to SEQ ID NO: 14. In one aspect, a nucleic acid coding sequence encodes a LHX3 protein comprising an amino acid sequence at least 85% identical or similar to SEQ ID NO: 14. In one aspect, a nucleic acid coding sequence encodes a LHX3 protein comprising an amino acid sequence at least 90% identical or similar to SEQ ID NO: 14. In one aspect, a nucleic acid coding sequence encodes a LHX3 protein comprising an amino acid sequence at least 91% identical or similar to SEQ ID NO: 14. In one aspect, a nucleic acid coding sequence encodes a LHX3 protein comprising an amino acid sequence at least 92% identical or similar to SEQ ID NO: 14. In one aspect, a nucleic acid coding sequence encodes a LHX3 protein comprising an amino acid sequence at least 93% identical or similar to SEQ ID NO: 14. In one aspect, a nucleic acid coding sequence encodes a LHX3 protein comprising an amino acid sequence at least 94% identical or similar to SEQ ID NO: 14. In one aspect, a nucleic acid coding sequence encodes a LHX3 protein comprising an amino acid sequence at least 95% identical or similar to SEQ ID NO: 14. In one aspect, a nucleic acid coding sequence encodes a LHX3 protein comprising an amino acid sequence at least 96% identical or similar to SEQ ID NO: 14. In one aspect, a nucleic acid coding sequence encodes a LHX3 protein comprising an amino acid sequence at least 97% identical or similar to SEQ ID NO: 14. In one aspect, a nucleic acid coding sequence encodes a LHX3 protein comprising an amino acid sequence at least 98% identical or similar to SEQ ID NO: 14. In one aspect, a nucleic acid coding sequence encodes a LHX3 protein comprising an amino acid sequence at least 99% identical or similar to SEQ ID NO: 14. In one aspect, a nucleic acid coding sequence encodes a LHX3 protein comprising an amino acid sequence at least 99.5% identical or similar to SEQ ID NO: 14. In one aspect, a nucleic acid coding sequence encodes a LHX3 protein comprising an amino acid sequence at least 99.8% identical or similar to SEQ ID NO: 14. In one aspect, a nucleic acid coding sequence encodes a LHX3 protein comprising an amino acid sequence at least 99.9% identical or similar to SEQ ID NO: 14. In one aspect, a nucleic acid coding sequence encodes a LHX3 protein comprising an amino acid sequence 140% identical or similar to SEQ ID NO: 14.

In an aspect, an AAV vector comprises a ISL1 sequence and a LHX3 sequence. In one aspect, an AAV vector comprises a ISL1 sequence. In one aspect, an AAV comprises a LHX3 sequence.

As used herein, “linkers” or “spacers” are short sequences that separate multiple protein and coding domains. Linkers can be cleavable or non-cleavable and facilitate multigene co-expression in single vectors.

As used herein, “2A self-cleaving peptides” or “2A peptides” are a class of linkers that can induce the cleaving of recombinant protein in a cell.

As used herein, “P2A linker” refers to the porcine teschovirus-1 (P2A) linker, which is a member of the 2A self-cleaving peptides.

In one aspect, a P2A linker has a nucleic acid sequence selected from the group consisting of SEQ ID NOs: 15 and 18. In one aspect, a P2A linker has a nucleic acid sequence of SEQ ID NO: 15. In one aspect, a P2A linker has a nucleic acid sequence of SEQ ID NO: 18. In one aspect, a P2A linker protein has a nucleic acid coding sequence of SEQ ID NO: 20.

As used herein, “T2A linker” refers to thosea asigna virus 2A (T2A) linker, which is a member of the 2A self-cleaving peptides.

In one aspect, a T2A linker has a nucleic acid sequence selected from the group consisting of SEQ ID NOs: 16 and 19. In one aspect, a T2A linker has a nucleic acid sequence of SEQ ID NO: 16. In one aspect, a T2A linker has a nucleic acid sequence of SEQ ID NO: 19. In one aspect, a T2A linker protein has a nucleic acid coding sequence of SEQ ID NO: 21.

As used herein, “E2A linker” refers to equine rhinitis A virus (E2A) linker, which is a member of the 2A self-cleaving peptides.

As used herein, “F2A linker” refers to foot and mouse disease virus (F2A) linker, which is a member of the 2A self-cleaving peptides.

In an aspect, a linker is selected from the group consisting of a P2A linker, a T2A linker, a E2A linker, and a F2A linker. In one aspect, a linker is a P2A linker. In one aspect, a linker is a T2A linker. In one aspect, a linker is a E2A linker. In one aspect, a linker is a F2A linker.

In an aspect, a linker sequence comprises a P2A linker. In one aspect, a P2A linker nucleic acid sequence comprises a sequence at least 70% identical to SEQ ID NO: 15, or the complement thereof. In one aspect, a P2A linker nucleic acid sequence comprises a sequence at least 75% identical to SEQ ID NO: 15, or the complement thereof. In one aspect, a P2A linker nucleic acid sequence comprises a sequence at least 80% identical to SEQ ID NO: 15, or the complement thereof. In one aspect, a P2A linker nucleic acid sequence comprises a sequence at least 85% identical to SEQ ID NO: 15, or the complement thereof. In one aspect, a P2A linker nucleic acid sequence comprises a sequence at least 90% identical to SEQ ID NO: 15, or the complement thereof. In one aspect, a P2A linker nucleic acid sequence comprises a sequence at least 91% identical to SEQ ID NO: 15, or the complement thereof. In one aspect, a P2A linker nucleic acid sequence comprises a sequence at least 92% identical to SEQ ID NO: 15, or the complement thereof. In one aspect, a P2A linker nucleic acid sequence comprises a sequence at least 93% identical to SEQ ID NO: 15, or the complement thereof. In one aspect, a P2A linker nucleic acid sequence comprises a sequence at least 94% identical to SEQ ID NO: 15, or the complement thereof. In one aspect, a P2A linker nucleic acid sequence comprises a sequence at least 95% identical to SEQ ID NO: 15, or the complement thereof. In one aspect, a P2A linker nucleic acid sequence comprises a sequence at least 96% identical to SEQ ID NO: 15, or the complement thereof. In one aspect, a P2A linker nucleic acid sequence comprises a sequence at least 97% identical to SEQ ID NO: 15, or the complement thereof. In one aspect, a P2A linker nucleic acid sequence comprises a sequence at least 98% identical to SEQ ID NO: 15, or the complement thereof. In one aspect, a P2A linker nucleic acid sequence comprises a sequence at least 99% identical to SEQ ID NO: 15, or the complement thereof. In one aspect, a P2A linker nucleic acid sequence comprises a sequence at least 99.5% identical to SEQ ID NO: 15, or the complement thereof. In one aspect, a P2A linker nucleic acid sequence comprises a sequence at least 99.8% identical to SEQ ID NO: 15, or the complement thereof. In one aspect, a P2A linker nucleic acid sequence comprises a sequence at least 99.9% identical to SEQ ID NO: 15, or the complement thereof. In one aspect, a P2A linker nucleic acid sequence comprises a sequence 100% identical to SEQ ID NO: 15, or the complement thereof.

In an aspect, a linker sequence comprises a P2A linker. In one aspect, a P2A linker nucleic acid sequence comprises a sequence at least 70% identical to SEQ ID NO: 18, or the complement thereof. In one aspect, a P2A linker nucleic acid sequence comprises a sequence at least 75% identical to SEQ ID NO: 18, or the complement thereof. In one aspect, a P2A linker nucleic acid sequence comprises a sequence at least 80% identical to SEQ ID NO: 18, or the complement thereof. In one aspect, a P2A linker nucleic acid sequence comprises a sequence at least 85% identical to SEQ ID NO: 18, or the complement thereof. In one aspect, a P2A linker nucleic acid sequence comprises a sequence at least 90% identical to SEQ ID NO: 18, or the complement thereof. In one aspect, a P2A linker nucleic acid sequence comprises a sequence at least 91% identical to SEQ ID NO: 18, or the complement thereof. In one aspect, a P2A linker nucleic acid sequence comprises a sequence at least 92% identical to SEQ ID NO: 18, or the complement thereof. In one aspect, a P2A linker nucleic acid sequence comprises a sequence at least 93% identical to SEQ ID NO: 18, or the complement thereof. In one aspect, a P2A linker nucleic acid sequence comprises a sequence at least 94% identical to SEQ ID NO: 18, or the complement thereof. In one aspect, a P2A linker nucleic acid sequence comprises a sequence at least 95% identical to SEQ ID NO: 18, or the complement thereof. In one aspect, a P2A linker nucleic acid sequence comprises a sequence at least 96% identical to SEQ ID NO: 18, or the complement thereof. In one aspect, a P2A linker nucleic acid sequence comprises a sequence at least 97% identical to SEQ ID NO: 18, or the complement thereof. In one aspect, a P2A linker nucleic acid sequence comprises a sequence at least 98% identical to SEQ ID NO: 18, or the complement thereof. In one aspect, a P2A linker nucleic acid sequence comprises a sequence at least 99% identical to SEQ ID NO: 18, or the complement thereof. In one aspect, a P2A linker nucleic acid sequence comprises a sequence at least 99.5% identical to SEQ ID NO: 18, or the complement thereof. In one aspect, a P2A linker nucleic acid sequence comprises a sequence at least 99.8% identical to SEQ ID NO: 18, or the complement thereof. In one aspect, a P2A linker nucleic acid sequence comprises a sequence at least 99.9% identical to SEQ ID NO: 18, or the complement thereof. In one aspect, a P2A linker nucleic acid sequence comprises a sequence 100% identical to SEQ ID NO: 18, or the complement thereof.

In an aspect, a nucleic acid coding sequence encodes a P2A protein comprising an amino acid sequence at least 70% identical or similar to SEQ ID NO: 20. In one aspect, a nucleic acid coding sequence encodes a P2A protein comprising an amino acid sequence at least 75% identical or similar to SEQ ID NO: 20. In one aspect, a nucleic acid coding sequence encodes a P2A protein comprising an amino acid sequence at least 80% identical or similar to SEQ ID NO: 20. In one aspect, a nucleic acid coding sequence encodes a P2A protein comprising an amino acid sequence at least 85% identical or similar to SEQ ID NO: 20. In one aspect, a nucleic acid coding sequence encodes a P2A protein comprising an amino acid sequence at least 90% identical or similar to SEQ ID NO: 20. In one aspect, a nucleic acid coding sequence encodes a P2A protein comprising an amino acid sequence at least 91% identical or similar to SEQ ID NO: 20. In one aspect, a nucleic acid coding sequence encodes a P2A protein comprising an amino acid sequence at least 92% identical or similar to SEQ ID NO: 20. In one aspect, a nucleic acid coding sequence encodes a P2A protein comprising an amino acid sequence at least 93% identical or similar to SEQ ID NO: 20. In one aspect, a nucleic acid coding sequence encodes a P2A protein comprising an amino acid sequence at least 94% identical or similar to SEQ ID NO: 20. In one aspect, a nucleic acid coding sequence encodes a P2A protein comprising an amino acid sequence at least 95% identical or similar to SEQ ID NO: 20. In one aspect, a nucleic acid coding sequence encodes a P2A protein comprising an amino acid sequence at least 96% identical or similar to SEQ ID NO: 20. In one aspect, a nucleic acid coding sequence encodes a P2A protein comprising an amino acid sequence at least 97% identical or similar to SEQ ID NO: 20. In one aspect, a nucleic acid coding sequence encodes a P2A protein comprising an amino acid sequence at least 98% identical or similar to SEQ ID NO: 20. In one aspect, a nucleic acid coding sequence encodes a P2A protein comprising an amino acid sequence at least 99% identical or similar to SEQ ID NO: 20. In one aspect, a nucleic acid coding sequence encodes a P2A protein comprising an amino acid sequence at least 99.5% identical or similar to SEQ ID NO: 20. In one aspect, a nucleic acid coding sequence encodes a P2A protein comprising an amino acid sequence at least 99.8% identical or similar to SEQ ID NO: 20. In one aspect, a nucleic acid coding sequence encodes a P2A protein comprising an amino acid sequence at least 99.9% identical or similar to SEQ ID NO: 20. In one aspect, a nucleic acid coding sequence encodes a P2A protein comprising an amino acid sequence 100% identical or similar to SEQ ID NO: 20.

In an aspect, a linker sequence comprises a T2A linker. In one aspect, a T2A linker nucleic acid sequence comprises a sequence at least 70% identical to SEQ ID NO: 16, or the complement thereof. In one aspect, a T2A linker nucleic acid sequence comprises a sequence at least 75% identical to SEQ ID NO: 16, or the complement thereof. In one aspect, a T2A linker nucleic acid sequence comprises a sequence at least 80% identical to SEQ ID NO: 16, or the complement thereof. In one aspect, a T2A linker nucleic acid sequence comprises a sequence at least 85% identical to SEQ ID NO: 16, or the complement thereof. In one aspect, a T2A linker nucleic acid sequence comprises a sequence at least 90% identical to SEQ ID NO: 16, or the complement thereof. In one aspect, a T2A linker nucleic acid sequence comprises a sequence at least 91% identical to SEQ ID NO: 16, or the complement thereof. In one aspect, a T2A linker nucleic acid sequence comprises a sequence at least 92% identical to SEQ ID NO: 16, or the complement thereof. In one aspect, a T2A linker nucleic acid sequence comprises a sequence at least 93% identical to SEQ ID NO: 16, or the complement thereof. In one aspect, a T2A linker nucleic acid sequence comprises a sequence at least 94% identical to SEQ ID NO: 16, or the complement thereof. In one aspect, a T2A linker nucleic acid sequence comprises a sequence at least 95% identical to SEQ ID NO: 16, or the complement thereof. In one aspect, a T2A linker nucleic acid sequence comprises a sequence at least 917% identical to SEQ ID NO: 16, or the complement thereof. In one aspect, a T2A linker nucleic acid sequence comprises a sequence at least 97% identical to SEQ ID NO: 16, or the complement thereof. In one aspect, a T2A linker nucleic acid sequence comprises a sequence at least 98% identical to SEQ ID NO: 16, or the complement thereof. In one aspect, a T2A linker nucleic acid sequence comprises a sequence at least 99% identical to SEQ ID NO: 16, or the complement thereof. In one aspect, a T2A linker nucleic acid sequence comprises a sequence at least 99.5% identical to SEQ ID NO: 16, or the complement thereof. In one aspect, a T2A linker nucleic acid sequence comprises a sequence at least 99.8% identical to SEQ ID NO: 16, or the complement thereof. In one aspect, a T2A linker nucleic acid sequence comprises a sequence at least 99.9% identical to SEQ ID NO: 16, or the complement thereof. In one aspect, a T2A linker nucleic acid sequence comprises a sequence 100% identical to SEQ ID NO: 16, or the complement thereof.

In an aspect, a linker sequence comprises a T2A linker. In one aspect, a T2A linker nucleic acid sequence comprises a sequence at least 70% identical to SEQ ID NO: 19, or the complement thereof. In one aspect, a T2A linker nucleic acid sequence comprises a sequence at least 75% identical to SEQ ID NO: 19, or the complement thereof. In one aspect, a T2A linker nucleic acid sequence comprises a sequence at least 80% identical to SEQ ID NO: 19, or the complement thereof. In one aspect, a T2A linker nucleic acid sequence comprises a sequence at least 85% identical to SEQ ID NO: 19, or the complement thereof. In one aspect, a T2A linker nucleic acid sequence comprises a sequence at least 90% identical to SEQ ID NO: 19, or the complement thereof. In one aspect, a T2A linker nucleic acid sequence comprises a sequence at least 91% identical to SEQ ID NO: 19, or the complement thereof. In one aspect, a T2A linker nucleic acid sequence comprises a sequence at least 92% identical to SEQ ID NO: 19, or the complement thereof. In one aspect, a T2A linker nucleic acid sequence comprises a sequence at least 93% identical to SEQ ID NO: 19, or the complement thereof. In one aspect, a T2A linker nucleic acid sequence comprises a sequence at least 94% identical to SEQ ID NO: 19, or the complement thereof. In one aspect, a T2A linker nucleic acid sequence comprises a sequence at least 95% identical to SEQ ID NO: 19, or the complement thereof. In one aspect, a T2A linker nucleic acid sequence comprises a sequence at least 96% identical to SEQ ID NO: 19, or the complement thereof. In one aspect, a T2A linker nucleic acid sequence comprises a sequence at least 97% identical to SEQ ID NO: 19, or the complement thereof. In one aspect, a T2A linker nucleic acid sequence comprises a sequence at least 98% identical to SEQ ID NO: 19, or the complement thereof. In one aspect, a T2A linker nucleic acid sequence comprises a sequence at least 99% identical to SEQ ID NO: 19, or the complement thereof. In one aspect, a T2A linker nucleic acid sequence comprises a sequence at least 99.5% identical to SEQ ID NO: 19, or the complement thereof. In one aspect, a T2A linker nucleic acid sequence comprises a sequence at least 99.8% identical to SEQ ID NO: 19, or the complement thereof. In one aspect, a T2A linker nucleic acid sequence comprises a sequence at least 99.9% identical to SEQ ID NO: 19, or the complement thereof. In one aspect, a T2A linker nucleic acid sequence comprises a sequence 100% identical to SEQ ID NO: 19, or the complement thereof.

In an aspect, a nucleic acid coding sequence encodes a T2A protein comprising an amino acid sequence at least 70% identical or similar to SEQ ID NO: 21. In one aspect, a nucleic acid coding sequence encodes a T2A protein comprising an amino acid sequence at least 75% identical or similar to SEQ ID NO: 21. In one aspect, a nucleic acid coding sequence encodes a T2A protein comprising an amino acid sequence at least 80% identical or similar to SEQ ID NO: 21. In one aspect, a nucleic acid coding sequence encodes a T2A protein comprising an amino acid sequence at least 85% identical or similar to SEQ ID NO: 21. In one aspect, a nucleic acid coding sequence encodes a T2A protein comprising an amino acid sequence at least 90% identical or similar to SEQ ID NO: 21. In one aspect, a nucleic acid coding sequence encodes a T2A protein comprising an amino acid sequence at least 91% identical or similar to SEQ ID NO: 21. In one aspect, a nucleic acid coding sequence encodes a T2A protein comprising an amino acid sequence at least 92% identical or similar to SEQ ID NO: 21. In one aspect, a nucleic acid coding sequence encodes a T2A protein comprising an amino acid sequence at least 93% identical or similar to SEQ ID NO: 21. In one aspect, a nucleic acid coding sequence encodes a T2A protein comprising an amino acid sequence at least 94% identical or similar to SEQ ID NO: 21. In one aspect, a nucleic acid coding sequence encodes a T2A protein comprising an amino acid sequence at least 95% identical or similar to SEQ ID NO: 21. In one aspect, a nucleic acid coding sequence encodes a T2A protein comprising an amino acid sequence at least 96% identical or similar to SEQ ID NO: 21. In one aspect, a nucleic acid coding sequence encodes a T2A protein comprising an amino acid sequence at least 97% identical or similar to SEQ ID NO: 21. In one aspect, a nucleic acid coding sequence encodes a T2A protein comprising an amino acid sequence at least 98% identical or similar to SEQ ID NO: 21. In one aspect, a nucleic acid coding sequence encodes a T2A protein comprising an amino acid sequence at least 99% identical or similar to SEQ ID NO: 21. In one aspect, a nucleic acid coding sequence encodes a T2A protein comprising an amino acid sequence at least 99.5% identical or similar to SEQ ID NO: 21. In one aspect, a nucleic acid coding sequence encodes a T2A protein comprising an amino acid sequence at least 99.8% identical or similar to SEQ ID NO: 21. In one aspect, a nucleic acid coding sequence encodes a T2A protein comprising an amino acid sequence at least 99.9% identical or similar to SEQ ID NO: 21. In one aspect, a nucleic acid coding sequence encodes a T2A protein comprising an amino acid sequence 100% identical or similar to SEQ ID NO: 21.

Glial fibrillary acid protein (GFAP); also referred to as glial fibrillary acidic protein is a member of the type III intermediate filament family of proteins that is expressed in the central nervous system and plays a role in cell communication and the functioning of the blood-brain barrier.

In an aspect, the promoter is selected from the group consisting of GFAP promoter, Sox9 promoter, S100b promoter, Aldh1l1 promoter, Lipocalin 2 (Lcn2) promoter, glutamine synthetase promoter, Aquaporin-4 (AQP4) promoter, oligodendrocyte transcription factor (Olig2) promoter, and synapsin promoter, NG2 promoter, ionized calcium binding adaptor molecule 1 (Iba1) promoter, cluster of differentiation 86 (CD86) promoter, platelet-derived growth factor receptor alpha (PDGFRA) promoter, platelet-derived growth factor receptor beta (PDGFRB) promoter, elongation factor 1-alpha (EF1a) promoter, CAG promoter, cytomegalovirus (CMV) promoter, ubiquitin promoter. In one aspect, the promoter is GFAP promoter. In one aspect, the promoter is Sox9 promoter. In one aspect, the promoter is S100b promoter. In one aspect, the promoter is Aldh1l1 promoter. In one aspect, the promoter is Lcn2 promoter. In one aspect, the promoter is glutamine synthetase promoter. In one aspect, the promoter is AQP4 promoter. In one aspect, the promoter is Olig2 promoter. In one aspect, the promoter is synapsin promoter. In one aspect, the promoter is Iba1 promoter. In one aspect, the promoter is CD86 promoter. In one aspect, the promoter is PDGFRA promoter. In one aspect, the promoter is PDGFRB promoter. In one aspect, the promoter is EF1a promoter. In one aspect, the promoter is CAG promoter. In one aspect, the promoter is CMV promoter. In one aspect, the promoter is ubiquitin promoter.

In an aspect, a GFAP promoter is a promoter directing astrocyte-specific expression of a protein called glial fibrillary acidic protein (GFAP) in cells. In one aspect, a GFAP promoter sequence is a human GFAP (hGFAP) promoter sequence. In one aspect, a GFAP promoter is selected from the group consisting of GfaABC1D, Gfa1.6, and hGFA2.2. In one aspect, a GFAP promoter is GfaABC1D. In one aspect, a GFAP promoter is Gfa1.6. In one aspect, a GFAP promoter is hGFA2.2. In one aspect, GFAP GfaABC1D is SEQ ID NO: 3. In one aspect, GFAP Gfa1.6 is SEQ ID NO: 4. In one aspect, hGFa2.2 is SEQ ID NO: 12. In one aspect, a GFAP promoter is selected from the group consisting of SEQ ID NOs: 3, 4, and 12. In one aspect, a GFAP promoter is SEQ ID NO: 3. In one aspect, a GFAP promoter is SEQ ID NO: 4. In one aspect, a GFAP promoter is SEQ ID NO: 12.

In one aspect, a GFAP promoter sequence is selected from the group consisting of a chimpanzee GFAP promoter sequence, a bonobo GFAP promoter sequence, an orangutan GFAP promoter sequence, a gorilla GFAP promoter sequence, a macaque GFAP promoter sequence, a marmoset GFAP promoter sequence, a capuchin GFAP promoter sequence, a baboon GFAP promoter sequence, a gibbon GFAP promoter sequence, and a lemur GFAP promoter sequence.

In one aspect, a GFAP promoter sequence is a chimpanzee GFAP promoter sequence. In one aspect, a GFAP promoter sequence is a bonobo GFAP promoter sequence. In one aspect, a GFAP promoter sequence is an orangutan GFAP promoter sequence. In one aspect, a GFAP promoter sequence is a gorilla GFAP promoter sequence. In one aspect, a GFAP promoter sequence is a macaque GFAP promoter sequence. In one aspect, a GFAP promoter sequence is a marmoset GFAP promoter sequence. In one aspect, a GFAP promoter sequence is a capuchin GFAP promoter sequence. In one aspect, a GFAP promoter sequence is a baboon GFAP promoter sequence. In one aspect, a GFAP promoter sequence is a gibbon GFAP promoter sequence. In one aspect, a GFAP promoter sequence is a lemur GFAP promoter sequence.

In an aspect, a GFAP promoter sequence comprises at least 100 nucleotides. In one aspect, a GFAP promoter comprises at least 500 nucleotides. In a further aspect, a GFAP promoter comprises at least 1000 nucleotides. In still another aspect, a GFAP promoter comprises at least 1500 nucleotides.

It is appreciated in the art that a fragment of a promoter sequence can function to drive transcription of an operably linked nucleic acid molecule. For example, without being limiting, if a 1000 nucleotides promoter is truncated to 500 nucleotides, and the 500 nucleotides fragment is capable of driving transcription, the 500 nucleotides fragment is referred to as a “functional fragment.”

In an aspect, a promoter comprises at least 10 nucleotides. In one aspect, a promoter comprises at least 50 nucleotides. In one aspect, a promoter comprises at least 100 nucleotides. In one aspect, an intron comprises at least 150 nucleotides. In one aspect, a promoter comprises at least 200 nucleotides. In one aspect, a promoter comprises at least 250 nucleotides. In one aspect, a promoter comprises at least 300 nucleotides. In one aspect, a promoter comprises at least 350 nucleotides. In one aspect, a promoter comprises at least 400 nucleotides. In one aspect, a promoter comprises at least 450 nucleotides. In one aspect, a promoter comprises at least 500 nucleotides. In one aspect, a promoter comprises between 50 nucleotides and 7500 nucleotides. In one aspect, a promoter comprises between 50 nucleotides and 5000 nucleotides. In one aspect, a promoter comprises between 50 nucleotides and 2500 nucleotides. In one aspect, a promoter comprises between 50 nucleotides and 1000 nucleotides. In one aspect, a promoter comprises between 50 nucleotides and 500 nucleotides. In one aspect, a promoter comprises between 10 nucleotides and 7500 nucleotides. In one aspect, a promoter comprises between 10 nucleotides and 5000 nucleotides. In one aspect, a promoter comprises between 10 nucleotides and 2500 nucleotides. In one aspect, a promoter comprises between 10 nucleotides and 1000 nucleotides. In one aspect, a promoter comprises between 10 nucleotides and 500 nucleotides

In an aspect, a GFAP promoter nucleic acid sequence comprises a sequence at least 70% identical to a nucleic acid sequence selected from the group consisting of SEQ ID NOs: 3, 4, and 12, or functional fragment thereof. In one aspect, a GFAP promoter nucleic acid sequence comprises a sequence at least 75% identical to a nucleic acid sequence selected from the group consisting of SEQ ID NOs: 3, 4, and 12, or functional fragment thereof. In one aspect, a GFAP promoter nucleic acid sequence comprises a sequence at least 80% identical to a nucleic acid sequence selected from the group consisting of SEQ ID NOs: 3, 4, and 12, or functional fragment thereof. In one aspect, a GFAP promoter nucleic acid sequence comprises a sequence at least 85% identical to a nucleic acid sequence selected from the group consisting of SEQ ID NOs: 3, 4, and 12, or functional fragment thereof. In one aspect, a GFAP promoter nucleic acid sequence comprises a sequence at least 90% identical to a nucleic acid sequence selected from the group consisting of SEQ ID NOs: 3, 4, and 12, or functional fragment thereof. In one aspect, a GFAP promoter nucleic acid sequence comprises a sequence at least 91% identical to a nucleic acid sequence selected from the group consisting of SEQ ID NOs: 3, 4, and 12, or functional fragment thereof. In one aspect, a GFAP promoter nucleic acid sequence comprises a sequence at least 92% identical to a nucleic acid sequence selected from the group consisting of SEQ ID NOs: 3, 4, and 12, or functional fragment thereof. In one aspect, a GFAP promoter nucleic acid sequence comprises a sequence at least 93% identical to a nucleic acid sequence selected from the group consisting of SEQ ID NOs: 3, 4, and 12, or functional fragment thereof. In one aspect, a GFAP promoter nucleic acid sequence comprises a sequence at least 94% identical to a nucleic acid sequence selected from the group consisting of SEQ ID NOs: 3, 4, and 12, or functional fragment thereof. In one aspect, a GFAP promoter nucleic acid sequence comprises a sequence at least 95% identical to a nucleic acid sequence selected from the group consisting of SEQ ID NOs: 3, 4, and 12, or functional fragment thereof. In one aspect, a GFAP promoter nucleic acid sequence comprises a sequence at least 96% identical to a nucleic acid sequence selected from the group consisting of SEQ ID NOs: 3, 4, and 12, or functional fragment thereof. In one aspect, a GFAP promoter nucleic acid sequence comprises a sequence at least 97% identical to a nucleic acid sequence selected from the group consisting of SEQ ID NOs: 3, 4, and 12, or functional fragment thereof. In one aspect, a GFAP promoter nucleic acid sequence comprises a sequence at least 98% identical to a nucleic acid sequence selected from the group consisting of SEQ ID NOs: 3, 4, and 12, or functional fragment thereof. In one aspect, a GFAP promoter nucleic acid sequence comprises a sequence at least 99% identical to a nucleic acid sequence selected from the group consisting of SEQ ID NOs: 3, 4, and 12, or functional fragment thereof. In one aspect, a GFAP promoter nucleic acid sequence comprises a sequence at least 99.5% identical to a nucleic acid sequence selected from the group consisting of SEQ ID NOs: 3, 4, and 12, or functional fragment thereof. In one aspect, a GFAP promoter nucleic acid sequence comprises a sequence at least 99.8% identical to a nucleic acid sequence selected from the group consisting of SEQ ID NOs: 3, 4, and 12, or functional fragment thereof. In one aspect, a GFAP promoter nucleic acid sequence comprises a sequence at least 99.9% identical to a nucleic acid sequence selected from the group consisting of SEQ ID NOs: 3, 4, and 12, or functional fragment thereof. In one aspect, a GFAP promoter nucleic acid sequence comprises a sequence 100% identical to a nucleic acid sequence selected from the group consisting of SEQ ID NOs: 3, 4, and 12, or functional fragment thereof.

As used herein, the term “brain” refers to an organ that functions as the center of the nervous system. In an aspect, a brain comprises a cerebrum, a cerebral cortex, a cerebellum, and/or a brain stem.

As used herein, the term “cerebral cortex” refers to the outer layer of neural tissue of the cerebrum.

As used herein, the term “striatum” or “corpus striatum” refers to a cluster of neurons in the subcortical basal ganglia of the forebrain and comprises the ventral striatum and dorsal striatum.

As used herein, the term “substantia nigra” refers to a cluster of neurons in the subcortical basal ganglia of the midbrain and comprises the pars compacta and the pars reticulata.

As used herein, the term “forebrain” refers to the forward-most portion of the brain.

As used herein, the term “putamen” refers to a round structure at the base of the forebrain and is a component of the dorsal striatum.

As used herein, the term “caudate nucleus” refers to a structure at the base of the forebrain and is a component of the dorsal striatum.

As used herein, the term “subcortical basal ganglia” refers to a cluster of neurons in the deep cerebral hemispheres of the brain.

As used herein, the term “spinal cord” refers to a structure that functions in the transmission of nerve signals from the motor cortex to the body.

As used herein, the term “motor cortex” refers to a region in the frontal lobe of the cerebral cortex that is involved in the planning, control, and execution of voluntary movements.

In an aspect, a method provided herein converts reactive astrocytes to functional neurons in the brain. In one aspect, a method provided herein converts reactive astrocytes to functional neurons in a cerebral cortex of the brain. In one aspect, a method provided herein coverts reactive astrocytes to functional neurons in a striatum of the brain. In one aspect, a method provided herein converts reactive astrocytes to functional neurons in a dorsal striatum of the brain.

In one aspect, a method provided herein converts reactive astrocytes to functional neurons in a spinal cord of the brain. In one aspect, a method provided herein converts reactive astrocytes to functional neurons in a putamen of the brain. In one aspect, a method provided herein converts reactive astrocytes to functional neurons in a caudate nucleus of the brain. In one aspect, a method provided herein converts reactive astrocytes to functional neurons in a substantia nigra of the brain.

Elongation factor-1 alpha (EF-1 alpha; also referred to as eEF1a1) is an isoform of the alpha subunit of the elongation factor 1 complex. The complex is involved in the enzymatic delivery of aminoacyl tRNAs to the ribosome. The EF-1 alpha isoform is expressed in the brain, placenta, lung, liver, kidney, and pancreas.

In an aspect, an enhancer sequence from the EF-1 alpha promoter is a human enhancer sequence from the EF-1 alpha promoter. In one aspect, an enhancer sequence from the EF-1 alpha promoter is selected form the group consisting of a chimpanzee enhancer sequence from the EF-1 alpha promoter, a bonobo enhancer sequence from the EF-1 alpha promoter, an orangutan enhancer sequence from the EF-1 alpha promoter, a gorilla enhancer sequence from the EF-1 alpha promoter, a macaque enhancer sequence from the EF-1 alpha promoter, a marmoset enhancer sequence from the EF-1 alpha promoter, a capuchin enhancer sequence from the EF-1 alpha promoter, a baboon enhancer sequence from the EF-1 alpha promoter, a gibbon enhancer sequence from the EF-1 alpha promoter, and a lemur enhancer sequence from the EF-1 alpha promoter. In one aspect, an enhancer sequence from the EF-1 alpha promoter is a chimpanzee an enhancer sequence from the EF-1 alpha promoter. In one aspect, an enhancer sequence from the EF-1 alpha promoter is a bonobo enhancer sequence from the EF-1 alpha promoter. In one aspect, an enhancer sequence from the EF-1 alpha promoter is an orangutan enhancer sequence from the EF-1 alpha promoter. In one aspect, an enhancer sequence from the EF-1 alpha promoter is a gorilla enhancer sequence from the EF-1 alpha promoter. In one aspect, an enhancer sequence from the EF-1 alpha promoter is a macaque enhancer sequence from the EF-1 alpha promoter. In one aspect, enhancer sequence from the EF-1 alpha promoter is a marmoset enhancer sequence from the EF-1 alpha promoter. In one aspect, enhancer sequence from the EF-1 alpha promoter is a capuchin enhancer sequence from the EF-1 alpha promoter. In one aspect, enhancer sequence from the EF-1 alpha promoter is a baboon enhancer sequence from the EF-1 alpha promoter. In one aspect, enhancer sequence from the EF-1 alpha promoter is a gibbon enhancer sequence from the EF-1 alpha promoter. In one aspect, enhancer sequence from the EF-1 alpha promoter is a lemur enhancer sequence from the EF-1 alpha promoter.

In an aspect, an enhancer from the EF-1 alpha promoter nucleic acid sequence comprises a sequence at least 70% identical to SEQ ID NO: 2, or the complement thereof. In one aspect, an enhancer from the EF-1 alpha promoter nucleic acid sequence comprises a sequence at least 75% identical to SEQ ID NO: 2, or the complement thereof. In one aspect, an enhancer from the EF-1 alpha promoter nucleic acid sequence comprises a sequence at least 80% identical to SEQ ID NO: 2, or the complement thereof. In one aspect, an enhancer from the EF-1 alpha promoter nucleic acid sequence comprises a sequence at least 85% identical to SEQ ID NO: 2, or the complement thereof. In one aspect, an enhancer from the EF-1 alpha promoter nucleic acid sequence comprises a sequence at least 90% identical to SEQ ID NO: 2, or the complement thereof. In one aspect, an enhancer from the EF-1 alpha promoter nucleic acid sequence comprises a sequence at least 91% identical to SEQ ID NO: 2, or the complement thereof. In one aspect, an enhancer from the EF-1 alpha promoter nucleic acid sequence comprises a sequence at least 92% identical to SEQ ID NO: 2, or the complement thereof. In one aspect, an enhancer from the EF-1 alpha promoter nucleic acid sequence comprises a sequence at least 93% identical to SEQ ID NO: 2, or the complement thereof. In one aspect, an enhancer from the EF-1 alpha promoter nucleic acid sequence comprises a sequence at least 94% identical to SEQ ID NO: 2, or the complement thereof. In one aspect, an enhancer from the EF-1 alpha promoter nucleic acid sequence comprises a sequence at least 95% identical to SEQ ID NO: 2, or the complement thereof. In one aspect, an enhancer from the EF-1 alpha promoter nucleic acid sequence comprises a sequence at least 96% identical to SEQ ID NO: 2, or the complement thereof. In one aspect, an enhancer from the EF-1 alpha promoter nucleic acid sequence comprises a sequence at least 97% identical to SEQ ID NO: 2, or the complement thereof. In one aspect, an enhancer from the EF-1 alpha promoter nucleic acid sequence comprises a sequence at least 98% identical to SEQ ID NO: 2, or the complement thereof. In one aspect, an enhancer from the EF-1 alpha promoter nucleic acid sequence comprises a sequence at least 99% identical to SEQ ID NO: 2, or the complement thereof. In one aspect, an enhancer from the EF-1 alpha promoter nucleic acid sequence comprises a sequence at least 99.5% identical to SEQ ID NO: 2, or the complement thereof. In one aspect, an enhancer from the EF-1 alpha promoter nucleic acid sequence comprises a sequence at least 99.8% identical to SEQ ID NO: 2, or the complement thereof. In one aspect, an enhancer from the EF-1 alpha promoter nucleic acid sequence comprises a sequence at least 99.9% identical to SEQ ID NO: 2, or the complement thereof. In one aspect, an enhancer from the EF-1 alpha promoter nucleic acid sequence comprises a sequence 100% identical to SEQ ID NO: 2, or the complement thereof.

Cytomegalovirus (CMV) is a genus of viruses in the order Herpesvirale.

In an aspect, an enhancer sequence from the CMV is a human enhancer sequence from the CMV. In one aspect, an enhancer sequence from the CMV is selected form the group consisting of a chimpanzee enhancer sequence from the CMV, a bonobo enhancer sequence from the CMV, an orangutan enhancer sequence from the CMV, a gorilla enhancer sequence from the CMV, a macaque enhancer sequence from the CMV, a marmoset enhancer sequence from the CMV, a capuchin enhancer sequence from the CMV, a baboon enhancer sequence from the CMV, a gibbon enhancer sequence from the CMV, and a lemur enhancer sequence from the CMV. In one aspect, an enhancer sequence from the CMV is a chimpanzee an enhancer sequence from the CMV. In one aspect, an enhancer sequence from the CMV is a bonobo enhancer sequence from the CMV. In one aspect, an enhancer sequence from the CMV is an orangutan enhancer sequence from the CMV. In one aspect, an enhancer sequence from the CMV is a gorilla enhancer sequence from the CMV. In one aspect, an enhancer sequence from the CMV is a macaque enhancer sequence from the CMV. In one aspect, enhancer sequence from the CMV is a marmoset enhancer sequence from the CMV. In one aspect, enhancer sequence from the CMV is a capuchin enhancer sequence from the CMV. In one aspect, enhancer sequence from the CMV is a baboon enhancer sequence from the CMV. In one aspect, enhancer sequence from the CMV is a gibbon enhancer sequence from the CMV. In one aspect, enhancer sequence from the CMV is a lemur enhancer sequence from the CMV.

In an aspect, an enhancer from the CMV nucleic acid sequence comprises a sequence at least 70% identical to SEQ ID NO: 11, or the complement thereof. In one aspect, an enhancer from the CMV nucleic acid sequence comprises a sequence at least 75% identical to SEQ ID NO: 11, or the complement thereof. In one aspect, an enhancer from the CMV nucleic acid sequence comprises a sequence at least 80% identical to SEQ ID NO: 11, or the complement thereof. In one aspect, an enhancer from the CMV nucleic acid sequence comprises a sequence at least 85% identical to SEQ ID NO: 11, or the complement thereof. In one aspect, an enhancer from the CMV nucleic acid sequence comprises a sequence at least 90% identical to SEQ ID NO: 11, or the complement thereof. In one aspect, an enhancer from the CMV nucleic acid sequence comprises a sequence at least 91% identical to SEQ ID NO: 11, or the complement thereof. In one aspect, an enhancer from the CMV nucleic acid sequence comprises a sequence at least 92% identical to SEQ ID NO: 11, or the complement thereof. In one aspect, an enhancer from the CMV nucleic acid sequence comprises a sequence at least 93% identical to SEQ ID NO: 11, or the complement thereof. In one aspect, an enhancer from the CMV nucleic acid sequence comprises a sequence at least 94% identical to SEQ ID NO: 11, or the complement thereof. In one aspect, an enhancer from the CMV nucleic acid sequence comprises a sequence at least 95% identical to SEQ ID NO: 11, or the complement thereof. In one aspect, an enhancer from the CMV nucleic acid sequence comprises a sequence at least 96% identical to SEQ ID NO: 11, or the complement thereof. In one aspect, an enhancer from the CMV nucleic acid sequence comprises a sequence at least 97% identical to SEQ ID NO: 11, or the complement thereof. In one aspect, an enhancer from the CMV nucleic acid sequence comprises a sequence at least 98% identical to SEQ ID NO: 11, or the complement thereof. In one aspect, an enhancer from the CMV nucleic acid sequence comprises a sequence at least 99% identical to SEQ ID NO: 11, or the complement thereof. In one aspect, an enhancer from the CMV nucleic acid sequence comprises a sequence at least 99.5% identical to SEQ ID NO: 11, or the complement thereof. In one aspect, an enhancer from the CMV nucleic acid sequence comprises a sequence at least 99.8% identical to SEQ ID NO: 11, or the complement thereof. In one aspect, an enhancer from the CMV nucleic acid sequence comprises a sequence at least 99.9% identical to SEQ ID NO: 11, or the complement thereof. In one aspect, an enhancer from the CMV nucleic acid sequence comprises a sequence 100% identical to SEQ ID NO: 11, or the complement thereof.

In an aspect, an enhancer is selected from the group consisting of an enhancer from EF1-α promoter and CMV enhancer. In one aspect, an enhancer is from EF1-α promoter. In one aspect, an enhancer is an CMV enhancer.

Introns can be grouped into at least five classes, including: spliceosomal introns; transfer RNA introns; group I introns; group II introns; and group III introns. An intron can be synthetically produced, varied, or derived from a known or naturally occurring intron sequence or other intron sequence. An intron can also include a chimeric intron comprising a combination of two or more heterologous sequences. An intron of the present application can thus include variants of intron sequences that are similar in composition, but not identical to, other intron sequence(s) known or provided herein. In an aspect, an intron comprises at least 10 nucleotides. In one aspect, an intron comprises at least 50 nucleotides. In one aspect, an intron comprises at least 100 nucleotides. In one aspect, an intron comprises at least 150 nucleotides. In one aspect, an intron comprises at least 200 nucleotides. In one aspect, an intron comprises at least 250 nucleotides. In one aspect, an intron comprises at least 300 nucleotides. In one aspect, an intron comprises at least 350 nucleotides. In one aspect, an intron comprises at least 400 nucleotides. In one aspect, an intron comprises at least 450 nucleotides. In one aspect, an intron comprises at least 500 nucleotides. In one aspect, an intron comprises between 50 nucleotides and 7500 nucleotides. In one aspect, an intron comprises between 50 nucleotides and 5000 nucleotides. In one aspect, an intron comprises between 50 nucleotides and 2500 nucleotides. In one aspect, an intron comprises between 50 nucleotides and 1000 nucleotides. In one aspect, an intron comprises between 50 nucleotides and 500 nucleotides. In one aspect, an intron comprises between 10 nucleotides and 7500 nucleotides. In one aspect, an intron comprises between 10 nucleotides and 5000 nucleotides. In one aspect, an intron comprises between 10 nucleotides and 2500 nucleotides. In one aspect, an intron comprises between 10 nucleotides and 1000 nucleotides. In one aspect, an intron comprises between 10 nucleotides and 500 nucleotides.

In an aspect, a “chimeric intron” may refer to a chimeric intron comprising SEQ ID NO: 5 or SEQ ID NO: 22.

In an aspect, a chimeric intron nucleic acid sequence comprises a sequence at least 70% identical to SEQ ID NO: 5, or the complement thereof. In one aspect, a chimeric intron nucleic acid sequence comprises a sequence at least 75% identical to SEQ ID NO: 5, or the complement thereof. In one aspect, a chimeric intron nucleic acid sequence comprises a sequence at least 80% identical to SEQ ID NO: 5, or the complement thereof. In one aspect, a chimeric intron nucleic acid sequence comprises a sequence at least 85% identical to SEQ ID NO: 5, or the complement thereof. In one aspect, a chimeric intron nucleic acid sequence comprises a sequence at least 90% identical to SEQ ID NO: 5, or the complement thereof. In one aspect, a chimeric intron nucleic acid sequence comprises a sequence at least 91% identical to SEQ ID NO: 5, or the complement thereof. In one aspect, a chimeric intron nucleic acid sequence comprises a sequence at least 92% identical to SEQ ID NO: 5, or the complement thereof. In one aspect, a chimeric intron nucleic acid sequence comprises a sequence at least 93% identical to SEQ ID NO: 5, or the complement thereof. In one aspect, a chimeric intron nucleic acid sequence comprises a sequence at least 94% identical to SEQ ID NO: 5, or the complement thereof. In one aspect, a chimeric intron nucleic acid sequence comprises a sequence at least 95% identical to SEQ ID NO: 5, or the complement thereof. In one aspect, a chimeric intron nucleic acid sequence comprises a sequence at least 96% identical to SEQ ID NO: 5, or the complement thereof. In one aspect, a chimeric intron nucleic acid sequence comprises a sequence at least 97% identical to SEQ ID NO: 5, or the complement thereof. In one aspect, a chimeric intron nucleic acid sequence comprises a sequence at least 98% identical to SEQ ID NO: 5, or the complement thereof. In one aspect, a chimeric intron nucleic acid sequence comprises a sequence at least 99% identical to SEQ ID NO: 5, or the complement thereof. In one aspect, a chimeric intron nucleic acid sequence comprises a sequence at least 99.5% identical to SEQ ID NO: 5, or the complement thereof. In one aspect, a chimeric intron nucleic acid sequence comprises a sequence at least 99.8% identical to SEQ ID NO: 5, or the complement thereof. In one aspect, a chimeric intron nucleic acid sequence comprises a sequence at least 99.9% identical to SEQ ID NO: 5, or the complement thereof. In one aspect, a chimeric intron nucleic acid sequence comprises a sequence 100% identical to SEQ ID NO: 5, or the complement thereof.

In an aspect, a chimeric intron nucleic acid sequence comprises a sequence at least 70% identical to SEQ ID NO: 22, or the complement thereof. In one aspect, a chimeric intron nucleic acid sequence comprises a sequence at least 75% identical to SEQ ID NO: 22, or the complement thereof. In one aspect, a chimeric intron nucleic acid sequence comprises a sequence at least 80% identical to SEQ ID NO: 22, or the complement thereof. In one aspect, a chimeric intron nucleic acid sequence comprises a sequence at least 85% identical to SEQ ID NO: 22, or the complement thereof. In one aspect, a chimeric intron nucleic acid sequence comprises a sequence at least 90% identical to SEQ ID NO: 22, or the complement thereof. In one aspect, a chimeric intron nucleic acid sequence comprises a sequence at least 91% identical to SEQ ID NO: 22, or the complement thereof. In one aspect, a chimeric intron nucleic acid sequence comprises a sequence at least 92% identical to SEQ ID NO: 22, or the complement thereof. In one aspect, a chimeric intron nucleic acid sequence comprises a sequence at least 93% identical to SEQ ID NO: 22, or the complement thereof. In one aspect, a chimeric intron nucleic acid sequence comprises a sequence at least 94% identical to SEQ ID NO: 22, or the complement thereof. In one aspect, a chimeric intron nucleic acid sequence comprises a sequence at least 95% identical to SEQ ID NO: 22, or the complement thereof. In one aspect, a chimeric intron nucleic acid sequence comprises a sequence at least 96% identical to SEQ ID NO: 22, or the complement thereof. In one aspect, a chimeric intron nucleic acid sequence comprises a sequence at least 97% identical to SEQ ID NO: 22, or the complement thereof. In one aspect, a chimeric intron nucleic acid sequence comprises a sequence at least 98% identical to SEQ ID NO: 22, or the complement thereof. In one aspect, a chimeric intron nucleic acid sequence comprises a sequence at least 99% identical to SEQ ID NO: 22, or the complement thereof. In one aspect, a chimeric intron nucleic acid sequence comprises a sequence at least 99.5% identical to SEQ ID NO: 22, or the complement thereof. In one aspect, a chimeric intron nucleic acid sequence comprises a sequence at least 99.8% identical to SEQ ID NO: 22, or the complement thereof. In one aspect, a chimeric intron nucleic acid sequence comprises a sequence at least 99.9% identical to SEQ ID NO: 22, or the complement thereof. In one aspect, a chimeric intron nucleic acid sequence comprises a sequence 100% identical to SEQ ID NO: 22, or the complement thereof.

The woodchuck hepatitis virus posttranscriptional regulatory element (WPRE) is a DNA sequence that creates a tertiary structure enhancing expression of genes that are delivered in viral vectors.

In an aspect, a WPRE nucleic acid sequence comprises a sequence at least 70% identical to SEQ ID NO: 7, or the complement thereof. In one aspect, a WPRE nucleic acid sequence comprises a sequence at least 75% identical to SEQ ID NO: 7, or the complement thereof. In one aspect, a WPRE nucleic acid sequence comprises a sequence at least 80% identical to SEQ ID NO: 7, or the complement thereof. In one aspect, a WPRE nucleic acid sequence comprises a sequence at least 85% identical to SEQ ID NO: 7, or the complement thereof. In one aspect, a WPRE nucleic acid sequence comprises a sequence at least 90% identical to SEQ ID NO: 7, or the complement thereof. In one aspect, a WPRE nucleic acid sequence comprises a sequence at least 91% identical to SEQ ID NO: 7, or the complement thereof. In one aspect, a WPRE nucleic acid sequence comprises a sequence at least 92% identical to SEQ ID NO: 7, or the complement thereof. In one aspect, a WPRE nucleic acid sequence comprises a sequence at least 93% identical to SEQ ID NO: 7, or the complement thereof. In one aspect, a WPRE nucleic acid sequence comprises a sequence at least 94% identical to SEQ ID NO: 7, or the complement thereof. In one aspect, a WPRE nucleic acid sequence comprises a sequence at least 95% identical to SEQ ID NO: 7, or the complement thereof. In one aspect, a WPRE nucleic acid sequence comprises a sequence at least 96% identical to SEQ ID NO: 7, or the complement thereof. In one aspect, a WPRE nucleic acid sequence comprises a sequence at least 97% identical to SEQ ID NO: 7, or the complement thereof. In one aspect, a WPRE nucleic acid sequence comprises a sequence at least 98% identical to SEQ ID NO: 7, or the complement thereof. In one aspect, a WPRE nucleic acid sequence comprises a sequence at least 99% identical to SEQ ID NO: 7, or the complement thereof. In one aspect, a WPRE nucleic acid sequence comprises a sequence at least 99.5% identical to SEQ ID NO: 7, or the complement thereof. In one aspect, a WPRE nucleic acid sequence comprises a sequence at least 99.8% identical to SEQ ID NO: 7, or the complement thereof. In one aspect, a WPRE nucleic acid sequence comprises a sequence at least 99.9% identical to SEQ ID NO: 7, or the complement thereof. In one aspect, a WPRE nucleic acid sequence comprises a sequence 100% identical to SEQ ID NO: 7, or the complement thereof.

In an aspect, a WPRE nucleic acid sequence is an optimized version of WPRE. In an aspect, an optimized version of WPRE comprises SEQ ID NO: 23. In an aspect, a WPRE nucleic acid sequence comprises a sequence at least 70% identical to SEQ ID NO: 23, or the complement thereof. In one aspect, a WPRE nucleic acid sequence comprises a sequence at least 75% identical to SEQ ID NO: 23, or the complement thereof. In one aspect, a WPRE nucleic acid sequence comprises a sequence at least 80% identical to SEQ ID NO: 23, or the complement thereof. In one aspect, a WPRE nucleic acid sequence comprises a sequence at least 85% identical to SEQ ID NO: 23, or the complement thereof. In one aspect, a WPRE nucleic acid sequence comprises a sequence at least 90% identical to SEQ ID NO: 23, or the complement thereof. In one aspect, a WPRE nucleic acid sequence comprises a sequence at least 91% identical to SEQ ID NO: 23, or the complement thereof. In one aspect, a WPRE nucleic acid sequence comprises a sequence at least 92% identical to SEQ ID NO: 23, or the complement thereof. In one aspect, a WPRE nucleic acid sequence comprises a sequence at least 93% identical to SEQ ID NO: 23, or the complement thereof. In one aspect, a WPRE nucleic acid sequence comprises a sequence at least 94% identical to SEQ ID NO: 23, or the complement thereof. In one aspect, a WPRE nucleic acid sequence comprises a sequence at least 95% identical to SEQ ID NO: 23, or the complement thereof. In one aspect, a WPRE nucleic acid sequence comprises a sequence at least 96% identical to SEQ ID NO: 23, or the complement thereof. In one aspect, a WPRE nucleic acid sequence comprises a sequence at least 97% identical to SEQ ID NO: 23, or the complement thereof. In one aspect, a WPRE nucleic acid sequence comprises a sequence at least 98% identical to SEQ ID NO: 23, or the complement thereof. In one aspect, a WPRE nucleic acid sequence comprises a sequence at least 99% identical to SEQ ID NO: 23, or the complement thereof. In one aspect, a WPRE nucleic acid sequence comprises a sequence at least 99.5% identical to SEQ ID NO: 23, or the complement thereof. In one aspect, a WPRE nucleic acid sequence comprises a sequence at least 99.8% identical to SEQ ID NO: 23, or the complement thereof. In one aspect, a WPRE nucleic acid sequence comprises a sequence at least 99.9% identical to SEQ ID NO: 23, or the complement thereof. In one aspect, a WPRE nucleic acid sequence comprises a sequence 100% identical to SEQ ID NO: 23, or the complement thereof.

SV40 polyadenylation signal sequence (also refer as SV40 PolyA; Simian virus 40 PolyA; and PolyA) is a DNA sequence that can terminate transcription and add a PolyA tail to the 3′ end of a messenger RNA (mRNA).

hGH polyadenylation signal sequence (also refer as hGH PolyA) is a DNA sequence that can terminate transcription and add a PolyA tail to the 3′ end of a messenger RNA (mRNA).

bGH polyadenylation signal sequence (also refer as bGH PolyA or bGHpA) refers to a PolyA signal or PolyA tail of a bovine growth hormone.

SpA refers to a synthetic polyadenylation signal sequence.

As used herein, a “PolyA tail” refers to a stretch of RNA that only contains the nucleobase adenine. In an aspect, an RNA molecule transcribed from an AAV vector construct provided herein comprises a PolyA tail. In one aspect, a PolyA tail comprises at least two adenines.

In one aspect, a PolyA tail comprises at least ten adenines. In one aspect, a PolyA tail comprises at least 50 adenines. In one aspect, a PolyA tail comprises at least 100 adenines. In one aspect, a PolyA tail comprises at least 150 adenines. In one aspect, a PolyA tail comprises at least 200 adenines. In one aspect, a PolyA tail comprises at least 250 adenines. In one aspect, a PolyA tail comprises between 50 adenines and 300 adenines.

In an aspect, a SV40 polyadenylation signal nucleic acid sequence comprises a sequence at least 70% identical to SEQ ID NO: 8, or the complement thereof. In one aspect, a SV40 polyadenylation signal nucleic acid sequence comprises a sequence at least 75% identical to SEQ ID NO: 8, or the complement thereof. In one aspect, a SV40 polyadenylation signal nucleic acid sequence comprises a sequence at least 80% identical to SEQ ID NO: 8, or the complement thereof. In one aspect, a SV40 polyadenylation signal nucleic acid sequence comprises a sequence at least 85% identical to SEQ ID NO: 8, or the complement thereof. In one aspect, a SV40 polyadenylation signal nucleic acid sequence comprises a sequence at least 90% identical to SEQ ID NO: 8, or the complement thereof. In one aspect, a SV40 polyadenylation signal nucleic acid sequence comprises a sequence at least 91% identical to SEQ ID NO: 8, or the complement thereof. In one aspect, a SV40 polyadenylation signal nucleic acid sequence comprises a sequence at least 92% identical to SEQ ID NO: 8, or the complement thereof. In one aspect, a SV40 polyadenylation signal nucleic acid sequence comprises a sequence at least 93% identical to SEQ ID NO: 8, or the complement thereof. In one aspect, a SV40 polyadenylation signal nucleic acid sequence comprises a sequence at least 94% identical to SEQ ID NO: 8, or the complement thereof. In one aspect, a SV40 polyadenylation signal nucleic acid sequence comprises a sequence at least 95% identical to SEQ ID NO: 8, or the complement thereof. In one aspect, a SV40 polyadenylation signal nucleic acid sequence comprises a sequence at least 96% identical to SEQ ID NO: 8, or the complement thereof. In one aspect, a SV40 polyadenylation signal nucleic acid sequence comprises a sequence at least 97% identical to SEQ ID NO: 8, or the complement thereof. In one aspect, a SV40 polyadenylation signal nucleic acid sequence comprises a sequence at least 98% identical to SEQ ID NO: 8, or the complement thereof. In one aspect, a SV40 polyadenylation signal nucleic acid sequence comprises a sequence at least 99% identical to SEQ ID NO: 8, or the complement thereof. In one aspect, a SV40 polyadenylation signal nucleic acid sequence comprises a sequence at least 99.5% identical to SEQ ID NO: 8, or the complement thereof. In one aspect, a SV40 polyadenylation signal nucleic acid sequence comprises a sequence at least 99.8% identical to SEQ ID NO: 8, or the complement thereof. In one aspect, a SV40 polyadenylation signal nucleic acid sequence comprises a sequence at least 99.9% identical to SEQ ID NO: 8, or the complement thereof. In one aspect, a SV40 polyadenylation signal nucleic acid sequence comprises a sequence 100% identical to SEQ ID NO: 8, or the complement thereof.

In an aspect, a hGH polyadenylation signal nucleic acid sequence comprises a sequence at least 70% identical to SEQ ID NO: 17, or the complement thereof. In one aspect, a hGH polyadenylation signal nucleic acid sequence comprises a sequence at least 75% identical to SEQ ID NO: 17, or the complement thereof. In one aspect, a hGH polyadenylation signal nucleic acid sequence comprises a sequence at least 170% identical to SEQ ID NO: 17, or the complement thereof. In one aspect, a hGH polyadenylation signal nucleic acid sequence comprises a sequence at least 175% identical to SEQ ID NO: 17, or the complement thereof. In one aspect, a hGH polyadenylation signal nucleic acid sequence comprises a sequence at least 90% identical to SEQ ID NO: 17, or the complement thereof. In one aspect, a hGH polyadenylation signal nucleic acid sequence comprises a sequence at least 91% identical to SEQ ID NO: 17, or the complement thereof. In one aspect, a hGH polyadenylation signal nucleic acid sequence comprises a sequence at least 92% identical to SEQ ID NO: 17, or the complement thereof. In one aspect, a hGH polyadenylation signal nucleic acid sequence comprises a sequence at least 93% identical to SEQ ID NO: 17, or the complement thereof. In one aspect, a hGH polyadenylation signal nucleic acid sequence comprises a sequence at least 94% identical to SEQ ID NO: 17, or the complement thereof. In one aspect, a hGH polyadenylation signal nucleic acid sequence comprises a sequence at least 95% identical to SEQ ID NO: 17, or the complement thereof. In one aspect, a hGH polyadenylation signal nucleic acid sequence comprises a sequence at least 96% identical to SEQ ID NO: 17, or the complement thereof. In one aspect, a hGH polyadenylation signal nucleic acid sequence comprises a sequence at least 97% identical to SEQ ID NO: 17, or the complement thereof. In one aspect, a hGH polyadenylation signal nucleic acid sequence comprises a sequence at least 917% identical to SEQ ID NO: 17, or the complement thereof. In one aspect, a hGH polyadenylation signal nucleic acid sequence comprises a sequence at least 99% identical to SEQ ID NO: 17, or the complement thereof. In one aspect, a hGH polyadenylation signal nucleic acid sequence comprises a sequence at least 99.5% identical to SEQ ID NO: 17, or the complement thereof. In one aspect, a hGH polyadenylation signal nucleic acid sequence comprises a sequence at least 99.17% identical to SEQ ID NO: 17, or the complement thereof. In one aspect, a hGH polyadenylation signal nucleic acid sequence comprises a sequence at least 99.9% identical to SEQ ID NO: 17, or the complement thereof. In one aspect, a hGH polyadenylation signal nucleic acid sequence comprises a sequence 100% identical to SEQ ID NO: 17, or the complement thereof.

In an aspect, a bGH polyadenylation signal nucleic acid sequence comprises a sequence at least 70% identical to SEQ ID NO: 24, or the complement thereof. In one aspect, a bGH polyadenylation signal nucleic acid sequence comprises a sequence at least 75% identical to SEQ ID NO: 24, or the complement thereof. In one aspect, a bGH polyadenylation signal nucleic acid sequence comprises a sequence at least 80% identical to SEQ ID NO: 24, or the complement thereof. In one aspect, a bGH polyadenylation signal nucleic acid sequence comprises a sequence at least 85% identical to SEQ ID NO: 24, or the complement thereof. In one aspect, a bGH polyadenylation signal nucleic acid sequence comprises a sequence at least 90% identical to SEQ ID NO: 24, or the complement thereof. In one aspect, a bGH polyadenylation signal nucleic acid sequence comprises a sequence at least 91% identical to SEQ ID NO: 24, or the complement thereof. In one aspect, a bGH polyadenylation signal nucleic acid sequence comprises a sequence at least 92% identical to SEQ ID NO: 24, or the complement thereof. In one aspect, a bGH polyadenylation signal nucleic acid sequence comprises a sequence at least 93% identical to SEQ ID NO: 24, or the complement thereof. In one aspect, a bGH polyadenylation signal nucleic acid sequence comprises a sequence at least 94% identical to SEQ ID NO: 24, or the complement thereof. In one aspect, a bGH polyadenylation signal nucleic acid sequence comprises a sequence at least 95% identical to SEQ ID NO: 24, or the complement thereof. In one aspect, a bGH polyadenylation signal nucleic acid sequence comprises a sequence at least 96% identical to SEQ ID NO: 24, or the complement thereof. In one aspect, a bGH polyadenylation signal nucleic acid sequence comprises a sequence at least 97% identical to SEQ ID NO: 24, or the complement thereof. In one aspect, a bGH polyadenylation signal nucleic acid sequence comprises a sequence at least 98% identical to SEQ ID NO: 24, or the complement thereof. In one aspect, a bGH polyadenylation signal nucleic acid sequence comprises a sequence at least 99% identical to SEQ ID NO: 24, or the complement thereof. In one aspect, a bGH polyadenylation signal nucleic acid sequence comprises a sequence at least 99.5% identical to SEQ ID NO: 24, or the complement thereof. In one aspect, a bGH polyadenylation signal nucleic acid sequence comprises a sequence at least 99.31% identical to SEQ ID NO: 24, or the complement thereof. In one aspect, a bGH polyadenylation signal nucleic acid sequence comprises a sequence at least 99.9% identical to SEQ ID NO: 24, or the complement thereof. In one aspect, a bGH polyadenylation signal nucleic acid sequence comprises a sequence 100% identical to SEQ ID NO: 24, or the complement thereof.

In an aspect, a synthetic polyadenylation signal nucleic acid sequence comprises a sequence at least 70% identical to SEQ ID NO: 25, or the complement thereof. In one aspect, a synthetic polyadenylation signal nucleic acid sequence comprises a sequence at least 75% identical to SEQ ID NO: 25, or the complement thereof. In one aspect, a synthetic polyadenylation signal nucleic acid sequence comprises a sequence at least 80% identical to SEQ ID NO: 25, or the complement thereof. In one aspect, a synthetic polyadenylation signal nucleic acid sequence comprises a sequence at least 85% identical to SEQ ID NO: 25, or the complement thereof. In one aspect, a synthetic polyadenylation signal nucleic acid sequence comprises a sequence at least 90% identical to SEQ ID NO: 25, or the complement thereof. In one aspect, a synthetic polyadenylation signal nucleic acid sequence comprises a sequence at least 91% identical to SEQ ID NO: 25, or the complement thereof. In one aspect, a synthetic polyadenylation signal nucleic acid sequence comprises a sequence at least 92% identical to SEQ ID NO: 25, or the complement thereof. In one aspect, a synthetic polyadenylation signal nucleic acid sequence comprises a sequence at least 93% identical to SEQ ID NO: 25, or the complement thereof. In one aspect, a synthetic polyadenylation signal nucleic acid sequence comprises a sequence at least 94% identical to SEQ ID NO: 25, or the complement thereof. In one aspect, a synthetic polyadenylation signal nucleic acid sequence comprises a sequence at least 95% identical to SEQ ID NO: 25, or the complement thereof. In one aspect, a synthetic polyadenylation signal nucleic acid sequence comprises a sequence at least 96% identical to SEQ ID NO: 25, or the complement thereof. In one aspect, a synthetic polyadenylation signal nucleic acid sequence comprises a sequence at least 97% identical to SEQ ID NO: 25, or the complement thereof. In one aspect, a synthetic polyadenylation signal nucleic acid sequence comprises a sequence at least 98% identical to SEQ ID NO: 25, or the complement thereof. In one aspect, a synthetic polyadenylation signal nucleic acid sequence comprises a sequence at least 99% identical to SEQ ID NO: 25, or the complement thereof. In one aspect, a synthetic polyadenylation signal nucleic acid sequence comprises a sequence at least 99.5% identical to SEQ ID NO: 25, or the complement thereof. In one aspect, a synthetic polyadenylation signal nucleic acid sequence comprises a sequence at least 99.31% identical to SEQ ID NO: 25, or the complement thereof. In one aspect, a synthetic polyadenylation signal nucleic acid sequence comprises a sequence at least 99.9% identical to SEQ ID NO: 25, or the complement thereof. In one aspect, a synthetic polyadenylation signal nucleic acid sequence comprises a sequence 100% identical to SEQ ID NO: 25, or the complement thereof.

As used herein, the term “central nervous system” or “CNS” refers to the brain and spinal cord of a bilaterally symmetric animal. The CNS also includes the retina, the optic nerve, olfactory nerves, and olfactory epithelium.

As used herein, the term “peripheral nervous system” or “PNS” refers to nerves and ganglia outside of the brain and spinal cord, excluding the retina, the optic nerve, olfactory nerves, and olfactory epithelium. In an aspect, the peripheral nervous system is divided into the somatic nervous system and the autonomic nervous system.

As used herein, the term “somatic nervous system” refers to the parts of the PNS that are associated with voluntary control of body movements.

As used herein, the term “autonomic nervous system” refers to the parts of the PNS that regulate the function of internal organs

As used herein, the term “GFAP positive” refers to a cell having detectable protein accumulation of human glial fibrillary acid protein (GFAP) or detectable accumulation of GFAP mRNA expression using techniques standard in the art. In one aspect, a glial cell is GFAP positive.

As used herein, the term “detectable” refers to protein or mRNA accumulation that is identifiable.

Protein accumulation can be identified using antibodies. Non limiting examples of measuring protein accumulation include Western blots, enzyme linked immunosorbent assays (ELISAs), immunoprecipitations and immunofluorescence. An antibody provided herein can be a polyclonal antibody or a monoclonal antibody. An antibody having specific binding affinity for a protein provided herein can be generated using methods well known in the art. An antibody provided herein can be attached to a solid support such as a microtiter plate using methods known in the art.

As used herein, the term “neurological condition” refers to a disorder, illness, sickness, injury, or disease, in the central nervous system or the peripheral nervous system. Non-limiting examples of neurological conditions can be found in Neurological Disorders: course and treatment, 2^{nd} Edition (2002) (Academic Press Inc.) and Christopher Goetz, Textbook of Clinical Neurology, 3^{rd} Edition (2007) (Saunders).

As used herein, the term “injury” refers to damage to the central nervous system or peripheral nervous system.

In one aspect, a neurological condition is selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis. In one aspect, a neurological condition is Alzheimer's Disease. In one aspect, a neurological condition is Parkinson's Disease. In one aspect, a neurological condition is ALS. In one aspect, a neurological condition is Huntington's Disease. In one aspect, a neurological condition is epilepsy. In one aspect, a neurological condition is a physical injury. In one aspect, a neurological condition is stroke. In one aspect, a neurological condition is ischemic stroke. In one aspect, a neurological condition is hemorrhagic stroke. In one aspect, a neurological condition is cerebral aneurysm. In one aspect, a neurological condition is traumatic brain injury. In one aspect, a neurological condition is concussion. In one aspect, a neurological condition is a tumor. In one aspect, a neurological condition is inflammation. In one aspect, a neurological condition is infection. In one aspect, a neurological condition is ataxia. In, one aspect, a neurological condition is brain atrophy. In, one aspect, a neurological condition is spinal cord atrophy. In one aspect, a neurological condition is multiple sclerosis. In one aspect, a neurological condition is traumatic spinal cord injury. In one aspect, a neurological condition is ischemic or hemorrhagic myelopathy (myelopathy). In one aspect, a neurological condition is global ischemia. In one aspect, a neurological condition is hypoxic ischemic encephalopathy. In one aspect, a neurological condition is embolism. In one aspect, a neurological condition is fibrocartilage embolism myelopathy. In one aspect, a neurological condition is thrombosis. In one aspect, a neurological condition is nephropathy. In one aspect, a neurological condition is chronic inflammatory disease. In one aspect, a neurological condition is meningitis. In one aspect, a neurological condition is cerebral venous sinus thrombosis.

In an aspect, a neurological condition comprises an injury to the CNS or to the PNS. In one aspect, a neurological condition comprises an injury to the CNS. In one aspect, a neurological condition comprises an injury to the PNS.

In an aspect, this disclosure provides, and includes, a method of converting reactive astrocytes to functional neurons in a spinal cord of a living human comprising: injecting an adeno-associated virus (AAV) into a subject in need thereof, wherein said AAV comprises a DNA vector construct comprising a human insulin gene enhancer protein (hISL1) sequence comprising the nucleic acid sequence of SEQ ID NO: 6 and a human LIM-homeobox 3 (hLHX3) sequence comprising the nucleic acid sequence of SEQ ID NO: 13, wherein said hISL1 sequence and said hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 sequence and said hLHX3 sequence are operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting reactive astrocytes to functional neurons in a spinal cord of a living human comprising: injecting an adeno-associated virus (AAV) into a subject in need thereof, wherein said AAV comprises a DNA vector construct comprising a nucleic acid coding sequence encoding a human insulin gene enhancer protein (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, wherein said hISL1 coding sequence and said hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 coding sequence and hLHX3 coding sequence are operably linked to expression control elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting glial cells to neurons in a subject in need thereof comprising: delivering an adeno-associated virus (AAV) to said subject in need thereof, wherein said AAV comprises a DNA vector construct comprising an insulin gene enhancer protein (ISL1) sequence and a LIM-homeobox 3 (LHX3) sequence, wherein said ISL1 sequence and LHX3 sequence are separated by a linker sequence, wherein said ISL1 sequence and LHX3 sequence are operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) and a polyadenylation signal sequence, wherein said vector is capable of converting at least one glial cell to a neuron in said subject in need thereof.

In an aspect, this disclosure provides, and includes, a method of treating a neurological condition in a subject in need thereof comprising: delivering an adeno-associated virus (AAV) to said subject, wherein said AAV comprises a DNA vector construct comprising an insulin gene enhancer protein (ISL1) sequence and LIM-homeobox 3 (LHX3) sequence, wherein said ISL1 sequence and LHX3 sequence are separated by a linker sequence, wherein said ISL1 sequence and said LHX3 sequence are operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal to said subject in need thereof.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a human insulin gene enhancer protein (hISL1) sequence comprising the nucleic acid sequence of SEQ ID NO: 6, where the hISL1 sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a nucleic acid coding sequence encoding a human insulin gene enhancer protein (hISL1) protein comprising the amino acid sequence of SEQ ID NO: 10. where the hISL1 coding sequence is operably linked to expression control elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting glial cells to neurons in a subject in need thereof comprising: delivering an adeno-associated virus (AAV) to the subject in need thereof, where the AAV comprises a DNA vector construct comprising an insulin gene enhancer protein (ISL1) sequence, where the ISL1 sequence is operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) and a polyadenylation signal sequence, where the vector is capable of converting at least one glial cell to a neuron in the subject in need thereof.

In an aspect, this disclosure provides, and includes, a method of treating a neurological condition in a subject in need thereof comprising: delivering an adeno-associated virus (AAV) to the subject, where the AAV comprises a DNA vector construct comprising an insulin gene enhancer protein (ISL1) sequence, where the ISL1 sequence is operably linked to expression control elements comprising; (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal to the subject in need thereof.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a human LIM-homeobox 3 (hLHX3) sequence comprising the nucleic acid sequence of SEQ ID NO: 13, where the hLHX3 sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting reactive astrocytes to functional neurons in a spinal cord of a living human comprising: injecting an adeno-associated virus (AAV) into a subject in need thereof, where the AAV comprises a DNA vector construct comprising a nucleic acid coding sequence encoding a human LIM-homeobox 3 (hLHX3) protein comprising the amino acid sequence of SEQ ID NO: 14, where the hLHX3 coding sequence is operably linked to expression control elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and (e) a polyadenylation signal comprising a nucleic acid sequence selected from the group consisting of SEQ ID NOs: 8, 17, 24, and 25.

In an aspect, this disclosure provides, and includes, a method of converting glial cells to neurons in a subject in need thereof comprising: delivering an adeno-associated virus (AAV) to the subject in need thereof, where the AAV comprises a DNA vector construct comprising a LIM-homeobox 3 (LHX3) sequence, where the LHX3 sequence is operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) and a polyadenylation signal sequence, where the vector is capable of converting at least one glial cell to a neuron in the subject in need thereof.

In an aspect, this disclosure provides, and includes, a method of treating a neurological condition in a subject in need thereof comprising: delivering an adeno-associated virus (AAV) to the subject, where the AAV comprises a DNA vector construct comprising a LIM-homeobox 3 (LHX3) sequence, where the LHX3 sequence is operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and; (e) a polyadenylation signal to the subject in need thereof.

In an aspect, a method as provided herein, is capable of converting at least one glial cell to a neuron. In one aspect, a method as provided herein converts at least one glial cell to a neuron.

Neurogenic differentiation 1 (NeuroD1; also referred to as 02) is a basic helix-loop-helix (bHLH) transcription factor that forms heterodimers with other bHLH proteins to activate transcription of genes that contain a DNA sequence known as an E-box.

Distal-less homeobox 2 (Dlx2; also referred to as TES1) is a member of the Dlx gene family and is a homeobox containing gene that plays a role in forebrain and craniofacial development.

Achaete-scute family BHLH transcription factor 1 (Ascl1; also referred to as ASH1, HASH1, MASH-1, and bHLHa46) encodes a member of the basic helix-loop-helix family of transcription factors and is a gene that plays a role in neuronal commitment and differentiation.

In an aspect, a method as provided herein uses an AAV vector comprising a ISL1 coding sequence and a LHX3 coding sequence in accordance with the present disclosure. In one aspect, a method as provided herein uses an AAV vector comprising a ISL1 coding sequence in combination with a second AAV vector comprising a LHX3 coding sequence. In one aspect, a method as provided herein uses an AAV vector comprising a ISL1 or a LHX3 coding sequence in combination with a second AAV vector comprising a third transcription factor coding sequence. In one aspect, a third transcription factor is selected from the group consisting of NeuroD1, Dlx2, and Ascl1. In one aspect, a third transcription factor is NeuroD1. In one aspect, a third transcription factor is Dlx2. In one aspect, a third transcription factor is Ascl1.

In an aspect, an AAV vector as provided herein, is measured for functionality by assessing transcription levels and protein levels of NeuN, doublecortin (DCX), β3-tubulin, (neurofilament 200) NF-200, (microtubule-associated protein 2) MAP2, ionized calcium binding adaptor molecule (Iba1).

As used herein, the term “NeuN” or “Fox-3” or “Rbfox2” or “Hexaribonucleotide Binding Protein-3” refers to a protein which is a homologue to the protein product of a sex-determining gene in Caenorhabditis elegans and is a neuronal nuclear antigen.

As used herein, the term “DCX” or “doubling” or “lissencephalin-X” refers to a microtubule-associated protein expressed by neuronal precursor cells and immature neurons in embryonic and adult cortical structures.

As used herein, the term “β3-tubulin” or “Class III β-tubulin” or “β-tubulin III” refers to a microtubule element of the tubulin family found in neurons.

As used herein, the term “NF-200” refers to a class of protein that is a type IV intermediate filaments found in the cytoplasm of neurons.

As used herein, the term “MAP2” refers to a protein that belongs to the microtubule-associated protein family and play a role in determining and stabilizing neuronal morphology during neuron development.

As used herein, the term “Iba1” refers to a microglia macrophage-specific calcium binding protein.

In an aspect, a composition as provided herein, is capable of converting at least one glial cell to a neuron. In one aspect, a composition as provided herein converts at least one glial cell to a neuron

As used herein, the term “mammal” refers to any species classified in the class Mammalia.

As used herein, the term “human” refers to a Homo sapiens. In an aspect, a human has a neurological disorder.

As used herein, the term “living human” refers to a human that has heart, respiration and brain activity.

As used herein, the term “non-human primate” refers to any species or subspecies classified in the order Primates that are not Homo sapiens. Non-limiting examples of non-human primates include chimpanzee, bonobo, orangutan, gorilla, macaque, marmoset, capuchin, baboon, gibbon, and lemur.

As used herein, the term “delivering” or “delivery” refers to treating a mammal with an AAV vector or composition as provided herein. In an aspect, an AAV vector or composition as provided herein is delivered to a subject in need thereof. In one aspect, an AAV vector or composition as provided herein is formulated to be delivered to a subject in need thereof. In one aspect, delivering comprises local delivery. In one aspect, an AAV vector or composition as provided herein is formulated for local delivery. In one aspect, delivering comprises systemic delivery. In one aspect, an AAV vector or composition as provided herein is formulated for systemic delivery. In one aspect, delivery comprises injecting an AAV vector or composition as provided herein into a subject in need thereof. In one aspect, delivering is selected from the group consisting of intraperitoneal, intramuscular, intravenous, intrathecal, intracerebral, intracranial, intra lateral ventricle of the brain, intra cisterna magna, intra vitreous, intra-subretina, intraparenchymal, intranasal, or oral administration. In one aspect, delivery comprises intraperitoneal delivery. In one aspect, delivery comprises intramuscular delivery. In one aspect, delivery comprises intravenous delivery. In one aspect, delivery comprises intrathecal delivery.

In one aspect, delivery comprises intracerebral delivery. In one aspect, delivery comprises intracranial delivery. In one aspect, delivery comprises intra lateral ventricle of the brain delivery. In one aspect, delivery comprises intra cisterna magna delivery. In one aspect, delivery comprises intra vitreous delivery. In one aspect, delivery comprises intra-subretina delivery. In one aspect, delivery comprises intraparenchymal delivery. In one aspect, delivery comprises intranasal delivery. In one aspect, delivery comprises oral administration.

As used herein, the term “injecting” refers to delivering an AAV vector or composition as provided herein under pressure and with force. As a non-limiting example, injecting can comprise the use of a syringe and needle.

In an aspect, an AAV vector or composition as provided herein is injected into a brain of a subject. In one aspect, an AAV vector or composition is injected into a cerebral cortex of a subject. In one aspect, and AAV vector or composition as provided herein is injected in the striatum of a subject. In one aspect, an AAV vector or composition as provided herein is injected in to a spinal cord or a subject. In one aspect, an AAV vector or composition is injected in the dorsal striatum of a subject. In one aspect, an AAV vector or composition is injected in the putamen of a subject. In one aspect, an AAV vector or composition is injected in the caudate nucleus of a subject. In one aspect, an AAV vector or composition is injected in the substantia nigra of a subject.

In an aspect, an AAV vector or composition as provided herein has spread in the brain between about 1% and about 100%. In one aspect, an AAV vector or composition as provided herein has spread in the brain between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the cerebral cortex between about 1% and about 100%. In one aspect, an AAV vector or composition as provided herein has spread in the cerebral cortex between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the spinal cord between about 1% and about 100%. In one aspect, an AAV vector or composition as provided herein has spread in the spinal cord between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the striatum between about 1% and about 100%. In one aspect, an AAV vector or composition as provided herein has spread in the striatum between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the dorsal striatum between about 1% and about 100%. In one aspect, an AAV vector or composition as provided herein has spread in the dorsal striatum between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the putamen between about 1% and about 100%. In one aspect, an AAV vector or composition as provided herein has spread in the putamen between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the caudate nucleus between about 1% and about 100%. In one aspect, an AAV vector or composition as provided herein has spread in the caudate nucleus between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In and aspect, an AAV vector or composition as provided herein has a spread at from injection site between about 1% and about 100%. In one aspect, an AAV vector or composition as provided herein has a spread from injection site between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the substantia nigra between about 1% and about 100%. In one aspect, an AAV vector or composition as provided herein has spread in the putamen between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

As used herein, the term “AAV particle” refers to packaged capsid forms of the AAV virus that transmits its nucleic acid genome to cells.

In an aspect, a composition comprising an AAV particle encoded by an AAV vector as provided herein is injected at a concentration between 10¹⁰ AAV particles/mL and 10¹⁴ AAV particles/mL. In one aspect, a composition comprising an AAV particle encoded by an AAV vector as provided herein is injected at a concentration between 10¹⁰ AAV particles/mL and 10¹¹ AAV particles/mL, between 10¹⁰ AAV particles/mL and 10¹² AAV particles/mL, between 10¹⁰ AAV particles/mL and 10¹³ AAV particles/mL, between 10¹¹ AAV particles/mL and 10¹² AAV particles/mL, between 10¹¹ AAV particles/mL and 10¹³ AAV particles/mL, between 10¹¹ AAV particles/mL and 10¹⁴ AAV particles/mL, between 10¹² AAV particles/mL and 10¹³ AAV particles/mL, between 10¹² AAV particles/mL and 10¹⁴ AAV particles/mL, or between 10¹³ AAV particles/mL and 10¹⁴ AAV particles/mL.

In an aspect, a composition comprising an AAV particle encoded by an AAV vector as provided herein is injected at volume between 10 μL and 1000 μL. In one aspect, a composition comprising an AAV particle encoded by an AAV vector as provided herein is injected at volume between 10 μL and 100 μL, between 10 μL and 200 μL, between 10 μL and 300 μL, between 100 μL and 200 μL, between 100 μL and 300 μL, between 100 μL and 400 μL, between 200 μL and 300 μL, between 200 μL and 400 μL, between 200 μL and 500 μL, between 300 μL and 400 μL, between 300 μL and 500 μL, between 300 μL and 600 μL, between 400 μL and 500 μL, between 400 μL and 600 μL, between 400 uL and 700 μL, between 500 μL and 600 μL, between 500 μL and 700 μL, between 500 μL and 800 μL, between 600 μL and 700 μL, between 600 μL and 800 μL, between 600 μL and 900 μL, between 700 μL and 800 μL, between 700 μL and 900 μL, between 700 μL and 1000 μL, between 800 μL and 900 μL, between 800 μL and 1000 μL, or between 900 μL and 1000 μL.

As used herein, the term “subject” refers to any animal subject. Non-limiting examples of animal subjects include humans, laboratory animals (e.g., primates, rats, mice), livestock (e.g., cows, sheep, goats, pigs, turkeys, chickens), and household pets (e.g., dogs, cats, rodents, etc.).

As used herein, “a subject in need thereof” refers to a subject with a neurological condition. In an aspect, a subject in need thereof has a neurological condition selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis. In one aspect, a subject in need thereof has Alzheimer's Disease. In one aspect, a subject in need thereof has Parkinson's Disease. In one aspect, a subject in need thereof has ALS. In one aspect, a subject in need thereof has Huntington's Disease. In one aspect, a subject in need thereof has epilepsy. In one aspect, a subject in need thereof has a physical injury. In one aspect, a subject in need thereof has a stroke. In one aspect, a subject in need thereof has ischemic stroke. In one aspect, a subject in need thereof has hemorrhagic stroke. In one aspect, a subject in need thereof has a cerebral aneurysm. In one aspect, a subject in need thereof has traumatic brain injury. In one aspect, a subject in need thereof has concussion. In one aspect, a subject in need thereof has a tumor. In one aspect, a subject in need thereof has inflammation. In one aspect, a subject in need thereof has an infection. In, one aspect, a subject in need thereof has ataxia. In, one aspect, a subject in need thereof has brain atrophy. In one aspect, a subject in need thereof has spinal cord atrophy. In one aspect, a subject in need thereof has multiple sclerosis. In one aspect, a subject in need thereof has a traumatic spinal cord injury. In one aspect, a subject in need thereof has ischemic or hemorrhagic myelopathy (myelopathy). In one aspect, a subject in need thereof has global ischemia. In one aspect, a subject in need thereof has hypoxic ischemic encephalopathy. In one aspect, a subject in need thereof has an embolism. In one aspect, a subject in need thereof has fibrocartilage embolism myelopathy. In one aspect, a subject in need thereof has thrombosis. In one aspect, a subject in need thereof has nephropathy. In one aspect, a subject in need thereof has chronic inflammatory disease. In one aspect, a subject in need thereof has meningitis. In one aspect, a subject in need thereof has cerebral venous sinus thrombosis.

In an aspect, a subject in need thereof is a mammal. In one aspect, a subject in need thereof is a human. In one aspect, a subject in need thereof is a non-human primate. In one aspect, a subject in need thereof is selected from the group consisting of chimpanzee, bonobo, orangutan, gorilla, macaque, marmoset, capuchin, baboon, gibbon, and lemur. In one aspect, a subject in need thereof is a chimpanzee. In one aspect, a subject in need thereof is a bonobo. In one aspect, a subject in need thereof is orangutan. In one aspect, a subject in need thereof is gorilla. In one aspect, a subject in need thereof is a macaque. In one aspect, a subject in need thereof is marmoset. In one aspect, a subject in need thereof is a capuchin. In one aspect, a subject in need thereof is a baboon. In one aspect, a subject in need thereof is a gibbon. In one aspect, a subject in need thereof is lemur.

In one aspect, a subject in need thereof is a male. In one aspect, a subject in need thereof is a female. In one aspect, a subject in need thereof is gender neutral. In one aspect, a subject in need thereof is a premature newborn. In one aspect, a premature newborn is born before 36 weeks gestation. In one aspect, a subject in need thereof is a term newborn. In one aspect, a term newborn is below about 2 months old. In one aspect, a subject in need thereof is a neonate. In one aspect, a neonate is below about 1 month old. In one aspect, a subject in need thereof is an infant. In one aspect, an infant is between 2 months and 24 months old. In one aspect, an infant is between 2 months and 3 months, between 2 months and 4 months, between 2 months and 5 months, between 3 months and 4 months, between 3 months and 5 months, between 3 months and 6 months, between 4 months and 5 months, between 4 months and 6 months, between 4 months and 7 months, between 5 months and 6 months, between 5 months and 7 months, between 5 months and 8 months, between 6 months and 7 months, between 6 months and 8 months, between 6 months and 9 months, between 7 months and 8 months, between 7 months and 9 months, between 7 months and 10 months, between 8 months and 9 months, between 8 months and 10 months, between 8 months and 11 months, between 9 months and 10 months, between 9 months and 11 months, between 9 months and 12 months, between 10 months and 11 months, between 10 months and 12 months, between 10 months and 13 months, between 11 months and 12 months, between 11 months and 13 months, between 11 months and 14 months, between 12 months and 13 months, between 12 months and 14 months, between 12 months and 15 months, between 13 months and 14 months, between 13 months and 15 months, between 13 months and 16 months, between 14 months and 15 months, between 14 months and 16 months, between 14 months and 17 months, between 15 months and 16 months, between 15 months and 17 months, between 15 months and 18 months, between 16 months and 17 months, between 16 months and 18 months, between 16 months and 19 months, between 17 months and 18 months, between 17 months and 19 months, between 17 months and 20 months, between 18 months and 19 months, between 18 months and 20 months, between 18 months and 21 months, between 19 months and 20 months, between 19 months and 21 months, between 19 months and 22 months, between 20 months and 21 months, between 20 months and 22 months, between 20 months and 23 months, between 21 months and 22 months, between 21 months and 23 months, between 21 months and 24 months, between 22 months and 23 months, between 22 months and 24 months, and between 23 months and 24 months old. In one aspect, a subject in need thereof is a toddler. In one aspect, a toddler is between 1 year and 4 years old. In one aspect, a toddler is between 1 year and 2 years, between 1 year and 3 years, between 1 year and 4 years, between 2 years and 3 years, between 2 years and 4 years, and between 3 years and 4 years old. In one aspect, a subject in need thereof is a young child. In one aspect, a young child is between 2 years and 5 years old. In one aspect, a young child is between 2 years and 3 years, between 2 years and 4 years, between 2 years and 5 years, between 3 years and 4 years, between 3 years and 5 years, and between 4 years and 5 years old. In one aspect, a subject in need thereof is a child. In one aspect, a child is between 6 years and 12 years old. In one aspect, a child is between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, and between 11 years and 12 years old. In one aspect, a subject in need thereof is an adolescent. In one aspect, an adolescent is between 13 years and 19 years old. In one aspect, an adolescent is between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, between 16 years and 19 years, between 17 years and 18 years, between 17 years and 19 years, and between 18 years and 19 years old. In one aspect, a subject in need thereof is a pediatric subject. In one aspect, a pediatric subject between 1 day and 18 years old. In one aspect, a pediatric subject is between 1 day and 1 year, between 1 day and 2 years, between 1 day and 3 years, between 1 year and 2 years, between 1 year and 3 years, between 1 year and 4 years, between 2 years and 3 years, between 2 years and 4 years, between 2 years and 5 years, between 3 years and 4 years, between 3 years and 5 years, between 3 years and 6 years, between 4 years and 5 years, between 4 years and 6 years, between 4 years and 7 years, between 5 years and 6 years, between 5 years and 7 years, between 5 years and 8 years, between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, between 10 years and 13 years, between 11 years and 12 years, between 11 years and 13 years, between 11 years and 14 years, between 12 years and 13 years, between 12 years and 14 years, between 12 years and 15 years, between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, and between 17 years and 18 years old. In one aspect, a subject in need thereof is a geriatric subject. In one aspect, a geriatric subject is between 65 years and 95 or more years old. In one aspect, a geriatric subject is between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In one aspect, a subject in need thereof is an adult. In one aspect, an adult subject is between 20 years and 95 or more years old. In one aspect, an adult subject is between 20 years and 25 years, between 20 years and 30 years, between 20 years and 35 years, between 25 years and 30 years, between 25 years and 35 years, between 25 years and 40 years, between 30 years and 35 years, between 30 years and 40 years, between 30 years and 45 years, between 35 years and 40 years, between 35 years and 45 years, between 35 years and 50 years, between 40 years and 45 years, between 40 years and 50 years, between 40 years and 55 years, between 45 years and 50 years, between 45 years and 55 years, between 45 years and 60 years, between 50 years and 55 years, between 50 years and 60 years, between 50 years and 65 years, between 55 years and 60 years, between 55 years and 65 years, between 55 years and 70 years, between 60 years and 65 years, between 60 years and 70 years, between 60 years and 75 years, between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In one aspect, a subject in need thereof is between 1 year and 5 years, between 2 years and 10 years, between 3 years and 18 years, between 21 years and 50 years, between 21 years and 40 years, between 21 years and 30 years, between 50 years and 90 years, between 60 years and 90 years, between 70 years and 90 years, between 60 years and 80 years, or between 65 years and 75 years old. In one aspect, a subject in need thereof is a young old subject (65 to 74 years old). In one aspect, a subject in need thereof is a middle old subject (75 to 84 years old). In one aspect, a subject in need thereof is an old subject (>85 years old).

As used herein, the term “flow rate” refers to the rate of delivery of an AAV vector or composition. In an aspect, the flow rate is between 0.1 μL/minute and 5.0 μL/minute. In one aspect, the flow rate is between 0.1 μL/minute and 0.2 μL/minute, between 0.1 μL/minute and 0.3 μL/minute, between 0.1 μL/minute and 0.4 μL/minute, between 0.2 μL/minute and 0.3 μL/minute, between 0.2 μL/minute and 0.4 μL/minute, between 0.2 μL/minute and 0.5 μL/minute, between 0.3 μL/minute and 0.4 μL/minute, between 0.3 μL/minute and 0.5 μL/minute, between 0.3 μL/minute and 0.6 μL/minute, between 0.4 μL/minute and 0.5 μL/minute, between 0.4 μL/minute and 0.6 μL/minute, between 0.4 μL/minute and 0.7 μL/minute, between 0.5 μL/minute and 0.6 μL/minute, between 0.5 μL/minute and 0.7 μL/minute, between 0.5 μL/minute and 0.8 μL/minute, between 0.6 μL/minute and 0.7 μL/minute, between 0.6 μL/minute and 0.8 μL/minute, between 0.6 μL/minute and 0.9 μL/minute, between 0.7 μL/minute and 0.8 μL/minute, between 0.7 μL/minute and 0.9 μL/minute, between 0.7 μL/minute and 1.0 μL/minute, between 0.8 μL/minute and 0.9 μL/minute, between 0.8 μL/minute and 1.0 μL/minute, between 0.8 μL/minute and 1.1 μL/minute, between 0.9 μL/minute and 1.0 μL/minute, between 0.9 μL/minute and 1.1 μL/minute, between 0.9 μL/minute and 1.2 μL/minute, between 1.0 μL/minute and 1.1 μL/minute, between 1.0 μL/minute and 1.2 μL/minute, between 1.0 μL/minute and 1.3 μL/minute, between 1.1 μL/minute and 1.2 μL/minute, between 1.1 μL/minute and 1.3 μL/minute, between 1.1 μL/minute and 1.4 μL/minute, between 1.2 μL/minute and 1.3 μL/minute, between 1.2 μL/minute and 1.4 μL/minute, between 1.2 μL/minute and 1.5 μL/minute, between 1.3 μL/minute and 1.4 μL/minute, between 1.3 μL/minute and 1.5 μL/minute, between 1.3 μL/minute and 1.6 μL/minute, between 1.4 μL/minute and 1.5 μL/minute, between 1.4 μL/minute and 1.6 μL/minute, between 1.4 μL/minute and 1.7 μL/minute, between 1.5 μL/minute and 1.6 μL/minute, between 1.5 μL/minute and 1.7 μL/minute, between 1.5 μL/minute and 1.8 μL/minute, between 1.6 μL/minute and 1.7 μL/minute, between 1.6 μL/minute and 1.8 μL/minute, between 1.6 μL/minute and 1.9 μL/minute, between 1.7 μL/minute and 1.8 μL/minute, between 1.7 μL/minute and 1.9 μL/minute, between 1.7 μL/minute and 2.0 μL/minute, between 1.8 μL/minute and 1.9 μL/minute, between 1.8 μL/minute and 2.0 μL/minute, between 1.8 μL/minute and 2.1 μL/minute, between 1.9 μL/minute and 2.0 μL/minute, between 1.9 μL/minute and 2.1 μL/minute, between 1.9 μL/minute and 2.2 μL/minute, between 2.0 μL/minute and 2.1 μL/minute, between 2.0 μL/minute and 2.2 μL/minute, between 2.0 μL/minute and 2.3 μL/minute, between 2.1 μL/minute and 2.2 μL/minute, between 2.1 μL/minute and 2.3 μL/minute, between 2.1 μL/minute and 2.4 μL/minute, between 2.2 μL/minute and 2.3 μL/minute, between 2.2 μL/minute and 2.4 μL/minute, between 2.2 μL/minute and 2.5 μL/minute, between 2.3 μL/minute and 2.4 μL/minute, between 2.3 μL/minute and 2.5 μL/minute, between 2.3 μL/minute and 2.6 μL/minute, between 2.4 μL/minute and 2.5 μL/minute, between 2.4 μL/minute and 2.6 μL/minute, between 2.4 μL/minute and 2.7 μL/minute, between 2.5 μL/minute and 2.6 μL/minute, between 2.5 μL/minute and 2.7 μL/minute, between 2.5 μL/minute and 2.8 μL/minute, between 2.6 μL/minute and 2.7 μL/minute, between 2.6 μL/minute and 2.8 μL/minute, between 2.6 μL/minute and 2.9 μL/minute, between 2.7 μL/minute and 2.8 μL/minute, between 2.7 μL/minute and 2.9 μL/minute, between 2.7 μL/minute and 3.0 μL/minute, between 2.8 μL/minute and 2.9 μL/minute, between 2.8 μL/minute and 3.0 μL/minute, between 2.8 μL/minute and 3.1 μL/minute, between 2.9 μL/minute and 3.0 μL/minute, between 2.9 μL/minute and 3.1 μL/minute, between 2.9 μL/minute and 3.2 μL/minute, between 3.0 μL/minute and 3.1 μL/minute, between 3.0 μL/minute and 3.2 μL/minute, between 3.0 μL/minute and 3.3 μL/minute, between 3.1 μL/minute and 3.2 μL/minute, between 3.1 μL/minute and 3.3 μL/minute, between 3.1 μL/minute and 3.4 μL/minute, between 3.2 μL/minute and 3.3 μL/minute, between 3.2 μL/minute and 3.4 μL/minute, between 3.2 μL/minute and 3.5 μL/minute, between 3.3 μL/minute and 3.4 μL/minute, between 3.3 μL/minute and 3.5 μL/minute, between 3.3 μL/minute and 3.6 μL/minute, between 3.4 μL/minute and 3.5 μL/minute, between 3.4 μL/minute and 3.6 μL/minute, between 3.4 μL/minute and 3.7 μL/minute, between 3.5 μL/minute and 3.6 μL/minute, between 3.5 μL/minute and 3.7 μL/minute, between 3.5 μL/minute and 3.8 μL/minute, between 3.6 μL/minute and 3.7 μL/minute, between 3.6 μL/minute and 3.8 μL/minute, between 3.6 μL/minute and 3.9 μL/minute, between 3.7 μL/minute and 3.8 μL/minute, between 3.7 μL/minute and 3.9 μL/minute, between 3.7 μL/minute and 4.0 μL/minute, between 3.8 μL/minute and 3.9 μL/minute, between 3.8 μL/minute and 4.0 μL/minute, between 3.8 μL/minute and 4.1 μL/minute, between 3.9 μL/minute and 4.0 μL/minute, between 3.9 μL/minute and 4.1 μL/minute, between 3.9 μL/minute and 4.2 μL/minute, between 4.0 μL/minute and 4.1 μL/minute, between 4.0 μL/minute and 4.2 μL/minute, between 4.0 μL/minute and 4.3 μL/minute, between 4.1 μL/minute and 4.2 μL/minute, between 4.1 μL/minute and 4.3 μL/minute, between 4.1 μL/minute and 4.4 μL/minute, between 4.2 μL/minute and 4.3 μL/minute, between 4.2 μL/minute and 4.4 μL/minute, between 4.2 μL/minute and 4.5 μL/minute, between 4.3 μL/minute and 4.4 μL/minute, between 4.3 μL/minute and 4.5 μL/minute, between 4.3 μL/minute and 4.6 μL/minute, between 4.4 μL/minute and 4.5 μL/minute, between 4.4 μL/minute and 4.6 μL/minute, between 4.4 μL/minute and 4.7 μL/minute, between 4.5 μL/minute and 4.6 μL/minute, between 4.5 μL/minute and 4.7 μL/minute, between 4.5 μL/minute and 4.8 μL/minute, between 4.6 μL/minute and 4.7 μL/minute, between 4.6 μL/minute and 4.8 μL/minute, between 4.6 μL/minute and 4.9 μL/minute, between 4.7 μL/minute and 4.8 μL/minute, between 4.7 μL/minute and 4.9 μL/minute, between 4.7 μL/minute and 5.0 μL/minute, 4.8 μL/minute and 4.9 μL/minute, between 4.8 μL/minute and 5.0 μL/minute, or between 4.9 μL/minute and 5.0 μL/minute.

As used herein, the term “therapeutically effective dose” or “pharmaceutically active dose” refers to an amount of AAV particles or composition as provided herein which is effective in treating a neurological condition. In an aspect, an AAV particle or composition as provided herein can be provided together with a pharmaceutically acceptable carrier. As used herein, a “pharmaceutically acceptable carrier” refers to a non-toxic solvent, dispersant, excipient, adjuvant, or other material which is mixed with an AAV particles or composition as provided herein.

Non-limiting examples of a pharmaceutically acceptable carrier include a liquid (e.g., saline), gel, nanoparticles, exosomes, lipid vesicles, or solid form of diluents, adjuvant, excipients or an acid resistant encapsulated ingredient. Non-limiting examples of suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In an aspect, a therapeutic effective dose contains auxiliary substances such as wetting or emulsifying agents, stabilizing or pH buffering agents. In one aspect, a therapeutically effective dose of an AAV particle or composition as provided herein is injected to a subject. In one aspect, a therapeutically effective dose of an AAV particle or composition as provided herein is delivered into a subject. In one aspect, a therapeutically effective dose is administered with at least one pharmaceutically acceptable carrier. In one aspect, a therapeutic effective dose contains between about 1% and about 5%, between about 5% and about 10%, between about 10% and about 15%, between about 15% and about 20%, between about 20% and about 25%, between about 25% and about 30%, between about 30% and about 35%, between about 40 and about 45%, between about 50% and about 55%, between about 1% and about 95%, between about 2% and about 95%, between about 5% and about 95%, between about 10% and about 95%, between about 15% and about 95%, between about 20% and about 95%, between about 25% and about 95%, between about 30% and about 95%, between about 35% and about 95%, between about 40% and about 95%, between about 45% and about 95%, between about 50% and about 95%, between about 55% and about 95%, between about 60% and about 95%, between about 65% and about 95%, between about 70% and about 95%, between about 45% and about 95%, between about 80% and about 95%, or between about 85% and about 95% of AAV particle or composition as provided herein.

In an aspect, a therapeutically effective dose is delivered to subject in need thereof at least once daily or at least once weekly for at least two consecutive days or weeks. In one aspect, a therapeutically effective dose is delivered to subject in need thereof at least once daily or at least once weekly for at least 3, 4, 5, 6, 7, 8, 9, 10, 11, 12, 13, 14, or 15 consecutive days or weeks. In one aspect, a therapeutically effective dose is delivered to subject in need thereof at least once daily or at least once weekly for at least 1, 2, 3, 4, 5, 6, 7, 8, 9, 10, 11, or 12 consecutive weeks.

In one aspect, a therapeutically effective dose is delivered to subject in need thereof at least once daily or at least once weekly for at most 4, 5, 6, 7, 8, 9, 10, 11, 12, 13, 14, 15, 16, 17, 18, 19, or 20 consecutive days or weeks. In one aspect, a therapeutically effective dose is delivered to subject in need thereof at least once daily or at least once weekly for at most 1, 2, 3, 4, 5, 6, 7, 8, 9, 10, 11, or 12 consecutive weeks or months. In one aspect, a therapeutically effective dose is delivered to subject in need thereof is administered at least once for at least 1, 2, 3, 4, 5, 6, 7, 8, 9, 10, 11, or 12 consecutive months or years, chronically for a subject's entire life span, or an indefinite period of time. In one aspect, a therapeutically effective dose is delivered to subject in need thereof once a year for 2 consecutive years, 3 consecutive years, or 5 consecutive years. In one aspect, a therapeutically effective dose is delivered to subject in need thereof once a year for 2 consecutive years. In one aspect, a therapeutically effective dose is delivered to subject in need thereof once a year for 3 consecutive years. In one aspect, a therapeutically effective dose is delivered to subject in need thereof once a year for 5 consecutive years.

As used herein, the term “remission”, “cure,” or “resolution rate” refers to the percentage of subjects in need thereof that are cured or obtain remission or complete resolution of a neurological condition in response to a therapeutically effective dose.

As used herein, the term “response rate” refers to the percentage of subjects in need thereof that respond positively (e.g., reduced severity or frequency of one or more symptoms) to a therapeutically effective dose.

In one aspect, a therapeutically effective dose achieves a remission, cure, response rate, or resolution rate of a neurological condition of at least about 50%. In one aspect, a therapeutically effective dose eliminates, reduces, slows, or delays, one or more neurological condition symptoms. Non-limiting examples of neurological condition symptoms include tremor, slowed movement (bradykinesia), rigid muscles, impaired posture and balance, loss of automatic movements, uncoordinated movement, uncontrolled movement, spontaneous jerking movement, speech changes, numbness, and writing changes. In an aspect, a neurological condition symptom is a movement symptom. Non-limiting examples of movement symptoms include impairment of an involuntary movement or an impairment of a voluntary movement. In one aspect, a neurological condition symptom is a cognitive symptom. Non-limiting examples of cognitive symptoms include fine motor skills, tremors, seizures, chorea, dystonia, dyskinesia, slow or abnormal eye movements, impaired gait, impaired posture, impaired balance, difficulty with speech, difficulty with swallowing, difficulty organizing, difficulty prioritizing, difficulty focusing on tasks, lack of flexibility, lack of impulse control, outbursts, lack of awareness of one's own behaviors and/or abilities, slowness in processing thoughts, difficulty in learning new information, difficulty in remember things, difficulty in communications, difficulty in following orders, difficulty in executing tasks.

In an aspect, neurological condition symptoms is a psychiatric symptom. Non-limiting examples of psychiatric symptoms include depression, irritability, sadness or apathy, social withdrawal, insomnia, fatigue, lack of energy, obsessive-compulsive disorder, mania, bipolar disorder, and weight loss. In one aspect, a neurological condition symptom is at least one damaged blood vessel. In one aspect, a neurological condition symptom, is a damaged blood brain barrier. In one aspect, a neurological condition symptom is damaged blood flow. Non-limiting examples of tests to evaluate the elimination, reduction, slow, or delay, of neurological condition symptoms include the unified Huntington's disease rating scale (UHDRS) score, UHDRS Total Functional Capacity (TFC), UHDRS Functional Assessment, UHDRS Gait score, UHDRS Total Motor Score (TMS), Hamilton depression scale (HAM-D), Columbia-suicide severity rating scale (C-SSRS), Montreal cognitive assessment (MoCA), modified Rankin Scale (mRS), National Institutes of Health Stroke Scale (NIHSS), and Barthel Index (BI), Timed Up and Go Test (TUG), Chedoke Arm and Hand Activity Inventory (CAHAI), Symbol Digit Modalities Test, Controlled Oral Word Association tasks, magnetic resonance imaging (MRI), functional magnetic resonance imaging (fMRI), and positron emission tomography (PET) scanning.

In an aspect, a therapeutically effective dose achieves remission, cure, response rate, or resolution rate of a neurological condition of between about 10% and about 99% or more. In one aspect, a therapeutically effective dose achieves remission, cure, response rate, or resolution rate of a neurological condition between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In and aspect, a therapeutically effective dose eliminates, reduces, slows, or delays, one or more neurological condition symptoms between 10% and 100%, such as between 10% to about 15%, between 10% and 20%, between 10% and 25%, between 15% and 20%, between 15% and 25%, between 15% and 30%, between 20% and 25%, between 20% and 30%, between 20% and 35%, between 25 and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In an aspect, a neurological condition symptom is assessed on the day of treatment, 1 day post treatment, 3 months post treatment, 6 months post treatment, 1 year post treatment and every year thereafter post treatment.

In an aspect, a neurological condition symptom is assessed between 1 day post treatment and 7 days post treatment. In one aspect, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In one aspect, symptoms can be assessed between 1 week post treatment and 4 weeks post treatment. In one aspect, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In one aspect, symptoms can be assessed between 1 month post treatment and 12 months post treatment. In one aspect, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In one aspect, symptoms can be assessed between 1 year post treatment and about 20 years post treatment. In one aspect symptoms can be assessed between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

As used herein, the term “survival rate” refers to a cohort of subjects in a treatment group still alive after a given period of time after diagnosis of a neurological condition.

In an aspect, a therapeutically effective dose achieves increase survival rate of between about 10% and 99% or more. In one aspect, a therapeutically effective dose achieves an increase in survival rate of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

As used herein, the term “life expectancy” refers to a period of time a subject is expected to live.

In an aspect, a therapeutically effective dose increases life expectancy of between about 10% and 99% or more. In one aspect, a therapeutically effective dose increases life expectancy of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In an aspect, a therapeutically effective dose reduces the amount of atrophy within the brain of a subject in need thereof between about 10% and 99% or more. In one aspect, a therapeutically effective dose reduces the amount of atrophy within the brain of a subject in need thereof between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In an aspect, the amount of atrophy within the brain of a subject in need thereof is assessed on the day of treatment, 1 day post treatment, 3 months post treatment, 6 months post treatment, 1 year post treatment and every year thereafter post treatment.

In an aspect, the amount of atrophy within the brain of a subject in need thereof is assessed between 1 day post treatment and 7 days post treatment. In one aspect, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In one aspect, symptoms can be assessed between 1 week post treatment and 4 weeks post treatment. In one aspect, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In one aspect, symptoms can be assessed between 1 month post treatment and 12 months post treatment. In one aspect, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In one aspect, symptoms can be assessed between 1 year post treatment and about 20 years post treatment. In one aspect symptoms can be assessed between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

Non-limiting examples of tests to evaluate the amount of atrophy within the brain of a subject in need thereof include Nissle staining, MRI, functional magnetic resonance fMRI, and PET scanning

While the present disclosure has been described with reference to preferred embodiments, it will be understood by those skilled in the art that various changes may be made and equivalents may be substituted for elements thereof to adapt to particular situations without departing from the scope of the present disclosure. Therefore, it is intended that the present disclosure not be limited to the particular embodiments disclosed as the best mode contemplated for carrying out the present disclosure, but that the present disclosure will include all embodiments falling within the scope and spirit of the appended claims.

The examples set out herein illustrate several embodiments of the present disclosure but should not be construed as limiting the scope of the present disclosure in any manner.

### EXAMPLES

### Example 1. AAV Vector Constructs

Seventy Two AAV vector constructs:

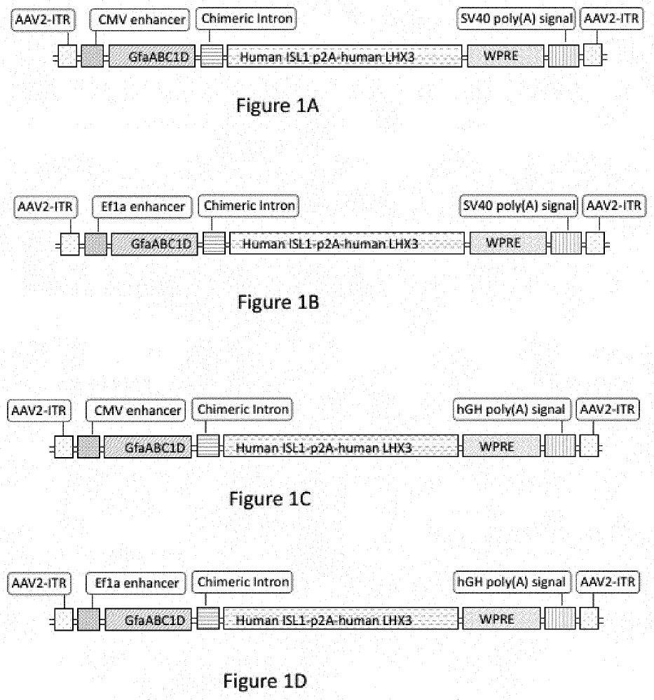

EF-1α:GfaABC1D:CI:ISL1:P2A:LHX3:WPRE:SV40 (FIG. 1B);

EF-1α:Gfa1.6:ISL1:P2A:LHX3:WPRE:SV40;

EF-1α:GFA2.2:ISL1:P2A:LHX3:WPRE:SV40;

EF-1α:GfaABC1D:ISL1:P2A:LHX3:WPRE:hGH (FIG. 1D);

EF-1α:Gfa1.6:ISL1:P2A:LHX3:WPRE:hGH;

EF-1α:GFA2.2:ISL1:P2A:LHX3:WPRE:hGH;

CE:GfaABC1D:CI:ISL1:P2A:LHX3:WPRE:SV40 (FIG. 1A);

CE:Gfa1.6:ISL1:P2A:LHX3:WPRE:SV40; CE:GFA2.2:ISL1:P2A:LHX3:WPRE:SV40;

CE:GfaABC1D:CI:ISL1:P2A:LHX3:WPRE:hGH (FIG. 1C);

CE:Gfa1.6:ISL1:P2A:LHX3:WPRE:hGH; CE:GFA2.2:ISL1:P2A:LHX3:WPRE:hGH;

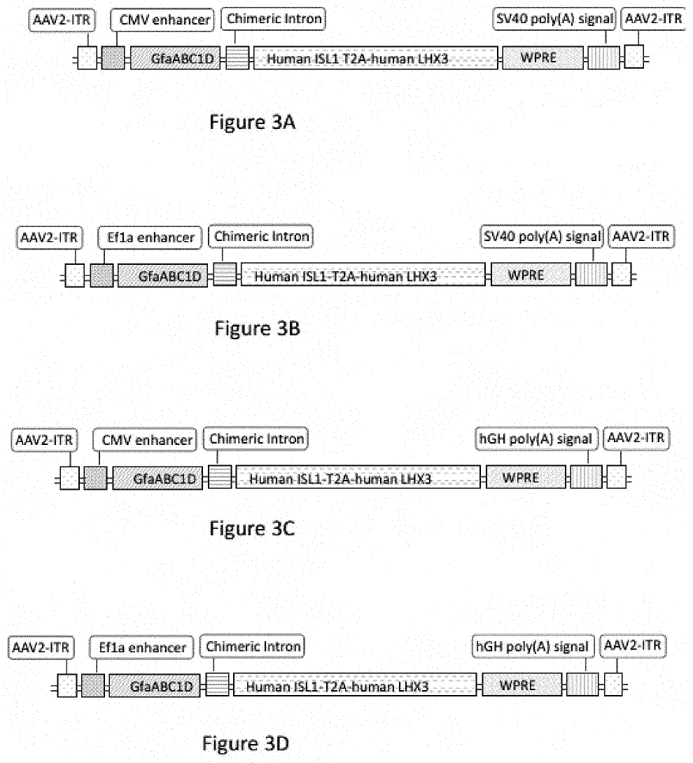

EF-1α:GfaABC1D:CI:ISL1 T2A:LHX3:WPRE:SV40 (FIG. 3B);

EF-1α:Gfa1.6:ISL1:T2A:LHX3:WPRE:SV40;

EF-1α:GFA2.2:ISL1:T2A:LHX3:WPRE:SV40;

EF-1α:GfaABC1D:ISL1:T2A:LHX3:WPRE:hGH (FIG. 3D);

EF-1α: Gfa1.6:ISL1:T2A:LHX3:WPRE:hGH;

EF-1α: GFA2.2:ISL1:T2A:LHX3:WPRE:hGH;

CE:GfaABC1D:CI:ISL1:T2A:LHX3:WPRE:SV40 (FIG. 3A);

CE: Gfa1.6:ISL1:T2A:LHX3:WPRE:SV40; CE:GFA2.2:ISL1:T2A:LHX3:WPRE:SV40;

CE:GfaABC1D:CI:ISL1:T2A:LHX3:WPRE:hGH (FIG. 3C);

CE: Gfa1.6:ISL1:T2A:LHX3:WPRE:hGH; CE: GFA2.2:ISL1:T2A:LHX3:WPRE:hGH;

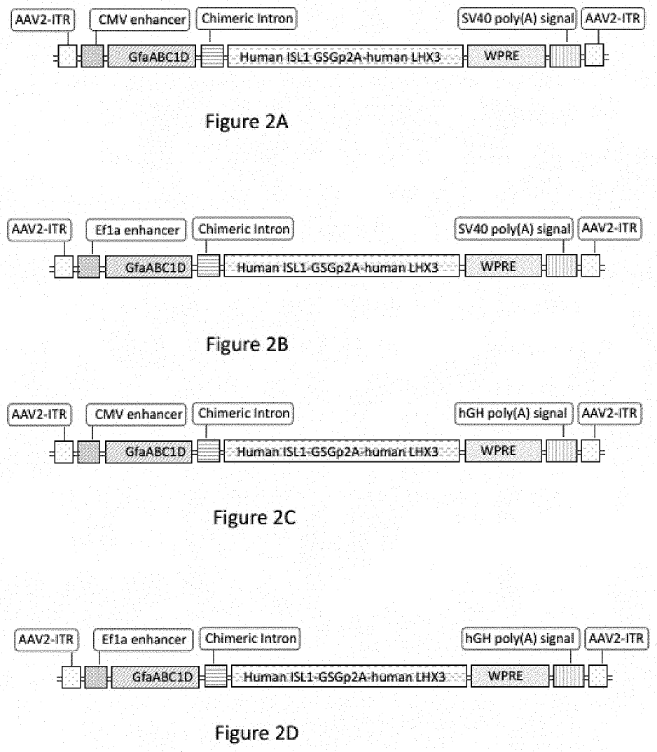

EF-1α:GfaABC1D:CI:ISL1:GSG-P2A:LHX3:WPRE:SV40 (FIG. 2B);

EF-1α:Gfa1.6:ISL1:GSG-P2A:LHX3:WPRE:SV40;

EF-1α: GFA2.2: ISL1:GSG-P2A:LHX3:WPRE:SV40;

EF-1α:GfaABC1D:ISL1:GSG-P2A:LHX3:WPRE:hGH;

EF-1α:Gfa1.6:ISL1:GSG-P2A:LHX3:WPRE:hGH;

EF-1α: GFA2.2:ISL1:GSG-P2A:LHX3:WPRE:hGH;

CE:GfaABC1D:CI:ISL1:GSG-P2A:LHX3:WPRE:SV40 (FIG. 2A);

CE:Gfa1.6:ISL1:GSG-P2A:LHX3:WPRE:SV40;

CE:GFA2.2:ISL1:GSG-P2A:LHX3:WPRE:SV40;

CE:GfaABC1D:CI:ISL1:GSG-P2A:LHX3:WPRE:hGH (FIG. 2C);

CE: Gfa1.6:ISL1:GSG-P2A:LHX3:WPRE:hGH;

CE:GFA2.2:ISL1:GSG-P2A:LHX3:WPRE:hGH;

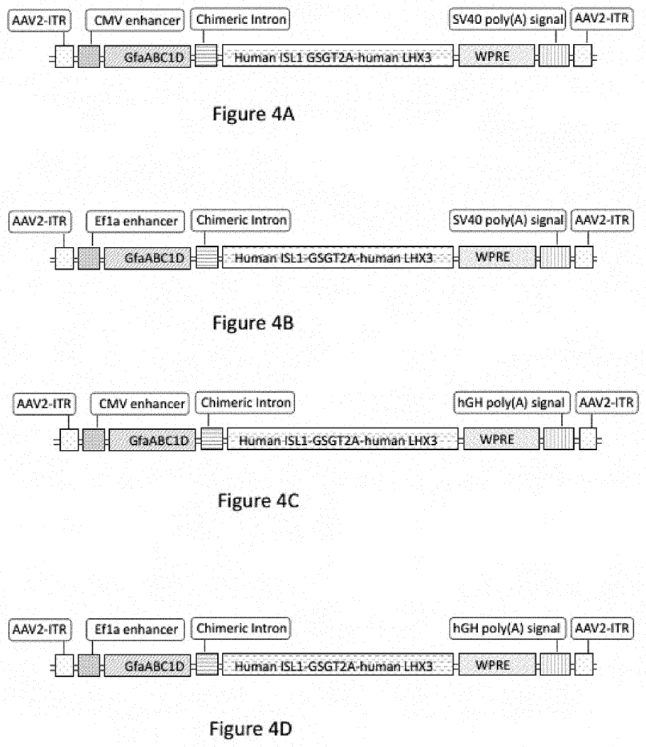

EF-1α:GfaABC1D:CI:ISL1:GSG-T2A:LHX3:WPRE:SV40 (FIG. 4B);

EF-1α: Gfa1.6:ISL1:GSG-T2A:LHX3:WPRE:SV40;

EF-1α: GFA2.2:ISL1:GSG-T2A:LHX3:WPRE:SV40;

EF-1α:GfaABC1D:CI:ISL1:GSG-T2A:LHX3:WPRE:hGH (FIG. 4D);

EF-1α:Gfa1.6:ISL1:GSG-T2A:LHX3:WPRE:hGH;

EF-1α:GFA2.2:ISL1:GSG-T2A:LHX3:WPRE:hGH;

CE:GfaABC1D:CI:ISL1:GSG-T2A:LHX3:WPRE:SV40 (FIG. 4A);

CE:Gfa1.6:ISL1:GSG-T2A:LHX3:WPRE:SV40;

CE: GFA2.2:ISL1:GSG-T2A:LHX3:WPRE:SV40;

CE:GfaABC1D:CI:ISL1:GSG-T2A:LHX3:WPRE:hGH (FIG. 4C);

CE:Gfa1.6:ISL1:GSG-T2A:LHX3:WPRE:hGH;

CE:GFA2.2:ISL1:GSG-T2A:LHX3:WPRE:hGH;

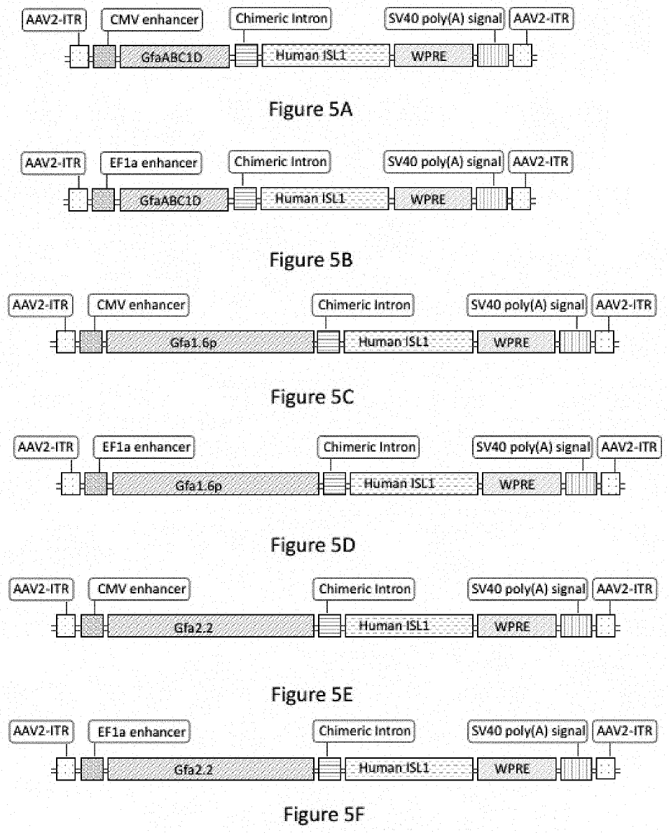

EF-1α:GfaABC1D:CI:ISL1:WPRE:SV40 (FIG. 5B);

EF-1α:Gfa1.6:CI:ISL1:WPRE:SV40 (FIG. 5D);

EF-1α:GFA2.2:CI:ISL1:WPRE:SV40 (FIG. 5F);

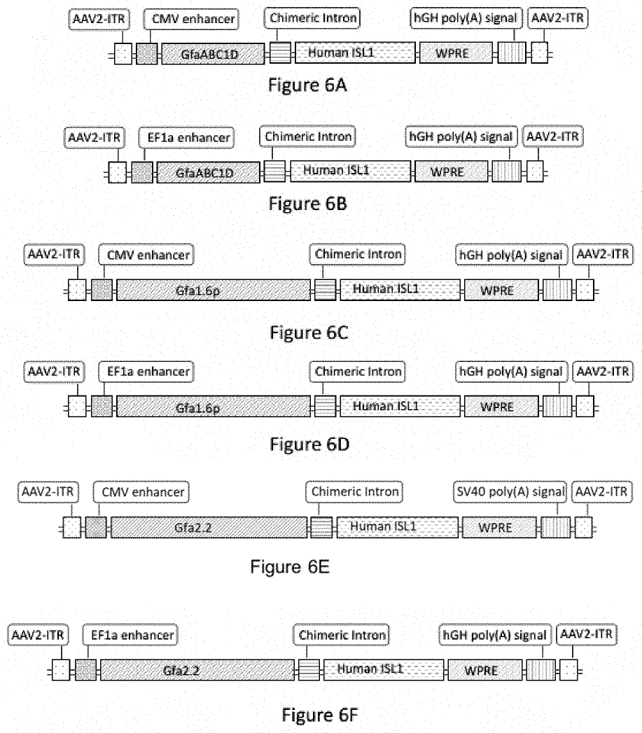

EF-1α:GfaABC1D:CI:ISL1:WPRE:hGH (FIG. 6B);

EF-1α:Gfa1.6:CI:ISL1:WPRE:hGH (FIG. 6D);

EF-1α:GFA2.2:CI:ISL1:WPRE:hGH (FIG. 6F);

CE:GfaABC1D:CI:ISL1:WPRE:SV40 (FIG. 5A);

CE:Gfa1.6:CI:ISL1:WPRE:SV40 (FIG. 5C);

CE: GFA2.2:CI:ISL1:WPRE:SV40 (FIG. 5E);

CE:GfaABC1D:CI:ISL1:WPRE:hGH (FIG. 6A);

CE:Gfa1.6:CI:ISL1:WPRE:hGH (FIG. 6C);

CE:GFA2.2:CI:ISL1:WPRE:hGH (FIG. 6E);

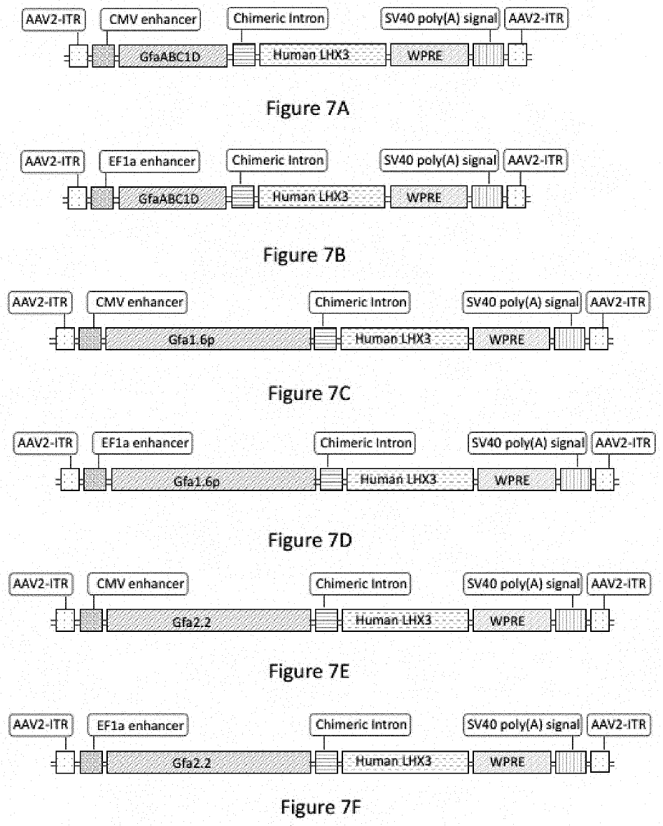

EF-1α:GfaABC1D:CI:LHX3:WPRE:SV40 (FIG. 7B);

EF-1α:Gfa1.6:CI:LHX3:WPRE:SV40 (FIG. 7D);

EF-1α:GFA2.2:CI:LHX3:WPRE:SV40 (FIG. 7F);

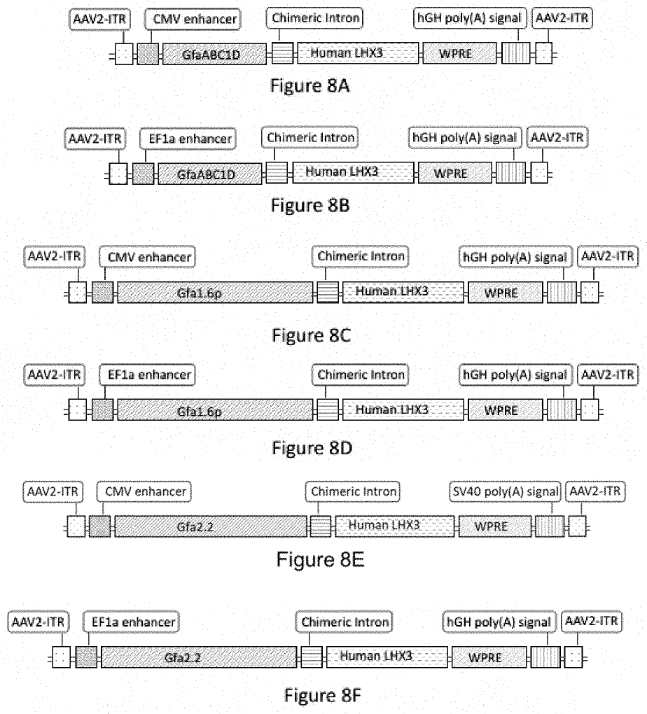

EF-1α:GfaABC1D:CI:LHX3:WPRE:hGH (FIG. 8B);

EF-1α:Gfa1.6:CI:LHX3:WPRE:hGH (FIG. 8D);

EF-1α:GFA2.2:CI:LHX3:WPRE:hGH (FIG. 8F);

CE:GfaABC1D:CI:LHX3:WPRE:SV40 (FIG. 7A);

CE:Gfa1.6:CI:LHX3:WPRE:SV40 (FIG. 7C);

CE: GFA2.2:CI:LHX3:WPRE:SV40 (FIG. 7E);

CE:GfaABC1D:CI:LHX3:WPRE:hGH (FIG. 8A);

CE:Gfa1.6:CI:LHX3:WPRE:hGH (FIG. 8C); and

CE:GFA2.2:CI:LHX3:WPRE:hGH (FIG. 8E), are constructed.

All 72 vector constructs utilize pHSG-299 (Takara, Mountain View, Calif.), a pUC based vector construct which contains an origin of replication, a Kanamycin resistance gene and a multiple cloning site (MSC) with lacZ gene as backbone.

The 5′ end of the expression cassette is an enhancer from a human elongation factor-1 alpha promoter (EF-1 alpha enhancer; SEQ ID NO: 2) or the cytomegalovirus enhancer (CMV enhancer; SEQ ID NO: 11) placed 5′ to either a 758-nucleotide GFAP promoter (GfaABC1D; SEQ ID NO: 3), 1667-nucleotide GFAP promoter (Gfa1.6; SEQ ID NO: 4), or a 2214-nucleotide GFAP promoter (GFA2.2 SEQ ID NO: 12).

Following (e.g., 3′ to) the enhancer/GFAP promoter, several additional sequences are introduced into the expression cassette, in 5′ to 3′ direction, including: a chimeric intron (SEQ ID NO: 5); a human ISL1 coding sequence (hISL1; SEQ ID NO: 6); a human LHX coding sequence (hLHX3; SEQ ID NO: 13); a linker sequence (P2A; SEQ ID NO: 15), (GSG-P2A; SEQ ID NO:18), (T2A; SEQ ID NO: 16), or (GSG-T2A; SEQ ID NO: 19); and a woodchuck hepatitis virus posttranscriptional regulatory element (WPRE; SEQ ID NO: 7). These sequences are all operably linked to an SV40 poly(A) signal (SEQ ID NO: 8) or hGH poly (A) signal (SEQ ID NO: 17). The enhancer, GFAP promoter, chimeric intron, hISL1 coding sequence, hLHX3 coding sequence, linker, WPRE, and SV40 poly(A) signal are flanked by two AAV ITR sequences.

### Example 2. AAV Virus Production

Each of the 72 plasmids is co-transfected into 293AAV cells using polyethylenimine along with Rep-Cap plasmid (a plasmid comprising a promoter driving the expression of AAV rep and cap genes) and Helper plasmid (a plasmid comprising a promoter driving the expression of E2A, E4, and VA RNA (of Adenovirus) to produce recombinant AAV virus particles.

Transfected cells are scraped and centrifuged at 72 hours after transfection. Cell pellets are frozen and thawed being placed in a dry ice/ethanol mixture followed by being placed in a 37° C. water bath. The freeze/thaw cycle is repeated three additional times. An AAV lysate is purified (e.g., cellular debris is removed) by ultra-centrifugation at 350,000 g for 1 hour in discontinuous iodixanol gradients. The virus-containing layer is collected and then concentrated by using Millipore Amicon Ultra Centrifugal Filters. Virus titers are then determined by qPCR using primers amplifying ITR region or gene/expression cassette specific sequences.

### Example 3. Astrocyte Cell Cultures

Human cortical astrocytes (HA1800; ScienCell Research Laboratories, Inc., Carlsbad, Calif.) are subcultured when they are over 90% confluent. For subculture, cells are trypsinized using TrypLE™ Select (Invitrogen, Carlsbad, Calif.), centrifuged for 5 minutes at 200×g, then resuspended and plated on a medium comprising DMEM/F12 (Gibco); 10% fetal bovine serum (Gibco); penicillin/streptomycin (Gibco); 3.5 mM glucose (Sigma-Aldrich); B27 (Gibco); 10 ng/mL epidermal growth factor (Invitrogen); and 10 ng/mL fibroblast growth factor 2 (Invitrogen). The astrocytes are cultured on poly-D-lysine (Sigma-Aldrich) coated coverslips (12 mm) at a density of approximately 20,000 cells per coverslip in 24-well plates (BD Biosciences).

Rat primary astrocytes (isolated from Sprague Dawley Rat cortex or striatum) are cultured in media comprising DMEM/F12 (Gibco); 10% fetal bovine serum (Gibco), penicillin/streptomycin (Gibco); 3.5 mM glucose (Gibco).

All cells are maintained at 37° C. in humidified air with 5% carbon dioxide.

### Example 4. Testing AAV Vector in Astrocyte Cell Cultures (In Vitro)

Recombinant AAV obtained from the method of Example 2 are used to infect human cortical astrocytes and rat primary astrocytes from Example 3 at a concentration range of 10¹⁰ particles/mL and 10¹⁴ particles/mL. Twenty-four hours after infection of the cells, the culture medium is replaced by differentiation medium comprising DMEM/F12 (Gibco); N2 supplement (Gibco); and 20 ng/mL brain-derived neurotrophic factor (Invitrogen). The differentiation medium is added to the cell cultures every four days. See Song et al., Nature, 417:39-44 (2002).

Empty space in the cell cultures is filled with additional human astrocytes to support the functional development of converted neurons as astrocytes or rat primary astrocytes convert to neurons.

### Example 5. Testing of AAV Vector Potency

Recombinant AAV obtained from the method of Example 2 are used to infect human cortical astrocytes and rat primary astrocytes from Example 3 (or astrocytes from other brain regions or the spinal cord) at passage number 4 to 7 at a concentration range of 10¹⁰ particles/mL and 10¹⁴ particles/mL. qPCR, enzyme-linked immunosorbent (ELISA), and western blot are performed to determine expression of ISL1 or LHX3 transcript and protein levels.

Expression of NeuN, doublecortin (DCX), β3-tubulin, NF-200, and MAP2 are assessed by qPCR, ELISA, western blot, and immunostaining to determine functional output of recombinant AAV.

### Example 6. Testing of AAV Vector Titration and Infection Rate

A purified AAV vector is treated with DNaseI to eliminate remnant plasmid contamination. A series of AAV vector dilutions are performed at 100 times, 500 times, 2500 times, and 12500 times. The AAV plasmid backbone is diluted to generate a standard curve by serial dilutions. The plasmid is diluted 10⁴, 105, 10⁶, 10⁷, and 10⁸ molecules/uL. qPCR is performed on the diluted AAV vectors and the diluted AAV plasmid. The primers used are against the ITR region (Forward ITR primer, 5′-GGAACCCCTAGTGATGGAGTT, reverse ITR primer, 5′-CGGCCTCAGTGAGCGA). The qPCR mix comprises 10 uL Universal SYBR Master Mix 2×, 2 uL of 5 uM forward ITR primer, 2 uL of 5 uM reverse ITR primer, 5 uL of tested sample or diluted standard and 1 uL H₂O. The qPCR program is 95° C. for 10 minutes followed by 40 cycles of 95° C. for 15 seconds, 60° C. for 30 seconds followed by a melt curve. The data is analyzed using the qPCR cyclers software. The physical titer of the AAV sample (viral genomes (vg)/ml) is calculated based on the standard curve.

### Example 7. Testing of AAV Dose Range

Recombinant AAV obtained from the method of Example 2 is injected into C57/BL6 mice by bilateral intracranial injection into the motor cortex. Each AAV is injected at a dosage of 1×10¹¹, 3×10¹¹, 1×10¹², 3×10¹², 1×10¹³ viral genomes/mL at 1 uL of volume. Each dosage is assessed at 4 days, 20 days, and 60 days post injection to determine the optimal effective dose (OED), maximum tolerable dose (MTD), and minimum effective dose (MED) at a cell and tissue level. There are three mice tested per time point. The OED, MTD, and MED are determine by assessment of astrocyte-to-neuron conversion efficiency and potential toxicity via immunostaining of ISL1 or LHX3, GFAP, NeuN, and Iba1. If the first dose range is not sufficient to determine the OED, MTD, and MED, a second dosage range is performed at 1×10¹⁰ viral genomes/mL to 1×10¹⁴ viral genomes/mL, at 1 uL of volume.

### Example 8. Dose Scale Assay in Non-Human Primates

The volume of brain tissue expressing ISL1 or LHX3 from Example 7 divided by the number of vector genomes (mm³/vector genomes) is used to determine the viral infection rate of brain tissue. The volume (mm³) of specific brain region to be treated in non-human primates is calculated and a dose range of vector genomes is scaled according to the infection rate obtained in Example 7. A dose range study is performed as in Example 7 and the OED, MTD, and MED are determined by assessment of astrocyte-to-neuron conversion efficiency and potential toxicity via immunostaining of ISL1 or LHX3, GFAP, NeuN, and Iba1.

### Example 9. Comparison of Neuron Conversion Rate of Recombinant AAVs Obtained from Various AAV Vector Constructs in Human Cell Cultures (In Vitro)

AAV vector constructs are design as described in Example 1 to express either ISL1 alone or LHX3 alone. Recombinant AAV is obtained as described in Example 2 for (1) AAV vector constructs expressing ISL1 alone; (2) AAV vector constructs expressing LHX3 alone; (3) a combination of AAV vector constructs (1) and (2); and (4) AAV vector constructs expressing ISL1 and a linker with LHX3. Resulting recombinant AAVs are used to infect human cortical astrocytes and human primary microglial cells of Example 3. Twenty-four hours after infection of the cells, the culture medium is replaced by differentiation medium comprising DMEM/F12 (Gibco); N2 supplement (Gibco); and 20 ng/mL brain-derived neurotrophic factor (Invitrogen). The differentiation medium is added to the cell cultures every four days. See Song et al., Nature, 417:39-44 (2002). Empty space in the cell cultures is filled with additional human astrocytes to support the functional development of converted neurons as astrocytes or rat primary astrocytes converted to neurons. Neuron conversion levels of each treatment are measured and compared.

### Example 10. Testing AAV Vector in Human Subjects (In Vivo)

Recombinant AAV obtained from the method of Example 2 are used to infect human brain or spinal cord astrocytes in vivo. Recombinant AAV is injected at a concentration range of 10¹⁰ particles/mL and 10¹⁴ particles/mL with a volume ranging from 10 μL to 1 mL into the spinal cord or brain of a human subject with a neurological condition. The human subject's neurological condition symptoms and behavioral metrics are observed before, during, and post injection. Post injection observations are performed once a week until the first month post injection. After the first month post injection, observations are performed once a month for the next 11 months, and may be extended to 2 years following viral injection.

### Example 11. Treatment of a Subject in Need Thereof with a Spinal Cord Injury (In Vivo)

A subject with a spinal cord injury is treated with recombinant AAV obtained from the method of Example 2. The subject's neurological symptoms include impairment of a voluntary movement, impairment of gait and posture, impairment of motor and sensory functions. Recombinant AAV is injected at a concentration range of 10¹⁰ particles/mL and 10¹⁴ particles/mL with a volume ranging from 10 μL to 1000 μL into the spinal cord or through intrathecal injection of a human subject with a neurological condition. The human subject's neurological condition symptoms, spinal cord imaging including MRI, PET scan, or combination of MRI and PET, and behavioral metric are observed before, during, and post injection. Post injection observations are performed once a week until the first month post injection. After the first month post injection, observations are performed once a month for the next 11 months, and may be extended to 2 years following viral injection.

### Example 12. Treatment of a Subject in Need Thereof with an ALS (In Vivo)

A subject with a ALS is treated with recombinant AAV obtained from the method of Example 2. The subject's neurological symptoms include impairment of a voluntary movement and speech changes, impairment of gait and posture, impairment of motor and sensory functions. Recombinant AAV is injected at a concentration range of 10¹⁰ particles/mL and 10¹⁴ particles/mL with a volume ranging from 10 μL to 1000 μL into the spinal cord or through intrathecal injection of a human subject with a neurological condition. The human subject's neurological condition symptoms and behavioral metric's are observed before, during, and post injection. Post injection observations are performed once a week until the first month post injection. After the first month post injection, observations are performed once a month for the next 11 months, and may be extended to 2 years following viral injection.

### Example 13. In Vitro Transgene Expression Induced by Isl1 Vectors

### Materials and Methods

### Cell Culture: Lec2 Cells

Lec2 cells (ATCC Cat #CRL-1736) are a mutant clone of epithelial cell line derived from CHO (Chinese Hamster Ovary) cell line. (Stanley P, Siminovitch L. Somatic Cell Genet. 3: 391-405, 1977. PubMed: 601679). Lec2 cells are maintained in 37° C. incubator with 5% CO₂ in media composed of αMEM supplemented with 10% FBS, 2.5 mM Glutamine, and penicillin/streptomycin. Cells are sub-cultured at a 1:5 ratio when reaching 90-100% confluency. Lec2 cells can be transfected and transduced at high efficiency. They are a good alternative to astrocytes for assessing the gene expression of the vectors.

Vectors: The vectors are tested via transfection of Lec2 cells:
NXL-P141 (CE-pGfa681-CRGI-hIsl1-oPRE-bGHpA)
NXL-P181 (CE-pGfa681-hIsl1-SpA)

Transfection and Immunofluorescence: Lec2 cells are seeded on glass cover slips in 24-well plates at 30-50% confluency 24 hours prior to transfection. Cells are transfected with 500 ng of plasmid DNA each using Lipofectamine reagent (Thermo Fisher Cat #15338) following manufacturer's protocol. At 24-48 hours post transfection, cells are fixed with 4% paraformaldehyde in PBS and subsequently washed and immunostained with anti-Isl1 (DSHB, 40.2D6) followed with secondary antibodies conjugated with fluorescent dyes (Invitrogen, Alexa Fluor). Images are captured under a fluorescent microscope (Zeiss Axiovert A1, Zen Blue). Gene expression levels are assessed by comparing the fluorescence intensity.

### In Vitro Studies Results:

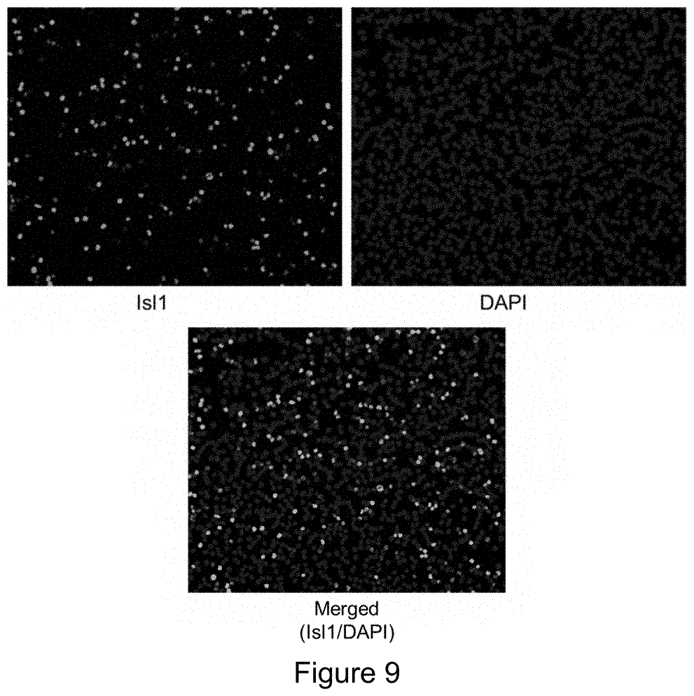

The tested hIs1 construct is effective in driving the expression of hIsl1 by transfection of the cultured cells, as demonstrated by the positive staining of hIsl1 in these cells (FIG. 9).

A variety of further modifications and improvements in and to the compositions and methods of the present disclosure will be apparent to those skilled in the art based. The following non-limiting embodiments are envisioned:
1. An adeno-associated virus (AAV) vector comprising a human insulin gene enhancer protein (hISL1) sequence comprising the nucleic acid sequence of SEQ ID NO: 6 and a human LIM-homeobox 3 (hLHX3) sequence comprising the nucleic acid sequence of SEQ ID NO: 13, where said hISL1 sequence and said hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, where said hISL1 sequence and said hLHX3 sequence are operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
2. An adeno-associated virus (AAV) vector comprising a nucleic acid coding sequence encoding a human insulin gene enhancer protein (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, wherein said hISL1 coding sequence and said hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 coding sequence and said hLHX3 coding sequence are operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
3. An adeno-associated virus (AAV) vector comprising an insulin gene enhancer protein (ISL1) nucleic acid coding sequence encoding a ISL1 protein and a LIM-homeobox 3 (LHX3) nucleic acid coding sequence encoding a LHX3 protein, wherein said ISL1 coding sequence and said LHX3 coding sequence are separated by a linker sequence, wherein said ISL1 coding sequence and said LHX3 coding sequence are operably linked to regulatory elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal sequence.
4. A composition comprising an adeno-associated virus (AAV) vector for converting glial cells to functional neurons in a human, wherein said AAV vector comprises a human insulin gene enhancer protein (hISL1) sequence having a nucleic acid sequence of SEQ ID NO: 6 and a human LIM-homeobox 3 (hLHX3) sequence having a nucleic acid sequence of SEQ ID NO: 13, wherein said hISL1 sequence and said hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 sequence and hLHX3 sequence are operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
5. A composition comprising an adeno-associated-virus (AAV) vector for converting glial cells to functional neurons in a human, wherein said AAV vector comprises a nucleic acid coding sequence encoding a human insulin gene enhancer protein (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, wherein said hISL1 coding sequence and said hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 coding sequence and said hLHX3 coding sequence are operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
6. A composition comprising an adeno-associated virus (AAV) vector for the treatment of a subject in need thereof, wherein said AAV vector comprises an insulin gene enhancer protein (ISL1) sequence and a LIM-homeobox 3 (LHX3) sequence, wherein said ISL1 sequence and said LHX3 sequence are separated by a linker sequence, wherein said ISL1 sequence and LHX3 sequence are operably linked to expression control elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal.
7. An adeno-associated virus (AAV) vector comprising a human insulin gene enhancer protein (hISL1) sequence comprising the nucleic acid sequence of SEQ ID NO: 6, wherein said hISL1 sequence is operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
8. An adeno-associated virus (AAV) vector comprising a nucleic acid coding sequence encoding a human insulin gene enhancer protein (hISL1) protein comprising the amino acid coding sequence of SEQ ID NO: 10, wherein said hISL1 coding sequence is operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
9. An adeno-associated virus (AAV) vector comprising an insulin gene enhancer protein (ISL1) nucleic acid coding sequence encoding a ISL1 protein, wherein said ISL1 coding sequence is operably linked to regulatory elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal sequence.
10. A composition comprising an adeno-associated virus (AAV) vector for converting glial cells to functional neurons in a human, wherein said AAV vector comprises a human insulin gene enhancer protein (hISL1) sequence having a nucleic acid sequence of SEQ ID NO: 6, wherein said hISL1 sequence is operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
11. A composition comprising an adeno-associated-virus (AAV) vector for converting glial cells to functional neurons in a human, wherein said AAV vector comprises a nucleic acid coding sequence encoding a human insulin gene enhancer protein (hISL1) protein comprising the amino acid sequence of SEQ ID NO: 10, wherein said hISL1 coding sequence is operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
12. A composition comprising an adeno-associated virus (AAV) vector for the treatment of a subject in need thereof, wherein said AAV vector comprises an insulin gene enhancer protein (ISL1) sequence, wherein said ISL1 sequence is operably linked to expression control elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal.
13. An adeno-associated virus (AAV) vector comprising a human LIM-homeobox 3 (hLHX3) sequence comprising the nucleic acid sequence of SEQ ID NO: 13, where said hLHX3 sequence are operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
14. An adeno-associated virus (AAV) vector comprising a nucleic acid coding sequence encoding a human LIM-homeobox 3 (hLHX3) protein comprising the amino acid sequence of SEQ ID NO: 14, wherein said hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 coding sequence and said hLHX3 coding sequence are operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
15. An adeno-associated virus (AAV) vector comprising a LIM-homeobox 3 (LHX3) nucleic acid coding sequence encoding a LHX3 protein, wherein said LHX3 coding sequence is operably linked to regulatory elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal sequence.
16. A composition comprising an adeno-associated virus (AAV) vector for converting glial cells to functional neurons in a human, wherein said AAV vector comprises a human LIM-homeobox 3 (hLHX3) sequence having a nucleic acid sequence of SEQ ID NO: 13, wherein said hLHX3 sequence are operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
17. A composition comprising an adeno-associated-virus (AAV) vector for converting glial cells to functional neurons in a human, wherein said AAV vector comprises a nucleic acid coding sequence encoding a human LIM-homeobox 3 (hLHX3) protein comprising the amino acid sequence of SEQ ID NO: 14, wherein said hLHX3 coding sequence are operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
18. A composition comprising an adeno-associated virus (AAV) vector for the treatment of a subject in need thereof, wherein said AAV vector comprises a LIM-homeobox 3 (LHX3) sequence, wherein said LHX3 sequence are operably linked to expression control elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal.
19. The AAV vector of any one of embodiments 1-3, 7-8, or 13-15, or the composition of any one of embodiments 4-6, 10-12, and 16-18, wherein said AAV vector is selected from the group consisting of AAV serotype 2, AAV serotype 5, and AAV serotype 9.
20. The AAV vector or composition of embodiment 19, wherein said AAV vector is AAV serotype 2.
21. The AAV vector or composition of embodiment 19, wherein said AAV vector is AAV serotype 5.
22. The AAV vector or composition of embodiment 19, wherein said AAV vector is AAV serotype 9.
23. The composition of embodiment 4 5, 10, 11, 16, or 17, wherein said glial cells are reactive astrocytes.
24. The composition of embodiment 4 5, 10, 11, 16, or 17, wherein said functional neurons are selected from the group consisting of glutamatergic neurons, GABAergic neurons, dopaminergic neurons, cholinergic neurons, seratonergic neurons, epinephrinergic neurons, motor neurons, and peptidergic neurons.
25. The composition of embodiment 4 5, 10, 11, 16, or 17 wherein said human has a neurological condition.
26. The AAV vector of embodiment 3 or 9, or the composition of embodiment 6 or 12, wherein said ISL1 is a human ISL1 (hISL1).
27. The AAV vector of embodiment 3 or 15, or the composition of embodiment 6 or 18, wherein said LHX3 is a human LHX3 (hLHX3).
28. The AAV vector of embodiment 3 or 9, or the composition of embodiment 6 or 12, wherein said ISL1 is selected from the group consisting of a chimpanzee ISL1, a bonobo ISL1, an orangutan ISL1, a gorilla ISL1, a macaque ISL1, a marmoset ISL1, a capuchin ISL1, a baboon ISL1, a gibbon ISL1, and a lemur ISL1.
29. The AAV vector of embodiment 3 or 15, or the composition of embodiment 6 or 18, wherein said LHX3 is selected from the group consisting of a chimpanzee LHX3, a bonobo LHX3, an orangutan LHX3, a gorilla LHX3, a macaque LHX3, a marmoset LHX3, a capuchin LHX3, a baboon LHX3, a gibbon LHX3, and a lemur LHX3.
30. The AAV vector or composition of embodiment 26, wherein said hISL1 comprises a nucleic acid sequence encoding an amino acid sequence at least 80% identical or similar to SEQ ID NO: 10.
31. The AAV vector or composition of embodiment 27, wherein said hLHX3 comprises a nucleic acid sequence encoding an amino acid sequence at least 80% identical or similar to SEQ ID NO: 14.
32. The AAV vector or composition of embodiment 26, wherein said hISL1 coding sequence comprises a nucleic acid sequence at least 80% identical to SEQ ID NO: 6, or the complement thereof.
33. The AAV vector or composition of embodiment 27, said hLHX3 coding sequence comprises a nucleic acid sequence at least 80% identical to SEQ ID NO: 13, or the complement thereof.
34. The AAV vector of embodiment 3, or the composition of embodiment 6, wherein said linker is selected from the group consisting of P2A and T2A.
35. The AAV vector or composition of embodiment 34, wherein said linker is said P2A.
36. The AAV vector or composition of embodiment 34, wherein said linker is said T2A
37. The AAV vector or composition of embodiment 34, wherein said P2A linker comprises a nucleic acid sequence at least 80% identical to the sequence selected from the group consisting of SEQ ID NO: 15 and 18, or the complement thereof.
38. The AAV vector or composition of embodiment 34, wherein said T2A linker comprises a nucleic acid sequence at least 80% identical to the sequence selected from the group consisting of SEQ ID NO: 16 and 19, or the complement thereof.
39. The AAV vector of embodiment 3, 9, or 10, or the composition of embodiment 6, 12, or 18, wherein said GFAP promoter is a human GFAP (hGFAP) promoter.
40. The AAV vector of embodiment 3, 9, or 10, or the composition of embodiment 6, 12, or 18, wherein said GFAP promoter is selected from the group consisting of a chimpanzee GFAP promoter, a bonobo GFAP promoter, an orangutan GFAP promoter, a gorilla GFAP promoter, a macaque GFAP promoter, a marmoset GFAP promoter, a capuchin GFAP promoter, a baboon GFAP promoter, a gibbon GFAP promoter, and a lemur GFAP promoter.
41. The AAV vector or composition of embodiment 39, wherein said hGFAP promoter comprises a nucleic acid sequence at least 80% identical to SEQ ID NO: 3, or the complement thereof.
42. The AAV vector or composition of embodiment 39, wherein said hGFAP promoter comprises a nucleic acid sequence at least 80% identical to SEQ ID NOs: 4 or the complement thereof.
43. The AAV vector or composition of embodiment 39, wherein said hGFAP promoter comprises a nucleic acid sequence at least 80% identical to SEQ ID NOs: 12 or the complement thereof.
44. The AAV vector of embodiment 3, 9, or 10, or the composition of embodiment 6, 12, or 18, wherein said enhancer is selected from the group consisting of an enhancer from human elongation factor-1 alpha (EF1-α) promoter and cytomegalovirus (CMV) enhancer
45. The AAV vector or composition of embodiment 44, wherein said EF1-α comprises a nucleic acid sequence at least 80% identical to SEQ ID NO: 2, or the complement thereof.
46. The AAV vector or composition of embodiment 44, wherein said CMV enhancer comprises a nucleic acid sequence at least 80% identical to SEQ ID NO: 11, or the complement thereof.
47. The AAV vector of embodiment 3, 9, or 10, or the composition of embodiment 6, 12, or 18, wherein said chimeric intron comprises a nucleic acid sequence at least 80% identical to a nucleic acid sequence selected from the group consisting of SEQ ID NO: 5 and 22, or the complement thereof.
48. The AAV vector of embodiment 3, 9, or 10, or the composition of embodiment 6, 12, or 18, wherein said WPRE comprises a nucleic acid sequence at least 80% identical to a nucleic acid sequence selected from the group consisting of SEQ ID NO: 7 and 23, or the complement thereof.
49. The AAV vector of embodiment 3, 9, or 10, or the composition of embodiment 6, 12, or 18, wherein said polyadenylated signal is selected from the group consisting of SV40 polyadenylation signal, a hGH polyadenylation signal, a bGH polyadenylation signal, and a synthetic polyadenylation signal.
50. The AAV vector or composition of embodiment 49, wherein said SV40 polyadenylated signal comprises a nucleic acid sequence at least 80% identical to SEQ ID NO: 8, or the complement thereof.
51. The AAV vector or composition of embodiment 49, wherein said hGH polyadenylated signal comprises a nucleic acid sequence at least 80% identical to SEQ ID NO: 17, or the complement thereof.
52. The AAV vector of embodiment 3, 9, or 15, or the composition of embodiment 6, 12, or 18, wherein said AAV vector further comprises a nucleic acid sequence encoding an AAV protein sequence.
53. The AAV vector of any one of embodiments 1-3, 7-9, or 13-15, or the composition of any one of embodiments 4-6. 10-12, 16-18, wherein said AAV vector comprises AAV serotype 2 inverted terminal repeats (ITRs).
54. The AAV vector of any one of embodiments 1-3, 7-9, or 13-15, or the composition of any one of embodiments 4-6. 10-12, 16-18, wherein said AAV vector comprises AAV serotype 5 inverted terminal repeats (ITRs).
55. The AAV vector of any one of embodiments 1-3, 7-9, or 13-15, or the composition of any one of embodiments 4-6. 10-12, 16-18, wherein said AAV vector comprises AAV serotype 9 inverted terminal repeats (ITRs).
56. The AAV vector of any one of embodiments 1-3, 7-9, or 13-15, or the composition of any one of embodiments 4-6. 10-12, 16-18, wherein said AAV vector comprises at least one ITR nucleic acid sequence at least 80% identical to SEQ ID NO: 1.
57. The AAV vector of any one of embodiments 1-3, 7-9, or 13-15, or the composition of any one of embodiments 4-6. 10-12, 16-18, wherein said AAV vector comprises at least one ITR nucleic acid sequence at least 80% identical to SEQ ID NO: 9.
58. The composition of embodiment 6, 12, or 18, wherein said subject in need thereof is a mammal.
59. The composition of embodiment 58, wherein said mammal is a human.
60. The composition of embodiment 58, wherein said mammal is a non-human primate.
61. The composition of embodiment 6, 12, or 18, wherein said subject in need thereof has a neurological condition.
62. The composition of embodiment 25 or 61, wherein said neurological condition comprises an injury to the central nervous system (CNS) or peripheral nervous system.
63. The composition of embodiment 25 or 61, wherein said wherein said neurological condition comprises an injury to the CNS.
64. The composition of embodiment 25 or 61, wherein said neurological condition is selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.
65. The composition of embodiment 25 or 61, wherein said neurological condition is Alzheimer's Disease.
66. The composition of embodiment 25 or 61, wherein said neurological condition is Parkinson's Disease.
67. The composition of embodiment 25 or 61, wherein said neurological condition is ALS.
68. The composition of embodiment 25 or 61, wherein said neurological condition is Huntington's Disease.
69. The composition of embodiment 25 or 61, wherein said neurological condition is a stroke.
70. The composition of embodiment 69, wherein said stroke is an ischemic stroke.
71. The composition of embodiment 69, wherein said stroke is a hemorrhagic stroke.
72. The composition of embodiment 61, wherein said composition is capable of converting at least one glial cell to a neuron.
73. The composition of embodiment 72, wherein said glial cells are selected from the group consisting of astrocytes and NG2 cells.
74. The composition of embodiment 72, wherein said glial cells are astrocytes.
75. The composition of embodiment 74, wherein said astrocytes are reactive astrocytes.
76. The composition of embodiment 72, wherein said glial cells are GFAP positive.
77. The composition of embodiment 72, wherein said neurons are functional neurons.
78. The composition of embodiment 72, wherein said functional neurons are selected from the group consisting of glutamatergic neurons, GABAergic neurons. dopaminergic neurons, cholinergic neurons, seratonergic neurons, epinephrinergic neurons, motor neurons, and peptidergic neurons.
79. The composition of embodiment 78, wherein said functional neurons are glutamatergic neurons.
80. The composition of embodiment 6, wherein said composition is formulated to be delivered to a subject in need thereof.
81. The composition of embodiment 80, wherein said composition is formulated for local delivery.
82. The composition of embodiment 80, wherein said composition is formulated for systemic delivery.
83. The composition of any one of embodiments 80-82, wherein said composition is formulated for delivery via intraperitoneal, intramuscular, intravenous, intrathecal, intracerebral, intracranial, intra lateral ventricle of the brain, intra cisterna magna, intra vitreous, intra-subretina, intraparenchymal, intranasal, or oral administration.
84. A method comprising delivering the composition of embodiment 6 to said subject in need thereof.
85. The method of embodiment 84, wherein said composition is formulated to be delivered to a subject in need thereof.
86. The method of embodiment 84, wherein said delivering comprises local administration.
87. The method of embodiment 84, wherein said delivering comprises systemic administration.
88. The method of any one of embodiments 84-87, wherein said delivering comprises an intraperitoneal, intramuscular, intravenous, intrathecal, intracerebral, intracranial, intra lateral ventricle of the brain, intra cisterna magna, intra vitreous, intra-subretina, intraparenchymal, intranasal, or oral administration.
89. A method of converting reactive astrocytes to functional neurons in a spinal cord of a living human comprising: injecting an adeno-associated virus (AAV) into a subject in need thereof, wherein said AAV comprises a DNA vector construct comprising a human insulin gene enhancer protein (hISL1) sequence comprising the nucleic acid sequence of SEQ ID NO: 6 and a human LIM-homeobox 3 (hLHX3) sequence comprising the nucleic acid sequence of SEQ ID NO: 13, wherein said hISL1 sequence and said hLHX3 sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 sequence and said hLHX3 sequence are operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
90. A method of converting reactive astrocytes to functional neurons in a spinal cord of a living human comprising: injecting an adeno-associated virus (AAV) into a subject in need thereof, wherein said AAV comprises a DNA vector construct comprising a nucleic acid coding sequence encoding a human insulin gene enhancer protein (hISL1) protein comprising the amino acid sequence of SEQ ID NO: 10 and a nucleic acid coding sequence encoding a human LIM-homeobox 3 (hLHX3) protein comprising the amino acid sequence of SEQ ID NO: 14, wherein said hISL1 coding sequence and said hLHX3 coding sequence are separated by a P2A linker comprising the nucleic acid selected from the group consisting of SEQ ID NO: 15 and 18 or a T2A linker comprising the nucleic acid sequence selected from the group consisting of SEQ ID NO: 16 and 19, wherein said hISL1 coding sequence and hLHX3 coding sequence are operably linked to expression control elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
91. A method of converting glial cells to neurons in a subject in need thereof comprising: delivering an adeno-associated virus (AAV) to said subject in need thereof, wherein said AAV comprises a DNA vector construct comprising an insulin gene enhancer protein (ISL1) sequence and a LIM-homeobox 3 (LHX3) sequence, wherein said ISL1 sequence and LHX3 sequence are separated by a linker sequence, wherein said ISL1 sequence and LHX3 sequence are operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) and a polyadenylation signal sequence, wherein said vector is capable of converting at least one glial cell to a neuron in said subject in need thereof.
92. A method of treating a neurological condition in a subject in need thereof comprising: delivering an adeno-associated virus (AAV) to said subject, wherein said AAV comprises a DNA vector construct comprising an insulin gene enhancer protein (ISL1) sequence and LIM-homeobox 3 (LHX3) sequence, wherein said ISL1 sequence and LHX3 sequence are separated by a linker sequence, wherein said ISL1 sequence and said LHX3 sequence are operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) a polyadenylation signal to said subject in need thereof.
93. A method of converting reactive astrocytes to functional neurons in a spinal cord of a living human comprising: injecting an adeno-associated virus (AAV) into a subject in need thereof, wherein said AAV comprises a DNA vector construct comprising a human insulin gene enhancer protein (hISL1) sequence comprising the nucleic acid sequence of SEQ ID NO: 6, wherein said hISL1 sequence is operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22; (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
94. A method of converting reactive astrocytes to functional neurons in a spinal cord of a living human comprising: injecting an adeno-associated virus (AAV) into a subject in need thereof, wherein said AAV comprises a DNA vector construct comprising a nucleic acid coding sequence encoding a human insulin gene enhancer protein (hISL1) protein comprising the amino acid sequence of SEQ ID NO: 10. wherein said hISL1 coding sequence is operably linked to expression control elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
95. A method of converting glial cells to neurons in a subject in need thereof comprising: delivering an adeno-associated virus (AAV) to said subject in need thereof, wherein said AAV comprises a DNA vector construct comprising an insulin gene enhancer protein (ISL1) sequence, wherein said ISL1 sequence is operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) and a polyadenylation signal sequence, wherein said vector is capable of converting at least one glial cell to a neuron in said subject in need thereof.
96. A method of treating a neurological condition in a subject in need thereof comprising: delivering an adeno-associated virus (AAV) to said subject, wherein said AAV comprises a DNA vector construct comprising an insulin gene enhancer protein (ISL1) sequence, wherein said ISL1 sequence is operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) a polyadenylation signal to said subject in need thereof.
97. A method of converting reactive astrocytes to functional neurons in a spinal cord of a living human comprising: injecting an adeno-associated virus (AAV) into a subject in need thereof, wherein said AAV comprises a DNA vector construct comprising a human LIM-homeobox 3 (hLHX3) sequence comprising the nucleic acid sequence of SEQ ID NO: 13, wherein said hLHX3 sequence is operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
98. A method of converting reactive astrocytes to functional neurons in a spinal cord of a living human comprising: injecting an adeno-associated virus (AAV) into a subject in need thereof, wherein said AAV comprises a DNA vector construct comprising a nucleic acid coding sequence encoding a human LIM-homeobox 3 (hLHX3) protein comprising the amino acid sequence of SEQ ID NO: 14, wherein said hLHX3 coding sequence is operably linked to expression control elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence of SEQ ID NO: 5 or 22;
   (d) a woodchuck hepatitis virus posttranscriptional regulatory element (WPRE) comprising the nucleic acid sequence of SEQ ID NO: 7 or 23; and
   (e) a polyadenylation signal comprising a nucleic acid sequence selected from the group consisting of SEQ ID NOs: 8, 17, 24, and 25.
99. A method of converting glial cells to neurons in a subject in need thereof comprising: delivering an adeno-associated virus (AAV) to said subject in need thereof, wherein said AAV comprises a DNA vector construct comprising a LIM-homeobox 3 (LHX3) sequence, wherein said LHX3 sequence is operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) and a polyadenylation signal sequence, wherein said vector is capable of converting at least one glial cell to a neuron in said subject in need thereof.
100. A method of treating a neurological condition in a subject in need thereof comprising: delivering an adeno-associated virus (AAV) to said subject, wherein said AAV comprises a DNA vector construct comprising a LIM-homeobox 3 (LHX3) sequence, wherein said LHX3 sequence is operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) a polyadenylation signal to said subject in need thereof.
101. The method of any one of embodiments 89-92, wherein said AAV is selected from the group consisting of AAV serotype 2, AAV serotype 5, and AAV serotype 9.
102. The method of embodiment 101, wherein said AAV is AAV serotype 2.
103. The method of embodiment 101, wherein said AAV is AAV serotype 5.
104. The method of embodiment 101, wherein said AAV is AAV serotype 9.
105. The method of embodiments 89 90, 93, 94, 97, or 98, wherein said functional neurons are glutamatergic neurons, GABAergic neurons, dopaminergic neurons, cholinergic neurons, seratonergic neurons, epinephrinergic neurons, motor neurons, and peptidergic neurons.
106. The method of embodiments 91 92, 95 or 96, wherein said ISL1 is human ISL1 (hISL1).
107. The method of embodiments 91, 92, 99, or 100, wherein said LHX3 is human LHX3 (hLHX3).
108. The method of embodiments 91, 92, 95, or 96, wherein said ISL1 is selected from the group consisting of a chimpanzee ISL1, a bonobo ISL1, an orangutan ISL1, a gorilla ISL1, a macaque ISL1, a marmoset ISL1, a capuchin ISL1, a baboon ISL1, a gibbon ISL1, and a lemur ISL1.
109. The method of embodiments 91, 92, 99, or 100, wherein said LHX3 is selected from the group consisting of a chimpanzee LHX3, a bonobo LHX3, an orangutan LHX3, a gorilla LHX3, a macaque LHX3, a marmoset LHX3, a capuchin LHX3, a baboon LHX3, a gibbon LHX3, and a lemur LHX3.
110. The method of embodiment 106, wherein said hISL1 comprises an amino acid sequence encoding an amino acid coding sequence at least 80% identical or similar to SEQ ID NO: 10.
111. The method of embodiment 107, said hLHX3 comprises a amino acid sequence encoding an amino acid sequence at least 80% identical or similar to SEQ ID NO: 14.
112. The method of embodiment 106, wherein said hISL1 coding sequence comprises a nucleic acid sequence at least 80% identical to SEQ ID NO: 6, or the complement thereof.
113. The method of embodiment 107, said hLHX3 coding sequence comprises a nucleic acid sequence at least 80% identical to SEQ ID NO: 13, or the complement thereof.
114. The method of embodiments 91, 92, 95, 96, 99, or 100, wherein said GFAP promoter is a human GFAP (hGFAP) promoter.
115. The method of embodiments 91 92, 95, 96, 99, or 100, wherein said GFAP promoter is selected from the group consisting of a chimpanzee GFAP promoter, a bonobo GFAP promoter, an orangutan GFAP promoter, a gorilla GFAP promoter, a macaque GFAP promoter, a marmoset GFAP promoter, a capuchin GFAP promoter, a baboon GFAP promoter, a gibbon GFAP promoter, and a lemur GFAP promoter.
116. The method of embodiment 114, wherein said hGFAP promoter comprises a nucleic acid sequence at least 80% identical to SEQ ID NO: 3, or the complement thereof.
117. The method of embodiment 114, wherein said hGFAP promoter comprises a nucleic acid sequence at least 80% identical to SEQ ID NOs: 4, or the complement thereof.
118. The method of embodiment 114, wherein said hGFAP promoter comprises a nucleic acid sequence at least 80% identical to SEQ ID NOs: 12, or the complement thereof.
119. The method of embodiments 91 or 92, wherein said linker is selected from the group consisting of P2A and T2A.
120. The method of embodiment 119, wherein said P2A linker comprises a nucleic acid sequence at least 80% identical to the sequence selected from the group consisting of SEQ ID NO: 15 and 18, or the complement thereof.
121. The method of embodiment 119, wherein said T2A linker comprises a nucleic acid sequence at least 80% identical the sequence selected from the group consisting of SEQ ID NO: 16 and 19, or the complement thereof.
122. The method of embodiments 91 92, 95, 96, 99, or 100, wherein said enhancer is selected from the group consisting of an enhancer from human elongation factor-1 alpha (EF1-α) promoter and cytomegalovirus (CMV) enhancer.
123. The method of embodiment 122, wherein said EF1-α comprises a nucleic acid sequence at least 80% identical to SEQ ID NO: 2, or the complement thereof.
124. The method of embodiment 122, wherein said CMV enhancer comprises a nucleic acid sequence at least 80% identical to SEQ ID NO: 11, or the complement thereof.
125. The method of embodiments 91 92, 95, 96, 99, or 100, wherein said chimeric intron comprises a nucleic acid sequence at least 80% identical to a nucleic acid sequence selected from the group consisting of SEQ ID NO: 5 and 22, or the complement thereof.
126. The method of embodiments 91, 92, 95, 96, 99, or 100, wherein said WPRE comprises a nucleic acid sequence at least 80% identical to a nucleic acid sequence selected from the group consisting of SEQ ID NO: 7 and 23, or the complement thereof.
127. The method of embodiments 91 92, 95, 96, 99, or 100, wherein said polyadenylated signal comprises a nucleic acid sequence selected from the group consisting of a SV40 polyadenylation signal, a hGH polyadenylation signal, a bGH polyadenylation signal, and a synthetic polyadenylation signal.
128. The method of embodiment 127, wherein said SV40 polyadenylated signal comprises a nucleic acid sequence at least 80% identical to SEQ ID NO: 8, or the complement thereof.
129. The method of embodiment 127, wherein said hGH polyadenylated signal comprises a nucleic acid sequence at least 80% identical to SEQ ID NO: 17, or the complement thereof.
130. The method of embodiments 91, 92, 95, 96, 99, or 100, wherein said vector further comprises a nucleic acid sequence encoding an AAV protein sequence.
131. The method of any one of embodiments 89-92, wherein said vector comprises AAV serotype 2 inverted terminal repeats (ITRs).
132. The method of any one of embodiments 89-92, wherein said vector comprises AAV serotype 5 inverted terminal repeats (ITRs).
133. The method of any one of embodiments 89-92, wherein said AAV comprises AAV serotype 9 inverted terminal repeats (ITRs).
134. The method of any one of embodiments 89-92, wherein said AAV comprises at least one ITR nucleic acid sequence at least 80% identical to SEQ ID NO: 1.
135. The method of any one of embodiments 89-92, wherein said AAV comprises at least one ITR nucleic acid sequence at least 80% identical to SEQ ID NO: 9.
136. The method of embodiment 91, 95, or 99. wherein said converting occurs in the central nervous system (CNS) or peripheral nervous system.
137. The method of embodiment 91, 95, or 99, wherein said converting occurs in the CNS.
138. The method of embodiment 91, 92, 95, 96, 99, or 100, wherein said subject in need thereof is a mammal.
139. The method of embodiment 138, wherein said mammal is a human.
140. The method of embodiment 138, wherein said mammal is a non-human primate.
141. The method of embodiment 91, 92, 95, 96, 99, or 100, wherein said delivering comprises a local administration.
142. The method of embodiment 91, 92, 95, 96, 99, or 100, wherein said delivering comprises systemic administration.
143. The method of embodiment 91, 92, 95, 96, 99, or 100, said delivering comprises an administration selected from the group consisting of an intraperitoneal administration, intramuscular administration, intravenous administration, intrathecal administration, intracerebral administration, intracranial, intra lateral ventricle of the brain, intra cisterna magna, intra vitreous, intra-subretina, intraparenchymal administration, intranasal administration, and oral administration.
144. The method of embodiment 89, 90, 93, 94, 97, or 98, wherein said injecting comprises an injection selected from the group consisting of an intraperitoneal injection, intramuscular injection, intravenous injection, intrathecal injection, intracerebral injection, intracranial, intra lateral ventricle of the brain, intra cisterna magna, intra vitreous, intra-subretina, intraparenchymal injection, intranasal injection, and oral injection.
145. The method of embodiments 91, 92, 95, 96, 99, or 100, wherein said delivering comprises injecting.
146. The method of any one of embodiments 89, 90, 93, 94, 97, 98, or 145, wherein said injecting is performed at a concentration of between 10¹⁰ particles/mL and 10¹⁴ particles/mL.
147. The method of embodiment 146, wherein said injecting further comprises a flow rate of between 0.1 μL/minute and 5.0 μL/minute.
148. The method of embodiment 91, 95, or 99, wherein said at least one glial cell is selected from the group consisting of at least one astrocyte and at least one NG2 cell.
149. The method of embodiment 148, wherein said at least one glial cell is at least one astrocyte.
150. The method of embodiment 148 or 149, wherein said at least one astrocyte is a reactive astrocyte.
151. The method of embodiment 91, 95, or 99, wherein said neuron is a functional neuron.
152. The method of any one of embodiments 89, 90, 93, 94, 97, 98, or 151, wherein said functional neurons are selected from the group consisting of glutamatergic neurons, GABAergic neurons, dopaminergic neurons, cholinergic neurons, seratonergic neurons, epinephrinergic neurons, motor neurons, and peptidergic neurons.
153. The method of embodiment 91, 95, or 99, wherein said subject exhibits an improvement of at least one neurological condition symptom as compared to said subject prior to said delivering.
154. The method of embodiment 153, wherein said improvement is measured within 1 year of said delivering.
155. The method of any one of embodiments 89, 90, 93, 94, 97, 98, or 145, wherein said method comprises directly injecting said AAV into the brain of said subject.
156. The method of any one of embodiments 89, 90, 93, 94, 97, 98, or 145, wherein said method comprises directly injecting said AAV into the striatum of said subject.
157. The method of any one of embodiments 89, 90, 93, 94, 97, 98, or 145, wherein said method comprises directly injecting said AAV into the spinal cord of said subject.
158. The method of embodiments 92, 96, or 100, wherein said neurological condition comprises an injury to the central nervous system (CNS) or peripheral nervous system.
159. The method of embodiments 92, 96, or 100, wherein said neurological condition is selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.
160. The method of embodiments 92, 96, or 100, wherein said neurological condition is Alzheimer's Disease.
161. The method of embodiments 92, 96, or 100, wherein said neurological condition is Parkinson's Disease.
162. The method of embodiments 92, 96, or 100, wherein said neurological condition is ALS.
163. The method of embodiments 92, 96, or 100, wherein said neurological condition is Huntington's Disease.
164. The method of embodiments 92, 96, or 100, wherein said neurological condition is a stroke.
165. The method of embodiment 164, wherein said stroke is an ischemic stroke.
166. The method of embodiment 164, wherein said stroke is a hemorrhagic stroke.
167. The method of embodiments 92, 96, or 100, wherein said method is capable of converting at least one glial cell into a neuron.
168. The method of embodiment 167, wherein said glial cells are selected from the group consisting of astrocytes and NG2 cells.
169. The method of embodiment 167, wherein said glial cells are astrocytes.
170. The method of embodiment 169, wherein said astrocytes are reactive astrocytes.
171. The method of embodiment 167, wherein said glial cells are GFAP positive.
172. The method of embodiment 167, wherein said neurons are functional neurons.
173. The method of embodiment 172, wherein said functional neurons are selected from the group consisting of glutamatergic neurons, GABAergic neurons, dopaminergic neurons, cholinergic neurons, seratonergic neurons, epinephrinergic neurons, motor neurons, and peptidergic neurons.
174. The method of embodiments 89, 90, 93, 94, 97, 98, wherein a therapeutically effective dose of said AAV is injected into said subject.
175. The method of embodiments 91, 92, 93, 94, 97, 98, wherein a therapeutically effective dose of said AAV is delivered to said subject.
176. The method of embodiment 174 or 175, wherein said therapeutically effective dose is administered with a pharmaceutically acceptable carrier.
177. The AAV vector or composition of embodiment 49, wherein said bGH polyadenylated signal comprises a nucleic acid sequence at least 80% identical to SEQ ID NO: 24, or the complement thereof.
178. The AAV vector or composition of embodiment 49, wherein said synthetic polyadenylated signal comprises a nucleic acid sequence at least 80% identical to SEQ ID NO: 25, or the complement thereof.
179. The method of embodiment 127, wherein said bGH polyadenylated signal comprises a nucleic acid sequence at least 80% identical to SEQ ID NO: 24, or the complement thereof.
180. The method of embodiment 127, wherein said synthetic polyadenylated signal comprises a nucleic acid sequence at least 80% identical to SEQ ID NO: 25, or the complement thereof.

1. An adeno-associated virus (AAV) vector comprising
a nucleic acid sequence encoding a human insulin gene enhancer protein (hISL1) sequence, wherein the nucleic acid sequence encodes an amino acid sequence of SEQ ID NO: 10 or a portion thereof, or wherein the nucleic acid sequence comprises a nucleic acid sequence of SEQ ID NO: 6 or a portion thereof; and
a nucleic acid sequence encoding a human LIM-homeobox 3 (hLHX3) sequence, wherein the nucleic acid sequence encodes an amino acid sequence of SEQ ID NO: 14 or a portion thereof, or wherein the nucleic acid sequence comprises a nucleic acid sequence of SEQ ID NO: 13,
where said hISL1 sequence and said hLHX3 sequence are separated by a (i)_P2A linker comprising a nucleic acid selected from the group consisting of SEQ ID NO: 15 and 18, or (ii) a T2A linker comprising a nucleic acid sequence selected from the group consisting of SEQ ID NO: 16 and 19, where said hISL1 sequence and said hLHX3 sequence are operably linked to regulatory elements comprising:
(a) a glial fibrillary acidic protein (GFAP) promoter;
(b) an enhancer from a human elongation factor-1 alpha (EF1-α) promoter or a cytomegalovirus (CMV) enhancer;
(c) a chimeric intron;
(d) a woodchuck hepatitis virus posttranscriptional regulatory element (WPRE); and
(e) a polyadenylation signal.

2. The AAV vector of claim 1, wherein:
(a) the glial fibrillary acidic protein (GFAP) promoter comprises a nucleic acid sequence at least 80% identical to a sequence selected from the group consisting of SEQ ID NOs: 3, 4, and 12;
(b) the enhancer from a human elongation factor-1 alpha (EF1-α) promoter comprises a nucleic acid sequence at least 80% identical to SEQ ID NO: 2 or the cytomegalovirus (CMV) enhancer comprises a nucleic acid sequence at least 80% identical to SEQ ID NO: 11;
(c) the chimeric intron comprises a nucleic acid sequence at least 80% identical to SEQ ID NO: 5 or 22;
(d) the woodchuck hepatitis virus posttranscriptional regulatory element (WPRE) comprises a nucleic acid sequence at least 80% identical to SEQ ID NO: 7 or 23; or
(e) the polyadenylation signal comprises a nucleic acid sequence a least 80% identical to a sequence selected from the group consisting of SEQ ID NOS: 8, 17, 24, and 25.

3. (canceled)

4. A composition comprising one or more adeno-associated virus (AAV) vectors for converting glial cells to functional neurons in a subject, wherein said one or more AAV vectors comprises
(i) a nucleic acid sequence encoding a human insulin gene enhancer protein (hISL1) sequence, wherein the nucleic acid sequence encodes an amino acid sequence of SEQ ID NO: 10 or a portion thereof, or wherein the nucleic acid sequence comprises a nucleic acid sequence of SEQ ID NO: 6 or a portion thereof; and/or
(ii) a nucleic acid sequence encoding a human LIM-homeobox 3 (hLHX3) sequence, wherein the nucleic acid sequence encodes an amino acid sequence of SEQ ID NO: 14 or a portion thereof, or wherein the nucleic acid sequence comprises a nucleic acid sequence of SEQ ID NO: 13 or a portion thereof,
wherein said hISL1 sequence and/or said hLHX3 sequence are operably linked to regulatory elements comprising:
(a) a human glial fibrillary acidic protein (GFAP) promoter;
(b) an enhancer from the human elongation factor-1 alpha (EF-1 alpha) promoter or a cytomegalovirus (CMV) enhancer;
(c) a chimeric intron;
(d) a woodchuck hepatitis virus posttranscriptional regulatory element (WPRE); and
(e) a polyadenylation signal.

5. The composition of claim 4, wherein:
(a) the human glial fibrillary acidic protein (GFAP) promoter comprises a nucleic acid sequence at least 80% identical to a sequence selected from the group consisting of SEQ ID NOs: 3, 4, and 12;
(b) the enhancer from the human elongation factor-1 alpha (EF-1 alpha) promoter comprises a nucleic acid sequence at least 80% identical to SEQ ID NO: 2 or the cytomegalovirus (CMV) enhancer comprises a nucleic acid sequence at least 80% identical to SEQ ID NO: 11;
(c) the chimeric intron comprises a nucleic acid sequence at least 80% identical to SEQ ID NO: 5 or 22;
(d) the woodchuck hepatitis virus posttranscriptional regulatory element (WPRE) comprises a nucleic acid sequence at least 80% identical to SEQ ID NO: 7 or 23; or
(e) the polyadenylation signal comprises a nucleic acid sequence at least 80% identical to a sequence selected from the group consisting of SEQ ID NOs: 8, 17, 24, and 25.

6.-18. (canceled)

19. The AAV vector of claim 1, wherein said AAV vector is selected from the group consisting of AAV serotype 2, AAV serotype 5, and AAV serotype 9.

20.-29. (canceled)

30. The AAV vector of claim 1, wherein said hISL1 sequence comprises an amino acid sequence at least 80% identical or similar to SEQ ID NO: 10; and/or wherein said hLHX3 sequence comprises an amino acid sequence at least 80% identical or similar to SEQ ID NO: 14.

31. (canceled)

32. The AAV vector of claim 1, wherein said nucleic acid sequence encoding the hISL1 sequence is at least 80% identical to SEQ ID NO: 6, or the complement thereof; and/or wherein said nucleic acid sequencing encoding the hLH3 sequence is at least 80% identical to SEQ ID NO: 13, or a complement thereof.

33. (canceled)

34. The composition of claim 4, wherein the nucleic acid sequence encoding the hISL1 sequence and the nucleic acid sequence encoding the hLHX3 sequence are present within one AAV vector and separated by a linker.

35. (canceled)

36. (canceled)

37. The composition of claim 34, wherein said linker is a P2A linker comprising a nucleic acid sequence at least 80% identical to a sequence selected from the group consisting of SEQ ID NO: 15 and 18, or a complement thereof.

38. The composition of claim 34, wherein said linker is a T2A linker comprising a nucleic acid sequence at least 80% identical to the sequence selected from the group consisting of SEQ ID NO: 16 and 19, or a complement thereof.

39.-51. (canceled)

52. The composition of claim 4, wherein at least one of said one or more AAV vectors is selected from the group consisting of AAV serotype 2, AAV serotype 5, and AAV serotype 9.

53-55. (canceled)

56. The AAV vector of claim 1, wherein said AAV vector comprises at least one ITR nucleic acid sequence at least 80% identical to SEQ ID NO: 1 or SEQ ID NO: 9.

57.-63. (canceled)

64. The composition of claim 4, wherein said subject has a neurological condition selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.

65.-90. (canceled)

91. A method of (i) converting at least one glial cell to a neuron in a subject in need thereof, (ii) treating a neurological condition in a subject in need thereof, or (iii) converting at least one reactive astrocyte to a functional neuron in a subject in need thereof, the method comprising: delivering a composition to said subject in need thereof, wherein said composition comprises one or more adeno-associated virus (AAV) vectors comprising a DNA vector construct comprising an insulin gene enhancer protein (ISL1) sequence and/or a DNA vector construct comprising a LIM-homeobox 3 (LHX3) sequence, wherein said ISL1 sequence and/or said LHX3 sequence is operably linked to expression control elements comprising:
(a) a glial fibrillary acid protein (GFAP) promoter;
(b) an enhancer;
(c) a chimeric intron;
(d) a woodchuck hepatitis virus posttranscriptional regulatory element (WPRE); and
(e) and a polyadenylation signal sequence.

92. (canceled)

93. (canceled)

94. The method of claim 91, wherein
(a) the glial fibrillary acidic protein (GFAP) promoter comprises a nucleic acid sequence at least 80% identical to a sequence selected from the group consisting of SEQ ID NOs: 3, 4, and 12;
(b) the enhancer comprises an enhancer from the human elongation factor-1 alpha (EF-1 alpha) promoter comprising a nucleic acid sequence at least 80% identical to SEQ ID NO: 2, or a cytomegalovirus (CMV) enhancer comprising a nucleic acid sequence at least 80% identical to SEQ ID NO: 11;
(c) the chimeric intron comprises a nucleic acid sequence at least 80% identical to SEQ ID NO: 5 or 22;
(d) the woodchuck hepatitis virus posttranscriptional regulatory element (WPRE) comprises a nucleic acid sequence at least 80% identical to SEQ ID NO: 7 or 23; and
(e) the polyadenylation signal sequence comprises a nucleic acid sequence at least 80% identical to a sequence selected from the group consisting of SEQ ID NOs: 8, 17, 24, and 25.

95.-109. (canceled)

110. The method of claim 91, wherein said hISL1 sequence comprises an amino acid sequence at least 80% identical or similar to SEQ ID NO: 10; and/or wherein said hLHX3 sequence comprises an amino acid sequence at least 80% identical or similar to SEQ ID NO: 14.

111.-147. (canceled)

148. The method of claim 91, wherein said at least one glial cell is an astrocyte, a reactive astrocyte, or an NG2 cell.

149.-151. (canceled)

152. The method of claim 91, wherein said functional neuron is selected from the group consisting of glutamatergic neurons, GABAergic neurons, dopaminergic neurons, cholinergic neurons, seratonergic neurons, epinephrinergic neurons, motor neurons, and peptidergic neurons.

153-157. (canceled)

158. The method of claim 91, wherein said subject has a neurological condition comprising an injury to the central nervous system (CNS) or peripheral nervous system.

159. The method of claim 91, wherein said subject has a neurological condition selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.

160.-180. (canceled)


SEQUENCE LISTING (25 sequences)

SEQ ID NO: 1 - DNA, 128 bp, Artificial Sequence
  Description of Artificial Sequence Synthetic polynucleotide
tgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 2 - DNA, 153 bp, Artificial Sequence
  Description of Artificial Sequence Synthetic polynucleotide
tgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggca

SEQ ID NO: 3 - DNA, 686 bp, Artificial Sequence
  Description of Artificial Sequence Synthetic polynucleotide
ctagtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 4 - DNA, 1667 bp, Artificial Sequence
  Description of Artificial Sequence Synthetic polynucleotide
ctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagag

SEQ ID NO: 5 - DNA, 133 bp, Artificial Sequence
  Description of Artificial Sequence Synthetic polynucleotide
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 6 - DNA, 1050 bp, Homo sapiens
atgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccggccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatga

SEQ ID NO: 7 - DNA, 589 bp, Artificial Sequence
  Description of Artificial Sequence Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 8 - DNA, 251 bp, Artificial Sequence
  Description of Artificial Sequence Synthetic polynucleotide
cgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 9 - DNA, 139 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgca

SEQ ID NO: 10 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 11 - DNA, 380 bp, Artificial Sequence
  Description of Artificial Sequence Synthetic polynucleotide
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 12 - DNA, 2214 bp, Homo sapiens
cgcgtcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccggg

SEQ ID NO: 13 - DNA, 1209 bp, Homo sapiens
atggaggcgcgcggggagctgggcccggcccgggagtcggcgggaggcgacctgctgctagcactgctggcgcggagggcggacctgcgccgagagatcccgctgtgcgctggctgtgaccagcacatcctggaccgcttcatcctcaaggctctggaccgccactggcacagcaagtgtctcaagtgcagcgactgccacacgccactggccgagcgctgcttcagccgaggggagagcgtttactgcaaggacgactttttcaagcgcttcgggaccaagtgcgccgcgtgccagctgggcatcccgcccacgcaggtggtgcgccgcgcccaggacttcgtgtaccacctgcactgctttgcctgcgtcgtgtgcaagcggcagctggccacgggcgacgagttctacctcatggaggacagccggctcgtgtgcaaggcggactacgaaaccgccaagcagcgagaggccgaggccacggccaagcggccgcgcacgaccatcaccgccaagcagctggagacgctgaagagcgcttacaacacctcgcccaagccggcgcgccacgtgcgcgagcagctctcgtccgagacgggcctggacatgcgcgtggtgcaggtttggttccagaaccgccgggccaaggagaagaggctgaagaaggacgccggccggcagcgctgggggcagtatttccgcaacatgaagcgctcccgcggcggctccaagtcggacaaggacagcgttcaggaggggcaggacagcgacgctgaggtctccttccccgatgagccttccttggcggaaatgggcccggccaatggcctctacgggagcttgggggaacccacccaggccttgggccggccctcgggagccctgggcaacttctccctggagcatggaggcctggcaggcccagagcagtaccgagagctgcgtcccggcagcccctacggtgtccccccatcccccgccgccccgcagagcctccctggcccccagcccctcctctccagcctggtgtacccagacaccagcttgggccttgtgccctcgggagcccccggcgggcccccacccatgagggtgctggcagggaacggacccagttctgacctatccacggggagcagcgggggttaccccgacttccctgccagccccgcctcctggctggatgaggtagaccacgctcagttctga

SEQ ID NO: 14 - protein, 402 aa, Homo sapiens
MEARGELGPARESAGGDLLLALLARRADLRREIPLCAGCDQHILDRFILKALDRHWHSKCLKCSDCHTPLAERCFSRGESVYCKDDFFKRFGTKCAACQLGIPPTQVVRRAQDFVYHLHCFACVVCKRQLATGDEFYLMEDSRLVCKADYETAKQREAEATAKRPRTTITAKQLETLKSAYNTSPKPARHVREQLSSETGLDMRVVQVWFQNRRAKEKRLKKDAGRQRWGQYFRNMKRSRGGSKSDKDSVQEGQDSDAEVSFPDEPSLAEMGPANGLYGSLGEPTQALGRPSGALGNFSLEHGGLAGPEQYRELRPGSPYGVPPSPAAPQSLPGPQPLLSSLVYPDTSLGLVPSGAPGGPPPMRVLAGNGPSSDLSTGSSGGYPDFPASPASWLDEVDHAQF

SEQ ID NO: 15 - DNA, 57 bp, Artificial Sequence
  Description of Artificial Sequence Synthetic oligonucleotide
gctactaacttcagcctgctgaagcaggctggagacgtggaggagaaccctggacct

SEQ ID NO: 16 - DNA, 54 bp, Artificial Sequence
  Description of Artificial Sequence Synthetic oligonucleotide
gaggggagaggaagtcttctgacctgcggagacgtcgaagagaatcctggaccc

SEQ ID NO: 17 - DNA, 477 bp, Homo sapiens
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 18 - DNA, 66 bp, Artificial Sequence
  Description of Artificial Sequence Synthetic oligonucleotide
ggaagcggagctactaacttcagcctgctgaagcaggctggagacgtggaggagaaccctggacct

SEQ ID NO: 19 - DNA, 66 bp, Artificial Sequence; identical to SEQ ID NO: 18
  Description of Artificial Sequence Synthetic oligonucleotide

SEQ ID NO: 20 - protein, 22 aa, Artificial Sequence
  Description of Artificial Sequence Synthetic peptide
GSGATNFSLLKQAGDVEENPGP

SEQ ID NO: 21 - protein, 21 aa, Artificial Sequence
  Description of Artificial Sequence Synthetic peptide
GSGEGRGSLLTCGDVEENPGP

SEQ ID NO: 22 - DNA, 1062 bp, Artificial Sequence
  Description of Artificial Sequence Synthetic polynucleotide
ggagtcgctgcgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaacgtgctggttgttgtgctgtctcatcattttggcaaagat

SEQ ID NO: 23 - DNA, 581 bp, Artificial Sequence
  Description of Artificial Sequence Synthetic polynucleotide
gagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 24 - DNA, 208 bp, Artificial Sequence
  Description of Artificial Sequence Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 25 - DNA, 49 bp, Artificial Sequence
  Description of Artificial Sequence Synthetic oligonucleotide
cacacaaaaaaccaacacacatccatcttcgatggatagcgattttatt